(19) United States
(12) Patent Application Publication
(10) Pub. No.: US 2025/0000858 A1
(43) Pub. Date: Jan. 2, 2025

(54) FGFR TYROSINE KINASE INHIBITORS FOR THE TREATMENT OF ADVANCED SOLID TUMORS

(71) Applicant: JANSSEN PHARMACEUTICA NV, Beerse (BE)

(72) Inventors: Christopher H. MOY, Raritan, NJ (US); Ademi Elena SANTIAGO-WALKER, Raritan, NJ (US); Michael Edward SCHAFFER, Raritan, NJ (US); Hussein SWEITI, Raritan, NJ (US)

(21) Appl. No.: 18/561,347

(22) PCT Filed: May 19, 2022
(86) PCT No.: PCT/EP2022/063629

§ 371 Date: Nov. 16, 2023

Related U.S. Application Data
(60) Provisional application No. 63/190,602, filed on May 19, 2021.
(60) Provisional application No. 63/242,857, filed on Sep. 10, 2021.
(60) Provisional application No. 63/253,316, filed on Oct. 7, 2021.

(51) Int. Cl. A61K 31/498; A61P 35/00; C12Q 1/6886

(52) U.S. Cl. CPC A61K 31/498; A61P 35/00; C12Q 1/6886; C12Q 2600/156

(57) ABSTRACT

Disclosed herein are methods of treating cancer, said methods comprising administering a therapeutically effective amount of erdafitinib to a patient who has been diagnosed with cancer and who harbors at least one fibroblast growth factor receptor (FGFR) fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. Also disclosed herein are methods of treating cancer comprising: evaluating a biological sample from a patient who has been diagnosed with cancer and who harbors at least one FGFR gene alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, cancer of unknown primary, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; and administering a therapeutically effective dose of an FGFR inhibitor to the patient if at least one FGFR gene alteration is present in the sample.

### TECHNICAL FIELD

Disclosed herein are methods of treating cancer, said methods comprising administering a therapeutically effective amount of erdafitinib to a patient who has been diagnosed with cancer and who harbors at least one fibroblast growth factor receptor (FGFR) fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. Also disclosed herein are methods of treating cancer, said method comprising administering a therapeutically effective amount of erdafitinib to a patient who has been diagnosed with cancer and who harbors at least one FGFR genetic alteration, wherein the cancer is an advanced solid tumor, optionally wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

### BACKGROUND

The identification of genetic abnormalities can be useful in selecting the appropriate therapeutics for cancer patients. Identification of genetic abnormalities is also useful for cancer patients failing the main therapeutic option (front-line therapy) for that cancer type, particularly if there is no accepted standard of care for second and subsequent-line therapy. Fibroblast growth factor receptors (FGFRs) are a family of receptor tyrosine kinases involved in regulating cell survival, proliferation, migration, and differentiation. FGFR alterations may function as oncogenic drivers of disease independent of the underlying tumor type. Little is known about the incidence, diversity or predominant FGFR alterations across solid tumors in the clinical setting.

### SUMMARY

Described herein are methods of treating cancer, said methods comprising, consisting of, or consisting essentially of, administering a therapeutically effective amount of erdafitinib to a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2.

In certain embodiments, the FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. In certain embodiments, the FGFR fusion is selected from FGFR2-CCDC102A, FGFR2-ENOX1, and FGFR2-GPHN.

In certain embodiments, the FGFR fusion is FGFR2-CCDC102A. In further embodiments, the cancer is non-squamous NSCLC.

In certain embodiments, the FGFR fusion is FGFR2-CCDC147. In further embodiments, the FGFR fusion is FGFR2-ENOX1. In still further embodiments, the FGFR fusion is FGFR2-LCN10. In certain embodiments, the FGFR fusion is FGFR2-PDE3A. In further embodiments, the FGFR fusion is FGFR2-RANBP2. In still further embodiments, the FGFR fusion is RRM2B-FGFR2. In certain embodiments, the cancer is cholangiocarcinoma.

In certain embodiments, the FGFR fusion is FGFR2-GPHN. In further embodiments, the cancer is pancreatic cancer.

In certain embodiments, the FGFR fusion is FGFR3-ENOX1. In further embodiments, the FGFR fusion is FGFR3-TMEM247. In certain embodiments, the cancer is a high-grade glioma.

In certain embodiments, the FGFR fusion is IGSF3-FGFR1. In further embodiments, the cancer is a thymic cancer.

In certain embodiments, the FGFR fusion is RHPN2-FGFR1. In further embodiments, the cancer is ovarian cancer.

Described herein are methods of treating cancer, said methods comprising, consisting of, or consisting essentially of, administering a therapeutically effective amount of erdafitinib to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

Also described herein are methods of treating cancer, said methods comprising, consisting of, or consisting essentially of, administering a therapeutically effective amount of erdafitinib to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma.

In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

In certain embodiments, the at least one FGFR genetic alteration is an FGFR mutation or an FGFR fusion, in particular an FGFR mutation or an FGFR fusion with an intact FGFR kinase domain.

In some embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCIL1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In some embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCIL1, FGFR2-AGAP1, FGFR2-AHCYL1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-PTEN, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In some embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCILl, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-ENOX1, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In some embodiments, the at least one FGFR genetic alteration is FGFR2-HTRA1, FGFR2-IMIPA1, FGFR2-CTNND2, FGFR2-YPEL5, FGFR2-SENP6, FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSC1L1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In some embodiments, the at least one FGFR genetic alteration is FGFR1-PLAG1, FGFR2-C382R, BAG4-FGFR1, IGSF3-FGFR1, FGFR1-K656E, FGFR1-MTUS1, RHPN2-FGFR1, FGFR1-TACC1, WHSC1L1-FGFR1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, WHSC1-FGFR3, CD44-FGFR2, FGFR2-CTNND2, FGFR2-FAM24B, FGFR2-GOLGA2, FGFR2-HTRA1, FGFR2-IMIPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, FGFR3-P250R, or FGFR3-R399C.

In some embodiments, the at least one FGFR genetic alteration is FGFR1-PLAG1, FGFR2-C382R, BAG4-FGFR1, IGSF3-FGFR1, FGFR1-K656E, FGFR1-MTUS1, RHPN2-FGFR1, FGFR1-TACC1, WHSC1L1-FGFR1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-ENOX1, FGFR3-MYH14, FGFR3-R248C, FGFR3-S249C, FGFR3-S249F, FGFR3-S371G, FGFR3-TACC3, FGFR3-TMEM247, WHSC1-FGFR3, CD44-FGFR2, FGFR2-CTNND2, FGFR2-FAM24B, FGFR2-GOLGA2, FGFR2-HTRA1, FGFR2-IMIPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, or FGFR3-P250R.

In some embodiments, the at least one FGFR genetic alteration is FGFR1-MTUS1, FGFR1-PLAG1, FGFR1-TACC1, FGFR2-ATAD2, FGFR2-BICC1, FGFR2-CCDC102A, FGFR2-ENOX1, FGFR2-FKBP15, FGFR2-GKAP1, FGFR2-GPHN, FGFR2-KCTD1, FGFR2-KIAA1598, FGFR2-NOL4, FGFR2-PAWR, FGFR2-SENP6, FGFR2-TACC2, FGFR2-TBC1D4, FGFR2-TRA2B, FGFR2-VPS35, FGFR2-WAC, FGFR3-TACC3, FGFR1-K656E, FGFR2-C382R, FGFR2-E565A, FGFR2-F276C, FGFR2-W72C, FGFR2-Y375C, FGFR3-R248C, or FGFR3-S249C.

In certain embodiments, the subject received at least one line of systemic therapy prior to said administration of erdafitinib.

In certain embodiments, the methods or uses described herein further comprise evaluating a biological sample from the patient for the presence of at least one of a FGFR fusion, in particular the at least one fusion as described herein, or at least one FGFR genetic alteration, in particular at least one genetic alteration described herein, prior to said administration of erdafitinib. In certain embodiments, the biological sample is blood, lymph fluid, bone marrow, a solid tumor sample, or any combination thereof.

In further embodiments, erdafitinib is administered daily, in particular once daily. In still further embodiments, erdafitinib is administered orally. In certain embodiments, erdafitinib is administered orally on a continuous daily, in particular once daily, dosing schedule.

In some embodiments, the patient is 15 years of age or older at the date of first administration of the FGFR inhibitor, in particular erdafitinib. In some embodiments, erdafitinib is administered orally at a dose of about 8 mg daily, in particular once daily. In some embodiments, erdafitinib is administered orally at a dose of about 9 mg daily, in particular once daily.

In some embodiments, the patient is between 12 years of age and <15 years of age at the date of first administration of said FGFR inhibitor, in particular erdafitinib. In certain embodiments, erdafitinib is administered at a dose of about 5 mg daily, in particular once daily. In some embodiments, erdafitinib is administered orally at a dose of about 6 mg daily, in particular once daily. In some embodiments, erdafitinib is administered orally at a dose of about 8 mg daily, in particular once daily.

In some embodiments, the patient is between 6 years of age and <12 years of age at the date of first administration of said FGFR inhibitor, in particular erdafitinib. In certain embodiments, erdafitinib is administered at a dose of about 3 mg daily, in particular once daily. In some embodiments, erdafitinib is administered orally at a dose of about 4 mg daily, in particular once daily. In some embodiments, erdafitinib is administered orally at a dose of about 5 mg daily, in particular once daily.

In certain embodiments, erdafitinib is administered in a solid dosage form. In further embodiments, the solid dosage form is a tablet.

Described herein are methods of treating cancer in a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 comprising, consisting of, or consisting essentially of administering a therapeutically effective dose of an FGFR inhibitor to the patient. In an embodiment, the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. In an embodiment, the at least one FGFR fusion is selected from FGFR2-CCDC102A, FGFR2-ENOX1, and FGFR2-GPHN. In an embodiment, the FGFR inhibitor is erdafitinib.

Described herein are methods of treating cancer comprising, consisting of, or consisting essentially of: evaluating a biological sample for the presence of at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 from a patient who has been diagnosed with cancer; and administering a therapeutically effective dose of an FGFR inhibitor to the patient if at least one FGFR fusion is present in the sample. In an embodiment, the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. In an embodiment, the at least one FGFR fusion is selected from FGFR2-CCDC102A, FGFR2-ENOX1, and FGFR2-GPHN. In an embodiment, the FGFR inhibitor is erdafitinib.

Also described herein are methods of treating cancer comprising, consisting of, or consisting essentially of: determining if a patient who has been diagnosed with cancer harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2; and administering a therapeutically effective dose of an FGFR inhibitor to the patient if the patient harbors at least one of the FGFR fusions. In an embodiment, the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. In an embodiment, the at least one FGFR fusion is selected from FGFR2-CCDC102A, FGFR2-ENOX1, and FGFR2-GPHN. In an embodiment, the FGFR inhibitor is erdafitinib.

Also described herein are methods of treating cancer in a patient who has been diagnosed with cancer and who harbors at least one FGFR gene alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; comprising, consisting of, or consisting essentially of administering a therapeutically effective dose of an FGFR inhibitor to the patient if at least one FGFR gene alteration is present in the sample. In an embodiment, the FGFR inhibitor is erdafitinib.

Also described herein are methods of treating cancer in a patient who has been diagnosed with cancer and who harbors at least one FGFR gene alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; comprising, consisting of, or consisting essentially of administering a therapeutically effective dose of an FGFR inhibitor to the patient if at least one FGFR gene alteration is present in the sample. In an embodiment, the FGFR inhibitor is erdafitinib.

In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of treating cancer comprising, consisting of, or consisting essentially of: evaluating a biological sample for the presence of at least one FGFR gene alteration from a patient who has been diagnosed with cancer, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; and administering a therapeutically effective dose of an FGFR inhibitor to the patient if at least one FGFR gene alteration is present in the sample. In an embodiment, the FGFR inhibitor is erdafitinib.

Also described herein are methods of treating cancer comprising, consisting of, or consisting essentially of: evaluating a biological sample for the presence of at least one FGFR gene alteration from a patient who has been diagnosed with cancer, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR gene alteration is present in the sample. In an embodiment, the FGFR inhibitor is erdafitinib.

In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; and administering a therapeutically effective dose of an FGFR inhibitor to the patient if at least one FGFR gene alteration is present in the sample. In an embodiment, the FGFR inhibitor is erdafitinib.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR gene alteration is present in the sample. In an embodiment, the FGFR inhibitor is erdafitinib.

In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Described herein is an FGFR inhibitor for use in the treatment of cancer in a patient who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. The FGFR inhibitor is to be administered at a therapeutically effective dose. In an embodiment, the FGFR inhibitor is erdafitinib.

Described herein is an FGFR inhibitor for use in the treatment of cancer in a patient who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. The FGFR inhibitor is to be administered at a therapeutically effective dose. In an embodiment, the FGFR inhibitor is erdafitinib. In certain embodiments, the FGFR fusion is selected from FGFR2-CCDC102A, FGFR2-ENOX1, and FGFR2-GPHN.

Described herein is an FGFR inhibitor for use in the treatment of cancer in a patient who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2, and wherein the FGFR inhibitor is administered or is to be administered after evaluation of a biological sample from the patient for the presence of at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 and if at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 is present in the sample. The FGFR inhibitor is to be administered at a therapeutically effective dose. In an embodiment, the FGFR inhibitor is erdafitinib.

Described herein is an FGFR inhibitor for use in the treatment of cancer in a patient who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1, and wherein the FGFR inhibitor is administered or is to be administered after evaluation of a biological sample from the patient for the presence of at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1 and if at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1 is present in the sample. The FGFR inhibitor is to be administered at a therapeutically effective dose. In an embodiment, the FGFR inhibitor is erdafitinib. In certain embodiments, the FGFR fusion is selected from FGFR2-CCDC102A, FGFR2-ENOX1, and FGFR2-GPHN.

Also described herein is an FGFR inhibitor for use in the treatment of cancer in a patient who harbors at least one FGFR genetic alteration, and wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma. The FGFR inhibitor is to be administered at a therapeutically effective dose. In an embodiment, the FGFR inhibitor is erdafitinib.

Also described herein is an FGFR inhibitor for use in the treatment of cancer in a patient who harbors at least one FGFR genetic alteration, and wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. The FGFR inhibitor is to be administered at a therapeutically effective dose. In an embodiment, the FGFR inhibitor is erdafitinib. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein is an FGFR inhibitor for use in the treatment of cancer in a patient who harbors at least one FGFR genetic alteration, and wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma, and wherein the FGFR inhibitor is administered or is to be administered after evaluation of a biological sample from the patient for the presence of at least one FGFR genetic alteration and if at least one FGFR genetic alteration is present in the sample. The FGFR inhibitor is to be administered at a therapeutically effective dose. In an embodiment, the FGFR inhibitor is erdafitinib.

Also described herein is an FGFR inhibitor for use in the treatment of cancer in a patient who harbors at least one FGFR genetic alteration, and wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma, and wherein the FGFR inhibitor is administered or is to be administered after evaluation of a biological sample from the patient for the presence of at least one FGFR genetic alteration and if at least one FGFR genetic alteration is present in the sample. The FGFR inhibitor is to be administered at a therapeutically effective dose. In an embodiment, the FGFR inhibitor is erdafitinib. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Further described herein are uses of an FGFR inhibitor for the manufacture of a medicament for the treatment of a patient who has been diagnosed with a cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. The FGFR inhibitor is to be administered at a therapeutically effective dose. In an embodiment, the FGFR inhibitor is erdafitinib.

Further described herein are uses of an FGFR inhibitor for the manufacture of a medicament for the treatment of a patient who has been diagnosed with a cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. The FGFR inhibitor is to be administered at a therapeutically effective dose. In an embodiment, the FGFR inhibitor is erdafitinib. In certain embodiments, the FGFR fusion is selected from FGFR2-CCDC102A, FGFR2-ENOX1, and FGFR2-GPHN.

Further described herein are uses of an FGFR inhibitor for the manufacture of a medicament for the treatment of a patient who has been diagnosed with a cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2, and wherein the FGFR inhibitor is administered or is to be administered after evaluation of a biological sample from the patient for the presence of at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 and if at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RIPN2-FGFR1, and RRM2B-FGFR2 is present in the sample. The FGFR inhibitor is to be administered at a therapeutically effective dose. In an embodiment, the FGFR inhibitor is erdafitinib.

Further described herein are uses of an FGFR inhibitor for the manufacture of a medicament for the treatment of a patient who has been diagnosed with a cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1, and wherein the FGFR inhibitor is administered or is to be administered after evaluation of a biological sample from the patient for the presence of at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1 and if at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1 is present in the sample. The FGFR inhibitor is to be administered at a therapeutically effective dose. In an embodiment, the FGFR inhibitor is erdafitinib. In certain embodiments, the FGFR fusion is selected from FGFR2-CCDC102A, FGFR2-ENOX1, and FGFR2-GPHN.

Still further described herein are uses of an FGFR inhibitor for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma. The FGFR inhibitor is to be administered at a therapeutically effective dose. In an embodiment, the FGFR inhibitor is erdafitinib.

Still further described herein are uses of an FGFR inhibitor for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. The FGFR inhibitor is to be administered at a therapeutically effective dose. In an embodiment, the FGFR inhibitor is erdafitinib. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Still further described herein are uses of an FGFR inhibitor for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma, and wherein the FGFR inhibitor is administered or is to be administered after evaluation of a biological sample from the patient for the presence of at least one FGFR genetic alteration and if at least one FGFR genetic alteration is present in the sample. The FGFR inhibitor is to be administered at a therapeutically effective dose. In an embodiment, the FGFR inhibitor is erdafitinib.

Still further described herein are uses of an FGFR inhibitor for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma, and wherein the FGFR inhibitor is administered or is to be administered after evaluation of a biological sample from the patient for the presence of at least one FGFR genetic alteration and if at least one FGFR genetic alteration is present in the sample. The FGFR inhibitor is to be administered at a therapeutically effective dose. In an embodiment, the FGFR inhibitor is erdafitinib. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

### BRIEF DESCRIPTION OF THE DRAWINGS

The summary, as well as the following detailed description, is further understood when read in conjunction with the appended drawings. For the purpose of illustrating the disclosed methods or uses, the drawings show exemplary embodiments of the methods or uses; however, the methods or uses are not limited to the specific embodiments disclosed. In the drawings:

FIG. 1 is a schematic overview of the clinical study exemplified herein. ^{a}A cap of up to 30 subjects in each tumor histology will be enrolled in the Broad Panel Cohort. ^{b}Enrollment in the Exploratory Cohort is limited to patients with FGFR mutations that do not meet the Broad Panel Cohort molecular eligibility criteria. ^{c}A separate Cholangiocarcinoma Expansion Cohort will enroll subjects with target FGFR mutations or any FGFR gene fusion once the broad Panel Cohort has reached the cap of approximately 30 subjects for cholangiocarcinoma. ^{d} The Pediatric Cohort will enroll 20 children and adolescent subjects who have progressed following prior therapies and who have no acceptable standard therapies, and approximately 6 additional children and adolescent subjects who have newly diagnosed solid tumor and who have no acceptable standard therapies. Adolescent subjects enrolled in the Broad Panel Cohort (≥12 to <18 years) will be analyzed as part of the Broad Panel Cohort and the Pediatric Cohort; therefore 240 subjects in the Broad Panel Cohort can include subjects from Pediatric Cohort.^{e} Subjects with FGFR mutations (exclusive of valine gatekeeper and resistance alterations), and FGFR gene fusion, or FGFR internal tandem duplication are eligible for enrollment in the Pediatric Cohort. ^{f} The list of target FGFR mutations is provided separately in Example 1A and 1B.

FIG. 2 demonstrates instructions for up-titration of erdafitinib based on serum phosphate levels.

FIG. 3A is a pie chart showing the primary tumor diagnosis for the molecular eligible population (N=191) according to the clinical study described in Example 2.

FIG. 3B is a pie chart showing the primary tumor diagnosis for the enrolled population (N=110) according to the clinical study described in Example 2.

FIG. 4 is a waterfall plot of maximal percentage reduction of target lesion from efficacy analysis set.

FIG. 5 is a swim lane plot for treatment duration and response; responders with confirmed CR/PR by investigator.

FIG. 6 is a waterfall plot of maximal percentage reduction of target lesion from baseline—Independent Radiographic Review (Broad Panel Cohort), treated subjects. CCA=Cholangiocarcinoma; HGG=High-grade Glioma; BRST=Breast Cancer; PANCR=Pancreatic Cancer; sqNSCLC=Squamous NSCLC; nonsqNSCLC=Non-squamous NSCLC; CRC=Colorectal Cancer; EDMTL=Endometrial Cancer; ESOPH=Esophageal Cancer; LGG=Low-grade Glioma; GSTRC=Gastric Cancer; HNSCC═Squamous Cell Head and Neck Cancers; CRVX=Cervical Cancer; OVAR=Ovarian Cancer; CR: Complete Response; PR: Partial Response; SD: Stable Disease; PD: Progressive Disease; NE: Inevaluable. The best overall response is the best response recorded from the start of the study treatment to the end of study, prior to PD and subsequent anticancer therapy (subsequent surgery/procedure, subsequent radiotherapy and subsequent systemic therapy), taking into account any requirement for confirmation. For disease evaluations based on RECIST 1.1, maximum percentage reduction from baseline is calculated in sum of target Lesion diameters; while for disease evaluations based on RANO, maximum percentage reduction from baseline is calculated in sum of product of perpendicular dimension. Maximal percentage increase of target lesion from baseline greater than 100% is set to 100%. At the time of the data cut, 1 subject had “Unknown” FGFR mutation/fusion. The subject FGFR status has since been confirmed as FGFR fusion.

FIG. 7 is a swim lane plot for treatment duration and response—Independent Radiographic Review (Broad Panel Cohort); responders with confirmed CR/PR by IRC. CCA=Cholangiocarcinoma; HGG=High-grade Glioma; BRST=Breast Cancer; PANCR=Pancreatic Cancer; sqNSCLC=Squamous NSCLC; nonsqNSCLC=Non-squamous NSCLC; EDMTL=Endometrial Cancer; ESOPH=Esophageal Cancer; LGG=Low-grade Glioma; HNSCC═Squamous Cell Head and Neck Cancers; OVAR=Ovarian Cancer. * indicates that a patient is still on-treatment. + indicates that the duration of response for a patient is currently censored.

FIG. 8 is a forest plot of objective response rate by subgroups—Independent Radiographic Review (Broad Panel Cohort) treated subjects. CR: Complete Response; PR: Partial Response.

### DETAILED DESCRIPTION OF ILLUSTRATIVE EMBODIMENTS

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

### Certain Terminology

The transitional terms “comprising”, “consisting essentially of”, and “consisting” are intended to connote their generally accepted meanings in the patent vernacular; that is, (i) “comprising”, which is synonymous with “including”, “containing”, or “characterized by”, is inclusive or open-ended and does not exclude additional, unrecited elements or method steps; (ii) “consisting of” excludes any element, step, or ingredient not specified in the claim; and (iii) “consisting essentially of” limits the scope of a claim or embodiment to the specified materials or steps “and those that do not materially affect the basic and novel characteristic(s)” of the claimed invention or the embodiment. More specifically, the basic and novel characteristics relates to the ability of the method or use to provide at least one of the benefits described herein, including but not limited to the ability to improve the survivability of the human population relative to the survivability of the comparative human population described elsewhere herein. Embodiments described in terms of the phrase “comprising” (or its equivalents), also provide, as embodiments, those which are independently described in terms of “consisting of” and “consisting essentially of”.

When a value is expressed as an approximation by use of the descriptor “about”, it will be understood that the particular value forms another embodiment. If not otherwise specified, the term “about” signifies a variance of 10% of the associated value, but additional embodiments include those where the variance may be ±5%, ±15%, ±20%, ±25%, or ±50%, in particular the term “about” signifies a variance of ±5% or ±10% of the associated value, more in particular ±5%.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the singular forms “a,” “an,” and “the” include the plural.

As used herein, “patient” is intended to mean any animal, in particular, mammals. Thus, the methods or uses are applicable to human and nonhuman animals, although most preferably with humans. The terms “patient” and “subject” and “human” may be used interchangeably.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “dosage” refers to the information of the amount of the therapeutic to be taken by the subject and the frequency of the number of times the therapeutic is to be taken by the subject.

The term “dose” refers to the amount or quantity of the therapeutic to be taken each time.

The term “cancer” as used herein refers to an abnormal growth of cells which tend to proliferate in an uncontrolled way and, in some cases, to metastasize (spread).

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The terms “co-administration” or the like, as used herein, encompass administration of the selected therapeutic agents to a single patient, and are intended to include treatment regimens in which the agents are administered by the same or different route of administration or at the same or different time.

The term “adverse event” is any untoward medical occurrence in a clinical study subject administered a medicinal (investigational or non-investigational) product. An adverse event does not necessarily have a causal relationship with the intervention. An adverse event can therefore be any unfavorable and unintended sign (including an abnormal finding), symptom, or disease temporally associated with the use of a medicinal (investigational or non-investigational) product, whether or not related to that medicinal (investigational or non-investigational) product.

The term “placebo” as used herein means administration of a pharmaceutical composition that does not include an FGFR inhibitor.

The term “randomization” as it refers to a clinical trial refers to the time when the patient is confirmed eligible for the clinical trial and gets assigned to a treatment arm.

The terms “kit” and “article of manufacture” are used as synonyms.

The terms “objective response rate” and “overall response rate” are used herein interchangeably.

“Biological samples” refers to any sample from a patient in which cancerous cells can be obtained and detection of a FGFR genetic alteration is possible. Suitable biological samples include, but are not limited to, blood, lymph fluid, bone marrow, a solid tumor sample, or any combination thereof. In some embodiments, the biological sample can be formalin-fixed paraffin-embedded tissue (FFPET).

In the context of determining if a patient harbors at least one FGFR genetic alteration, the term “determining” includes a healthcare professional reviewing the result(s) of an evaluation of a biological sample for the presence of one or more FGFR genetic alterations. For example, based upon a review of such results (e.g., a patient's sequencing results by next-generation sequencing, direct sequencing, etc.) a healthcare professional may determine (recognize) that the patient harbors at least one FGFR genetic alteration, such as fusion(s) as described herein. Based on that determination, according to particular embodiments, administration of erdafitinib becomes a part of the patient's treatment regimen.

The term “intact FGFR kinase domain” refers to (a) an FGFR fusion with a 3-prime partner (FGFR gene is listed first, e.g. FGFR-GENE or FGFR3-TACC3) where the FGFR portion of fusion must involve exon ≥17; (b) an FGFR fusion with a 5-prime partner (partner gene is listed first and FGFR gene is second, e.g. GENE-FGFR) where the FGFR portion of the fusion must involve exon <11, or (c) a named FGFR fusion partner gene (self-fusions or rearrangements, e.g. FGFR-FGFR, are not eligible).

### FGFR Genetic Alterations

Described herein are methods of treating cancer, said methods comprising, consisting of, or consisting essentially of, administering a therapeutically effective amount of an FGFR inhibitor, in particular erdafitinib, to a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2.

Described herein are methods of treating cancer, said methods comprising, consisting of, or consisting essentially of, administering a therapeutically effective amount of an FGFR inhibitor, in particular erdafitinib, to a patient who has been diagnosed with cancer, and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1.

Described herein are methods of treating cancer, said methods comprising, consisting of, or consisting essentially of, administering a therapeutically effective amount of an FGFR inhibitor, in particular erdafitinib, to a patient who has been diagnosed with cancer, and who harbors at least one FGFR genetic alteration selected from FGFR1-PLAG1, FGFR2-C382R, BAG4-FGFR1, IGSF3-FGFR1, FGFR1-K656E, FGFR1-MTUS1, RHPN2-FGFR1, FGFR1-TACC1, WHSCILl-FGFR1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, WHSC1-FGFR3, CD44-FGFR2, FGFR2-CTNND2, FGFR2-FAM24B, FGFR2-GOLGA2, FGFR2-HTRA1, FGFR2-IMIPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, FGFR3-P250R, or FGFR3-R399C.

Described herein are methods of treating cancer, said methods comprising, consisting of, or consisting essentially of, administering a therapeutically effective amount of an FGFR inhibitor, in particular erdafitinib, to a patient who has been diagnosed with cancer, and who harbors at least one FGFR genetic alteration selected from FGFR1-MTUS1, FGFR1-PLAG1, FGFR1-TACC1, FGFR2-ATAD2, FGFR2-BICC1, FGFR2-CCDC102A, FGFR2-ENOX1, FGFR2-FKBP15, FGFR2-GKAP1, FGFR2-GPHN, FGFR2-KCTD1, FGFR2-KIAA1598, FGFR2-NOL4, FGFR2-PAWR, FGFR2-SENP6, FGFR2-TACC2, FGFR2-TBC1D4, FGFR2-TRA2B, FGFR2-VPS35, FGFR2-WAC, FGFR3-TACC3, FGFR1-K656E, FGFR2-C382R, FGFR2-E565A, FGFR2-F276C, FGFR2-W72C, FGFR2-Y375C, FGFR3-R248C, or FGFR3-S249C.

In certain embodiments, the patient does not harbor an FGFR valine gatekeeper or resistance alteration, in particular valine gatekeeper or resistance alterations selected from: FGFR1 V561, FGFR2 V564, FGFR3 V555, FGFR4 V550, FGFR1 N546, FGFR2 N549, FGFR3 N540 and FGFR4 N535.

Also described herein are methods of treating cancer, said methods comprising, consisting of, or consisting essentially of, administering a therapeutically effective amount of an FGFR inhibitor, in particular erdafitinib, to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

Also described herein are methods of treating cancer, said methods comprising, consisting of, or consisting essentially of, administering a therapeutically effective amount of an FGFR inhibitor, in particular erdafitinib, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

In certain embodiments, the at least one FGFR genetic alteration is an FGFR mutation or an FGFR fusion, in particular an FGFR mutation or an FGFR fusion with an intact FGFR kinase domain. In certain embodiments, the FGFR fusion is a FGFR1 fusion, in particular a FGFR1 fusion as described herein. In certain embodiments, the FGFR fusion is a FGFR2 fusion, in particular a FGFR2 fusion as described herein. In certain embodiments, the FGFR fusion is a FGFR3 fusion, in particular a FGFR3 fusion as described herein. In certain embodiments, the FGFR fusion is a FGFR1 fusion, a FGFR2 fusion or a FGFR3 fusion, in particular a FGFR1 fusion, a FGFR2 fusion or FGFR3 fusion as described herein. In certain embodiments, the FGFR fusion is a FGFR2 fusion or a FGFR3 fusion, in particular a FGFR2 fusion or FGFR3 fusion as described herein. In certain embodiments, the FGFR mutation is a FGFR2 mutation, in particular a FGFR2 mutation as described herein. In certain embodiments, the FGFR mutation is a FGFR3 mutation, in particular a FGFR3 mutation as described herein. In certain embodiments, the FGFR mutation is a FGFR2 mutation or a FGFR3 mutation, in particular a FGFR2 mutation or a FGFR3 mutation as described herein. In certain embodiments, the indication is an advanced solid tumor with a FGFR1 fusion, in particular a FGFR1 fusion as described herein. In certain embodiments, the indication is an advanced solid tumor with a FGFR2 fusion, in particular a FGFR2 fusion as described herein. In certain embodiments, the indication is an advanced solid tumor with a FGFR3 fusion, in particular a FGFR3 fusion as described herein. In certain embodiments, the indication is an advanced solid tumor with a FGFR1 fusion, a FGFR2 fusion or a FGFR3 fusion, in particular a FGFR1 fusion, a FGFR2 fusion or FGFR3 fusion as described herein. In certain embodiments, the indication is an advanced solid tumor with a FGFR2 fusion or a FGFR3 fusion, in particular a FGFR2 fusion or FGFR3 fusion as described herein. In certain embodiments, the indication is an advanced solid tumor with a FGFR2 mutation, in particular a FGFR2 mutation as described herein. In certain embodiments, the indication is an advanced solid tumor with a FGFR3 mutation, in particular a FGFR3 mutation as described herein. In certain embodiments, the indication is an advanced solid tumor with a FGFR2 mutation or a FGFR3 mutation, in particular a FGFR2 mutation or a FGFR3 mutation as described herein.

Also described herein are methods of treating cancer, said methods comprising, consisting of, or consisting essentially of, administering a therapeutically effective amount of an FGFR inhibitor, in particular erdafitinib, to a pediatric patient who has been diagnosed with cancer and who harbors at least one FGFR genetic alteration, wherein the cancer is glioblastoma multiforme, low grade glioma, pilocytic astrocytoma, rhabdomyosarcoma, Wilms' tumor, neuroblastoma, Ewing sarcoma, or medulloblastoma. Also described herein are methods of treating cancer, said methods comprising, consisting of, or consisting essentially of, administering a therapeutically effective amount of an FGFR inhibitor, in particular erdafitinib, to a pediatric patient who has been diagnosed with cancer and who harbors at least one FGFR genetic alteration, wherein the cancer is dysembryoplastic neuroepithelial tumor, glioblastoma, glioma, rhabdomyosarcoma, Wilms' tumor, neuroblastoma, Ewing sarcoma, or medulloblastoma. In certain embodiments, the glioma comprises low-grade glioma and high-grade glioma. In certain embodiments, low-grade glioma comprises pilocytic astrocytoma, astrocytoma, pilomyxoid astrocytoma, oligoastrocytoma, and pleomorphic xanthoastrocytoma In certain embodiments, high-grade glioma comprises anaplastic astrocytoma. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein are methods of treating glioblastoma multiforme, said methods comprising, consisting of, or consisting essentially of, administering a therapeutically effective amount of an FGFR inhibitor, in particular erdafitinib, to a pediatric patient who has been diagnosed with glioblastoma multiforme and who harbors at least one FGFR genetic alteration. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain. The fibroblast growth factor (FGF) family of protein tyrosine kinase (PTK) receptors regulates a diverse array of physiologic functions including mitogenesis, wound healing, cell differentiation and angiogenesis, and development. Both normal and malignant cell growth as well as proliferation are affected by changes in local concentration of FGFs, extracellular signaling molecules which act as autocrine as well as paracrine factors. Autocrine FGF signaling may be particularly important in the progression of steroid hormone-dependent cancers to a hormone independent state.

FGFs and their receptors are expressed at increased levels in several tissues and cell lines and overexpression is believed to contribute to the malignant phenotype. Furthermore, a number of oncogenes are homologues of genes encoding growth factor receptors, and there is a potential for aberrant activation of FGF-dependent signaling in human pancreatic cancer (Knights et al., Pharmacology and Therapeutics 2010 125:1 (105-117); Korc M. et al Current Cancer Drug Targets 2009 9:5 (639-651)).

The two prototypic members are acidic fibroblast growth factor (aFGF or FGF1) and basic fibroblast growth factor (bFGF or FGF2), and to date, at least twenty distinct FGF family members have been identified. The cellular response to FGFs is transmitted via four types of high affinity transmembrane protein tyrosine-kinase fibroblast growth factor receptors (FGFR) numbered 1 to 4 (FGFR1 to FGFR4).

In certain embodiments, the cancer is susceptible to an FGFR genetic alteration.

As used herein, “FGFR genetic alteration” refers to an alteration in the wild type FGFR gene, including, but not limited to, FGFR fusion genes, FGFR mutations, or any combination thereof. The terms “variant” and “alteration” are used interchangeably herein.

In certain embodiments, the FGFR genetic alteration is an FGFR gene fusion. “FGFR fusion” or “FGFR gene fusion” refers to a gene encoding a portion of FGFR (e.g., FGRF2 or FGFR3) and one of the herein disclosed fusion partners, or a portion thereof, created by a translocation between the two genes. The terms “fusion” and “translocation” are used interchangeable herein. Table 9, Table 14, and Table 19 provide the FGFR fusion genes and the FGFR and fusion partner.

Table 1 provides a list of exemplary FGFR fusions and gene breakpoints.

TABLE 1

| | | GENE1 | GENE2 |
|---|---|---|---|
| Fusion | Histology | Breakpoint | Breakpoint |
| FGFR2- | CHOLANGIOCARCINOMA | chr10: 123243212 | chr13: 43843713 |
| ENOX1 | | (−) | (−) |
| FGFR2- | PANCREATIC CANCER | chr10: 123242877- | chr14: 67490180- |
| GPHN | | 123243168 | 67490353 |
| FGFR2- | CHOLANGIOCARCINOMA | chr10: 123239533 | chr2: 109351998 |
| RANBP2 | | | |
| FGFR3- | HIGH-GRADE GLIOMAS | chr4: 1808661 | chr13: 43896636 |
| ENOX1 | (EG. GLIOBLASTOMA) | | |
| IGSF3- | THYMIC CANCER | chr1: 116613765 | chr8: 38428435 |
| FGFR1 | | (hg38) | (hg38) |
| RHPN2- | OVARIAN CANCER | chr19: 33535154 | chr8: 38315051 |
| FGFR1 | | | |
| RRM2B- | CHOLANGIOCARCINOMA | chr8: 103246953 | chr10: 123298229 |
| FGFR2 | | (exon 2) | (exon 5) |

In any of the described embodiments, the FGFR fusion may by any FGFR fusion wherein the FGFR protein has an intact FGFR kinase domain. In certain embodiments, the at least one FGFR fusion is selected from FGFR1-PLAG1, FGFR1-BAG4, IGSF3-FGFR1, FGFR1-MTUS1, FGFR1-RHPN2, FGFR1-TACC1, FGFR1-WHSCILl, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-ENOX1, FGFR2-FKBP15, FGFR2-GKAP1, FGFR2-GPHN, FGFR2-KCTD1, FGFR2-KIAA1598, FGFR2-KIF6, FGFR2-LGSN, FGFR2-NOL4, FGFR2-NRBF2, FGFR2-PAWR, FGFR2-PDE3A, FGFR2-POC1B, FGFR2-SYNPO2, FGFR2-TACC2, FGFR2-TBC1D4, FGFR2-TBC1D5, FGFR2-TCERG1L, FGFR2-TRA2B, FGFR2-VPS35, FGFR2-WAC, FGFR3-ENOX1, FGFR3-MYH14, FGFR3-TACC3, FGFR3-TMEM247, and FGFR3-WHSC1.

In certain embodiments, the at least one FGFR fusion is selected from FGFR1-BAG4, IGSF3-FGFR1, FGFR1-MTUS1, FGFR1-RHPN2, FGFR1-TACC1, FGFR1-WHSC1L1, FGFR2-AGAP1, FGFR2-AHCYL1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-ENOX1, FGFR2-FKBP15, FGFR2-GKAP1, FGFR2-GPHN, FGFR2-KCTD1, FGFR2-KIAA1598, FGFR2-KIF6, FGFR2-LGSN, FGFR2-NOL4, FGFR2-NRBF2, FGFR2-PAWR, FGFR2-PDE3A, FGFR2-POC1B, FGFR2-PTEN, FGFR2-SYNPO2, FGFR2-TACC2, FGFR2-TBC1D4, FGFR2-TBC1D5, FGFR2-TCERG1L, FGFR2-TRA2B, FGFR2-WAC, FGFR3-ENOX1, FGFR3-MYH14, FGFR3-TACC3, FGFR3-TMEM247, and FGFR3-WHSC1.

In certain embodiments, the at least one FGFR fusion is selected from FGFR2-HTRA1, FGFR2-IMIPA1, FGFR2-CTNND2, FGFR2-YPEL5, FGFR2-SENP6, FGFR1-PLAG1, FGFR1-BAG4, IGSF3-FGFR1, FGFR1-MTUS1, FGFR1-RHPN2, FGFR1-TACC1, FGFR1-WHSCILl, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-ENOX1, FGFR2-FKBP15, FGFR2-GKAP1, FGFR2-GPHN, FGFR2-KCTD1, FGFR2-KIAA1598, FGFR2-KIF6, FGFR2-LGSN, FGFR2-NOL4, FGFR2-NRBF2, FGFR2-PAWR, FGFR2-PDE3A, FGFR2-POC1B, FGFR2-SYNPO2, FGFR2-TACC2, FGFR2-TBC1D4, FGFR2-TBC1D5, FGFR2-TCERG1L, FGFR2-TRA2B, FGFR2-VPS35, FGFR2-WAC, FGFR3-ENOX1, FGFR3-MYH14, FGFR3-TACC3, FGFR3-TMEM247, and FGFR3-WHSC1.

In certain embodiments, the at least one FGFR fusion is selected from BAG4-FGFR1, CD44-FGFR2, FGFR1-MTUS1, FGFR1-PLAG1, FGFR1-TACC1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-CTNND2, FGFR2-ENOX1, FGFR2-FAM24B, FGFR2-FKBP15, FGFR2-GKAP1, FGFR2-GOLGA2, FGFR2-GPHN, FGFR2-HTRA1, FGFR2-IMIPA1, FGFR2-KCTD1, FGFR2-KIAA1598, FGFR2-KIF6, FGFR2-LGSN, FGFR2-NOL4, FGFR2-NRBF2, FGFR2-PAWR, FGFR2-PDE3A, FGFR2-POC1B, FGFR2-SENP6, FGFR2-SYNPO2, FGFR2-TACC2, FGFR2-TBC1D4, FGFR2-TBC1D5, FGFR2-TCERG1L, FGFR2-TRA2B, FGFR2-VPS35, FGFR2-WAC, FGFR2-YPEL5, FGFR3-ENOX1, FGFR3-JAKMIP1, FGFR3-MYH14, FGFR3-TACC3, FGFR3-TMEM247, IGSF3-FGFR1, RHPN2-FGFR1, WDR11-FGFR2, WHSCl-FGFR3, and WHSC1L1-FGFR1.

In certain embodiments, the at least one FGFR fusion is selected from FGFR1-MTUS1, FGFR1-PLAG1, FGFR1-TACC1, FGFR2-ATAD2, FGFR2-BICC1, FGFR2-CCDC102A, FGFR2-ENOX1, FGFR2-FKBP15, FGFR2-GKAP1, FGFR2-GPHN, FGFR2-KCTD1, FGFR2-KIAA1598, FGFR2-NOL4, FGFR2-PAWR, FGFR2-SENP6, FGFR2-TACC2, FGFR2-TBC1D4, FGFR2-TRA2B, FGFR2-VPS35, FGFR2-WAC, and FGFR3-TACC3.

In certain embodiments, the at least one FGFR fusion is selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2.

In certain embodiments, the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1.

FGFR genetic alterations include FGFR single nucleotide polymorphism (SNP). “FGFR single nucleotide polymorphism” (SNP) refers to a FGFR gene in which a single nucleotide differs among individuals. In certain embodiments, the FGFR genetic alteration is an FGFR3 gene mutation. In particular, FGFR single nucleotide polymorphism” (SNP) refers to a FGFR1, FGFR2, or FGFR3 gene in which a single nucleotide differs among individuals. The presence of one or more of the FGFR SNPs in Table 9, Table 14, or Table 19 in a biological sample from a patient can be determined by methods known to those of ordinary skill in the art or methods disclosed in WO 2016/048833.

In certain embodiments, the at least one FGFR mutation is selected from FGFR1-K656E, FGFR2-C382R, FGFR2-D101Y, FGFR2-F276C, FGFR2-K659M, FGFR2-L551F, FGFR2-L770V, FGFR2-S252L, FGFR2-S267P, FGFR2-V395D, FGFR2-Y375C, FGFR3-A500T, FGFR3-F384L, FGFR3-R248C, FGFR3-S249C, FGFR3-S249F, and FGFR3-S371G.

In certain embodiments, the at least one FGFR mutation is selected from FGFR1-K656E, FGFR2-C382R, FGFR2-D101Y, FGFR2-F276C, FGFR2-K659M, FGFR2-L551F, FGFR2-L770V, FGFR2-S252L, FGFR2-S267P, FGFR2-V395D, FGFR2-Y375C, FGFR3-R248C, FGFR3-S249C, FGFR3-S249F, and FGFR3-S371G.

In certain embodiments, the at least one FGFR mutation is selected from FGFR1-K656E, FGFR1-S125L, FGFR2-C382R, FGFR2-D101Y, FGFR2-E565A, FGFR2-F276C, FGFR2-K659M, FGFR2-L551F, FGFR2-L770V, FGFR2-P253L, FGFR2-S252L, FGFR2-S267P, FGFR2-V395D, FGFR2-W72C, FGFR2-Y375C, FGFR3-A500T, FGFR3-F384L, FGFR3-P250R, FGFR3-R248C, FGFR3-R399C, FGFR3-S249C, FGFR3-S249F, and FGFR3-S371G.

In certain embodiments, the at least one FGFR mutation is selected from FGFR1-K656E, FGFR2-C382R, FGFR2-E565A, FGFR2-F276C, FGFR2-W72C, FGFR2-Y375C, FGFR3-R248C, and FGFR3-S249C.

In certain embodiments, the at least one FGFR mutation is not an FGFR valine gatekeeper or resistance alteration. In certain embodiments, the at least one FGFR mutation is not FGFR1 V561, FGFR2 V564, FGFR3 V555, FGFR4 V550, FGFR1 N546,

### FGFR2 N549, FGFR3 N540 or FGFR4 N535.

As used herein, “FGFR genetic alteration gene panel” includes one or more of the above listed FGFR genetic alterations. In some embodiments, the FGFR genetic alteration gene panel is dependent upon the patient's cancer type.

The FGFR genetic alteration gene panel that is used in the evaluating step of the disclosed methods is based, in part, on the patient's cancer type. For patients with cancer, a suitable FGFR genetic alteration gene panel can comprise any of the FGFR genetic alterations disclosed in Table 9, Table 14, or Table 19. In an embodiment, for patients with cancer, a suitable FGFR genetic alteration gene panel can comprise any of the FGFR genetic alterations disclosed in target FGFR mutations of Example 1A. In an embodiment, for patients with cancer, a suitable FGFR genetic alteration gene panel can comprise any of the FGFR genetic alterations disclosed in target FGFR mutations of Example 1B

### FGFR Inhibitors for Use in the Disclosed Methods or Uses

Suitable FGFR inhibitors for use in the disclosed methods or uses are provided herein. The FGFR inhibitors may be used alone or in combination for the treatment methods described herein.

In some embodiments, if one or more FGFR genetic alterations are present in the sample, the cancer can be treated with a FGFR inhibitor disclosed in U.S. Publication No. 2013/0072457 A1 (incorporated herein by reference), including any tautomeric or stereochemically isomeric form thereof, and a N-oxide thereof, a pharmaceutically acceptable salt thereof, or a solvate thereof.

In some aspects, for example, the cancer may be treated with N-(3,5-dimethoxy-phenyl)-N′-(1-methylethyl)-N-[3-(1-methyl-1H-pyrazol-4-yl)quinoxalin-6-yl]ethane-1,2-diamine (referred to herein “JNJ-42756493” or “JNJ493” or erdafitinib), including any tautomeric form thereof, N-oxides thereof, pharmaceutically acceptable salts thereof, or solvates thereof. In some embodiments, the FGFR inhibitor can be the compound of formula (I), also referred to as erdafitinib:

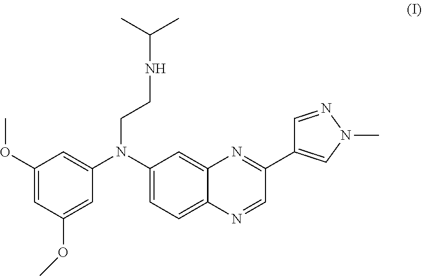 [SMILES: COc1cc(OC)cc(N(CCNC(C)C)c2ccc3ncc(-c4cnn(C)c4)nc3c2)c1]

or a pharmaceutically acceptable salt thereof. In some aspects, the pharmaceutically acceptable salt is a HCl salt. In preferred aspects, erdafitinib base is used.

Erdafitinib (also referred to as ERDA), an oral pan-FGFR kinase inhibitor, has been approved by the U.S. Food and Drug Administration (FDA) for the treatment of adult patients who have locally advanced UC or mUC which has susceptible FGFR3 or FGFR2 genetic alterations and who have progressed during or following at least one line of prior platinum-containing chemotherapy, including within 12 months of neoadjuvant or adjuvant platinum-containing chemotherapy. Loriot Y et al. NEJM. 2019; 381:338-48. Erdafitinib has shown clinical benefits and tolerability in patients with mUC and alteration in FGFR expressions. Tabernero J, et al. J Clin Oncol. 2015; 33:3401-3408; Soria J-C, et al. Ann Oncol. 2016; 27(Suppl 6):vi266-vi295. Abstract 781PD; Siefker-Radtke A O, et al. ASCO 2018. Abstract 4503; Siefker-Radtke A, et al. ASCO-GU 2018. Abstract 450.

In some embodiments, the cancer can be treated with a FGFR inhibitor wherein the FGFR inhibitor is N-[5-[2-(3,5-Dimethoxyphenyl)ethyl]-2H-pyrazol-3-yl]-4-(3,5-diemthylpiperazin-1-yl)benzamide (AZD4547), as described in Gavine, P. R., et al., AZD4547: An Orally Bioavailable, Potent, and Selective Inhibitor of the Fibroblast Growth Factor Receptor Tyrosine Kinase Family, Cancer Res. Apr. 15, 2012 72; 2045:

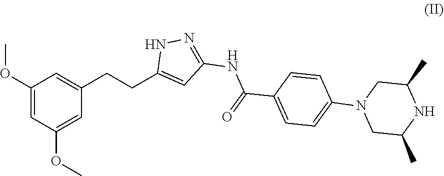 [SMILES: COc1cc(CCc2cc(NC(=O)c3ccc(N4C[C@@H](C)N[C@@H](C)C4)cc3)n[nH]2)cc(OC)c1]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor wherein the FGFR inhibitor is 3-(2,6-Dichloro-3,5-dimethoxy-phenyl)-1-{6-[4-(4 ethyl-piperazin-1-yl)-phenylamino]-pyrimid-4-yl}-methyl-urea (also known as NVP-BGJ398 or infigratinib) as described in Int'l Publ. No. WO2006/000420:

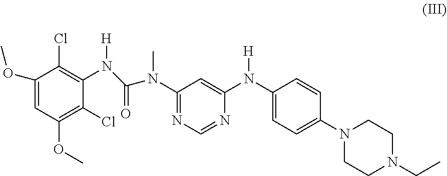 [SMILES: CCN1CCN(c2ccc(Nc3cc(N(C)C(=O)Nc4c(Cl)c(OC)cc(OC)c4Cl)ncn3)cc2)CC1]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor wherein the FGFR inhibitor is 4-amino-5-fluoro-3-[6-(4-methylpiperazin-1-yl)-lH-benzimidazol-2-yl]-1H-quinolin-2-one (dovitinib) as described in Int't Publ. No. WO2006/127926:

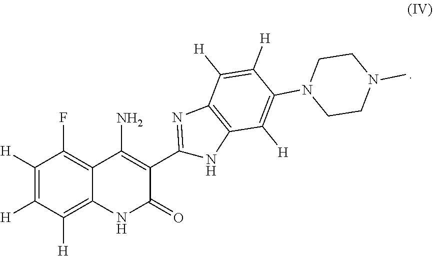 [SMILES: CN1CCN(c2ccc3nc(-c4c(N)c5c(F)cccc5[nH]c4=O)[nH]c3c2)CC1]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor wherein the FGFR inhibitor is 6-(7-((1-Aminocyclopropyl)-methoxy)-6-methoxyquinolin-4-yloxy)-N-methyl-1-naphthamide (AL3810) (lucitanib; E-3810), as described in Bello, E. et al., E-3810 Is a Potent Dual Inhibitor of VEGFR and FGFR that Exerts Antitumor Activity in Multiple Preclinical Models, Cancer Res Feb. 15, 2011 71(A)1396-1405 and Int'l Publ. No. WO2008/112408:

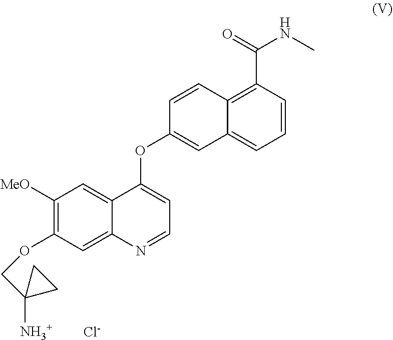 [SMILES: CNC(=O)c1cccc2cc(Oc3ccnc4cc(OCC5([NH3+])CC5)c(OC)cc34)ccc12 ; [Cl-]]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor, wherein the FGFR inhibitor is (4-{[4-amino-6-(methoxymethyl)-5-(7-methoxy-5-methyl-1-benzothiophen-2-yl)pyrrolo[2,1-f][1,2,4]triazin-7-yl]methyl}piperazin-2-one) (also known as BAY1163877 or rogaratinib), as described in Grunewald et al., Rogaratinib: A potent and selective pan-FGFR inhibitor with broad antitumor activity in FGFR-overexpressing preclinical cancer models, Int Journal of Cancer 145(5), 2019:

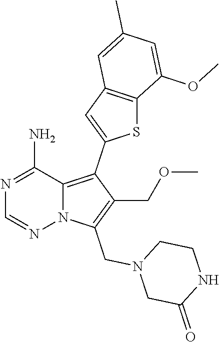 [SMILES: COCc1c(-c2cc3cc(C)cc(OC)c3s2)c2c(N)ncnn2c1CN1CCNC(=O)C1]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor, wherein the FGFR inhibitor is (1-[(3S)-[4-amino-3-[(3,5-dimethoxyphenyl)ethynyl]-1H-pyrazolo[3,4-d]pyrimidin-1-yl]-1-pyrrolidinyl]-2-propen-1-one) (also known as TAS-120 or futibatinib) as described in Sootome et al., Futibatinib Is a Novel Irreversible FGFR 1-4 Inhibitor That Shows Selective Antitumor Activity against FGFR-Deregulated Tumors, Cancer Res; 80(22) Nov. 15, 2020:

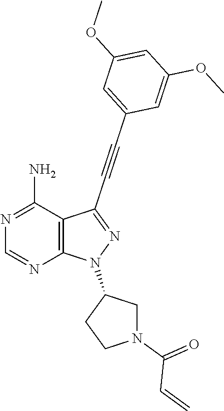 [SMILES: C=CC(=O)N1CC[C@H](n2nc(C#Cc3cc(OC)cc(OC)c3)c3c(N)ncnc32)C1]

including, when chemically possible, any tautomeric or stereochemically isomeric form thereof, and a N-oxide thereof, a pharmaceutically acceptable salt thereof, or a solvate thereof.

In some embodiments, the cancer can be treated with a FGFR inhibitor, wherein the FGFR inhibitor is 3-(2,6-difluoro-3,5-dimethoxyphenyl)1-ethyl-8-(morpholin-4-ylmethyl)-1,3,4,7-tetrahydro-2H-pyrrolo[3′,2′:5,6]pyrido[4,3d]pyrimidin-2-one. (also known as pemigatinib or Pemazyre®):

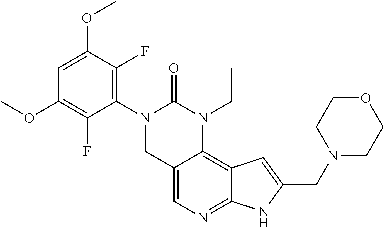 [SMILES: CCN1C(=O)N(c2c(F)c(OC)cc(OC)c2F)Cc2cnc3[nH]c(CN4CCOCC4)cc3c21]

including, when chemically possible, any tautomeric or stereochemically isomeric form thereof, and a N-oxide thereof, a pharmaceutically acceptable salt thereof, or a solvate thereof.

Additional suitable FGFR inhibitors include BAY1179470 (Bayer), ARQ087 (ArQule), ASP5878 (Astellas), FF284 (Chugai), FP-1039 (GSK/FivePrime), Blueprint, LY-2874455 (Lilly), RG-7444 (Roche), or any combination thereof, including, when chemically possible, any tautomeric or stereochemical isomeric forms thereof, N-oxides thereof, pharmaceutically acceptable salts thereof, or solvates thereof.

In an embodiment the FGFR inhibitor generally, and erdafitinib more specifically, is administered as a pharmaceutically acceptable salt. In a preferred embodiment the FGFR inhibitor generally, and erdafitinib more specifically, is administered in base form. In an embodiment the FGFR inhibitor generally, and erdafitinib more specifically, is administered as a pharmaceutically acceptable salt in an amount corresponding to 5 mg base equivalent, 6 mg base equivalent, 8 mg base equivalent, or 9 mg base equivalent. In an embodiment the FGFR inhibitor generally, and erdafitinib more specifically, is administered in base form in an amount of 5 mg, 6 mg, 8 mg or 9 mg. In an embodiment the FGFR inhibitor generally, and erdafitinib more specifically, is administered as a pharmaceutically acceptable salt in an amount corresponding to 3 mg base equivalent or 4 mg base equivalent. In an embodiment the FGFR inhibitor generally, and erdafitinib more specifically, is administered in base form in an amount of 3 mg or 4 mg.

The salts can be prepared by for instance reacting the FGFR inhibitor generally, and erdafitinib more specifically, with an appropriate acid in an appropriate solvent.

Acid addition salts may be formed with acids, both inorganic and organic. Examples of acid addition salts include salts formed with an acid selected from the group consisting of acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids. Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

In an embodiment, the FGFR inhibitor generally, and erdafitinib more specifically, is administered in the form of a solvate. As used herein, the term “solvate” means a physical association of erdafitinib with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. The term “solvate” is intended to encompass both solution-phase and isolatable solvates. Non-limiting examples of solvents that may form solvates include water, isopropanol, ethanol, methanol, DMSO, ethyl acetate, acetic acid or ethanolamine and the like.

Solvates are well known in pharmaceutical chemistry. They can be important to the processes for the preparation of a substance (e.g. in relation to their purification, the storage of the substance (e.g. its stability) and the ease of handling of the substance and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid-State NMR (SS-NMR, also known as Magic Angle Spinning NMR or MAS-NMR). Such techniques are as much a part of the standard analytical toolkit of the skilled chemist as NMR, IR, HPLC and MS. Alternatively the skilled person can deliberately form a solvate using crystallization conditions that include an amount of the solvent required for the particular solvate. Thereafter the standard methods described above, can be used to establish whether solvates had formed. Also encompassed are any complexes (e.g. inclusion complexes or clathrates with compounds such as cyclodextrins, or complexes with metals).

Furthermore, the compound may have one or more polymorph (crystalline) or amorphous forms.

The compounds include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or nonradioactive. In one embodiment, the compounds contain no radioactive isotopes. Such compounds are preferred for therapeutic use. In another embodiment, however, the compound may contain one or more radioisotopes. Compounds containing such radioisotopes may be useful in a diagnostic context.

### Methods of Treatment and Uses

Described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. In certain embodiments, the cancer is NSCLC, in particular non-squamous NSCLC, cholangiocarcinoma, pancreatic cancer, high-grade glioma, thymic cancer, or ovarian cancer.

Described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. In certain embodiments, the cancer is non-squamous NSCLC, cholangiocarcinoma, pancreatic cancer, high-grade glioma, thymic cancer, or ovarian cancer.

Described herein are methods of treating NSCLC, said methods comprising, consisting of, or consisting essentially of, administering a therapeutically effective amount of an FGFR inhibitor generally, and erdafitinib more specifically, to a patient who has been diagnosed with NSCLC, in particular non-squamous NSCLC, and who harbors at least one FGFR fusion. In certain embodiments, the at least one FGFR fusion is FGFR2-CCDC102A.

Described herein are methods of treating cholangiocarcinoma, said methods comprising, consisting of, or consisting essentially of, administering a therapeutically effective amount of an FGFR inhibitor generally, and erdafitinib more specifically, to a patient who has been diagnosed with cholangiocarcinoma and who harbors at least one FGFR fusion. In certain embodiments, the at least one FGFR fusion is FGFR2-CCDC147, FGFR2-ENOX1, FGFR2-LCN10, FGFR2-PDE3A, FGFR2-RANBP2, or RRM2B-FGFR2. In certain embodiments, the at least one FGFR fusion is FGFR2-ENOX1, or FGFR2-PDE3A.

Described herein are methods of treating pancreatic cancer, said methods comprising, consisting of, or consisting essentially of, administering a therapeutically effective amount of an FGFR inhibitor generally, and erdafitinib more specifically, to a patient who has been diagnosed with pancreatic cancer and who harbors at least one FGFR fusion. In certain embodiments, the FGFR fusion is FGFR2-GPHN.

Described herein are methods of treating a high-grade glioma, said methods comprising, consisting of, or consisting essentially of, administering a therapeutically effective amount of an FGFR inhibitor generally, and erdafitinib more specifically, to a patient who has been diagnosed with a high-grade glioma and who harbors at least one FGFR fusion. In certain embodiments, the FGFR fusion is FGFR3-ENOX1.

Described herein are methods of treating thymic cancer, said methods comprising, consisting of, or consisting essentially of, administering a therapeutically effective amount of an FGFR inhibitor generally, and erdafitinib more specifically, to a patient who has been diagnosed with thymic cancer and who harbors at least one FGFR fusion. In certain embodiments, the FGFR fusion is IGSF3-FGFR1.

Described herein are methods of treating ovarian cancer, said methods comprising, consisting of, or consisting essentially of, administering a therapeutically effective amount of an FGFR inhibitor generally, and erdafitinib more specifically, to a patient who has been diagnosed with ovarian cancer and who harbors at least one FGFR fusion. In certain embodiments, the FGFR fusion is RHPN2-FGFR1.

Also described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

Also described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma.

Also described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

In certain embodiments, the at least one FGFR genetic alteration is an FGFR mutation or an FGFR fusion.

Also described herein are methods of treating cancer, said methods comprising, consisting of, or consisting essentially of, administering a therapeutically effective amount of an FGFR inhibitor generally, and erdafitinib more specifically, to a patient who has been diagnosed with an advanced solid tumor and who harbors target FGFR mutations or fusions and who has progressed on or after a minimum of 1 line of systemic therapy and for whom there are no remaining therapeutic options with established clinical benefit. In certain embodiments, the patient was unable to tolerate standard of care therapies for the underlying tumor type. In an embodiment, the target FGFR mutations or fusions are as described in an embodiment herein. In an embodiment the target FGFR mutations or fusions is an FGFR mutation selected from target FGFR mutations of Example 1A or selected from target FGFR mutations of Example 1B. In an embodiment the target FGFR mutations or fusions is an FGFR fusion with an intact FGFR kinase domain. In an embodiment the target FGFR mutations or fusions is an FGFR mutation selected from target FGFR mutations of Example 1A or selected from target FGFR mutations of Example 1B or is an FGFR fusions selected from an FGFR fusion with an intact FGFR kinase domain. In an embodiment, the FGFR inhibitor is erdafitinib.

Also described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma.

Also described herein are methods of treating cancer, said methods comprising, consisting of, or consisting essentially of, administering a therapeutically effective amount of an FGFR inhibitor generally, and erdafitinib more specifically, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

In certain embodiments, the at least one FGFR genetic alteration is an FGFR mutation or an FGFR fusion. In an embodiment, the at least one FGFR genetic alteration, at least one FGFR mutation or at least one FGFR fusion is as described in an embodiment herein. In an embodiment the at least one FGFR genetic alteration is selected from target FGFR mutations of Example 1A or selected from target FGFR mutations of Example 1B. In an embodiment the at least one FGFR genetic alteration is an FGFR fusion with an intact FGFR kinase domain. In an embodiment the at least one FGFR genetic alteration is selected from target FGFR mutations of Example 1A or selected from target FGFR mutations of Example 1B or selected from an FGFR fusion with an intact FGFR kinase domain. In an embodiment, the FGFR inhibitor is erdafitinib.

Also described herein are methods of treating cholangiocarcinoma, said methods comprising, consisting of, or consisting essentially of, administering a therapeutically effective amount of an FGFR inhibitor generally, and erdafitinib more specifically, to a patient who has been diagnosed with cholangiocarcinoma and who harbors at least one FGFR genetic alteration, wherein the at least one FGFR genetic alteration is an FGFR fusion. In certain embodiments, the at least one FGFR fusion is selected from FGFR2-AHCYL1, FGFR2-AMOT, FGFR2-BICC1, FGFR2-CD2AP, FGFR2-CFAP57, FGFR2-ENOX1, FGFR2-KIAA1598, FGFR2-LGSN, FGFR2-NOL4, FGFR2-PAWR, FGFR2-PDE3A, FGFR2-POC1B, FGFR2-SYNPO2, FGFR2-TACC2, FGFR2-TBC1D4, FGFR2-TRA2B, FGFR2-WAC, and FGFR3-TACC3. In certain embodiments, the at least one FGFR fusion is selected from FGFR2-CD2AP, FGFR2-CFAP57, FGFR2-ENOX1, FGFR2-KIAA1598, FGFR2-LGSN, FGFR2-NOL4, FGFR2-PAWR, FGFR2-PDE3A, FGFR2-POC1B, FGFR2-SYNPO2, FGFR2-TACC2, FGFR2-TBC1D4, FGFR2-TRA2B, FGFR2-WAC, and FGFR3-TACC3. In an embodiment, the at least one FGFR fusion is FGFR2-AHCYL1. In an embodiment, the at least one FGFR fusion is FGFR2-AMOT. In an embodiment, the at least one FGFR fusion is FGFR2-BICC1. In an embodiment, the at least one FGFR fusion is FGFR2-CD2AP. In an embodiment, the at least one FGFR fusion is FGFR2-CFAP57. In an embodiment, the at least one FGFR fusion is FGFR2-ENOX1. In an embodiment, the at least one FGFR fusion is FGFR2-KIAA1598. In an embodiment, the at least one FGFR fusion is FGFR2-LGSN. In an embodiment, the at least one FGFR fusion is FGFR2-NOL4. In an embodiment, the at least one FGFR fusion is FGFR2-PAWR. In an embodiment, the at least one FGFR fusion is FGFR2-PDE3A. In an embodiment, the at least one FGFR fusion is FGFR2-POC1B. In an embodiment, the at least one FGFR fusion is FGFR2-SYNPO2. In an embodiment, the at least one FGFR fusion is FGFR2-TACC2. In an embodiment, the at least one FGFR fusion is FGFR2-TBC1D4. In an embodiment, the at least one FGFR fusion is FGFR2-TRA2B. In an embodiment, the at least one FGFR fusion is FGFR2-WAC. In an embodiment, the at least one FGFR fusion is FGFR3-TACC3.

Also described herein are methods of treating cholangiocarcinoma, said methods comprising, consisting of, or consisting essentially of, administering a therapeutically effective amount of an FGFR inhibitor generally, and erdafitinib more specifically, to a patient who has been diagnosed with cholangiocarcinoma and who harbors at least one FGFR genetic alteration, wherein the at least one FGFR genetic alteration is an FGFR mutation, in particular a FGFR2 fusion. In certain embodiments, the at least one FGFR mutation is selected from FGFR2-C382R, and FGFR2-V395D. In an embodiment, the at least one FGFR mutation is FGFR2-C382R. In an embodiment, the at least one FGFR mutation is FGFR2-V395D.

Also described herein are methods of treating high-grade glioma, said methods comprising, consisting of, or consisting essentially of, administering a therapeutically effective amount of an FGFR inhibitor generally, and erdafitinib more specifically, to a patient who has been diagnosed with high-grade glioma and who harbors at least one FGFR genetic alteration, wherein the at least one FGFR genetic alteration is an FGFR fusion, in particular a FGFR1, FGFR2 or a FGFR3 fusion. In certain embodiments, the at least one FGFR fusion is selected from FGFR1-TACC1, FGFR3-ENOX1, FGFR3-MYH14, FGFR3-TACC3, FGFR3-TMEM247 and FGFR2-IMPA1 or is selected from FGFR1-TACC1, FGFR3-ENOX1, FGFR3-MYH14, FGFR3-TACC3, and FGFR3-TMEM247. In an embodiment, the at least one FGFR fusion is FGFR1-TACC1. In an embodiment, the at least one FGFR fusion is FGFR3-ENOX1. In an embodiment, the at least one FGFR fusion is FGFR3-MYH14. In an embodiment, the at least one FGFR fusion is FGFR3-TACC3. In an embodiment, the at least one FGFR fusion is FGFR3-TMEM247. In an embodiment, the FGFR fusion is FGFR2-IMIPA1.

Also described herein are methods of treating high-grade glioma, said methods comprising, consisting of, or consisting essentially of, administering a therapeutically effective amount of an FGFR inhibitor generally, and erdafitinib more specifically, to a patient who has been diagnosed with high-grade glioma and who harbors at least one FGFR genetic alteration, wherein the at least one FGFR genetic alteration is an FGFR mutation. In an embodiment, the FGFR mutation is an FGFR1 mutation, and FGFR2 mutation, or an FGFR3 mutation.

Also described herein are methods of treating pancreatic cancer, said methods comprising, consisting of, or consisting essentially of, administering a therapeutically effective amount of an FGFR inhibitor generally, and erdafitinib more specifically, to a patient who has been diagnosed with pancreatic cancer and who harbors at least one FGFR genetic alteration, wherein the at least one FGFR genetic alteration is an FGFR fusion, in particular a FGFR1 fusion or a FGFR2 fusion. In certain embodiments, the at least one FGFR fusion is selected from FGFR1-MTUS1, FGFR2-ATAD2, FGFR2-CIT, FGFR2-GKAP1, FGFR2-GPHN, FGFR2-KCTD1, FGFR2-KIF6, FGFR2-NRBF2, FGFR2-ALDH1L1, and FGFR2-KIAA1598. In certain embodiments, the at least one FGFR fusion is selected from FGFR1-MTUS1, FGFR2-ATAD2, FGFR2-CIT, FGFR2-GKAP1, FGFR2-GPHN, FGFR2-KCTD1, FGFR2-KIF6, FGFR2-NRBF2, FGFR2-ALDH1L1, FGFR2-KIAA1598, and FGFR2-PAWR. In an embodiment, the at least one FGFR genetic alteration is an FGFR fusion, in particular a FGFR1 fusion or a FGFR2 fusion. In certain embodiments, the at least one FGFR fusion is selected from FGFR1-MTUS1, FGFR2-ATAD2, FGFR2-CIT, FGFR2-GKAP1, FGFR2-GPHN, FGFR2-KCTD1, FGFR2-KIF6, FGFR2-NRBF2, and FGFR2-PTEN. In an embodiment, the at least one FGFR fusion is FGFR1-MTUS1. In an embodiment, the at least one FGFR fusion is FGFR2-ATAD2. In an embodiment, the at least one FGFR fusion is FGFR2-CIT. In an embodiment, the at least one FGFR fusion is FGFR2-GKAP1. In an embodiment, the at least one FGFR fusion is FGFR2-GPHN. In an embodiment, the at least one FGFR fusion is FGFR2-KCTD1. In an embodiment, the at least one FGFR fusion is FGFR2-KIF6. In an embodiment, the at least one FGFR fusion is FGFR2-NRBF2. In an embodiment, the at least one FGFR fusion is FGFR2-PTEN. In an embodiment, the at least one FGFR fusion is FGFR2-ALDH1L1. In an embodiment, the at least one FGFR fusion is FGFR2-KIAA1598. In an embodiment, the at least one FGFR fusion is FGFR2-PAWR.

Also described herein are methods of treating pancreatic cancer, said methods comprising, consisting of, or consisting essentially of, administering a therapeutically effective amount of an FGFR inhibitor generally, and erdafitinib more specifically, to a patient who has been diagnosed with pancreatic cancer and who harbors at least one FGFR genetic alteration, wherein the at least one FGFR genetic alteration is an FGFR mutation. In an embodiment, the FGFR mutation is an FGFR1, FGFR2, or FGFR3 mutation.

Also described herein are methods of treating squamous NSCLC, said methods comprising, consisting of, or consisting essentially of, administering a therapeutically effective amount of an FGFR inhibitor generally, and erdafitinib more specifically, to a patient who has been diagnosed with squamous NSCLC and who harbors at least one FGFR genetic alteration, wherein the at least one FGFR genetic alteration is an FGFR fusion, in particular a FGFR3 fusion or an FGFR2 fusion. In certain embodiments the at least one FGFR fusion is selected from FGFR3-TACC3, FGFR3-TACC2, and WDR11-FGFR2. In certain embodiments, the at least one FGFR fusion is FGFR3-TACC3. In certain embodiments, the at least one FGFR fusion is FGFR2-TACC2. In certain embodiments, the at least one FGFR fusion is WDR11-FGFR2.

Also described herein are methods of treating squamous NSCLC, said methods comprising, consisting of, or consisting essentially of, administering a therapeutically effective amount of an FGFR inhibitor generally, and erdafitinib more specifically, to a patient who has been diagnosed with squamous NSCLC and who harbors at least one FGFR genetic alteration, wherein the at least one FGFR genetic alteration is an FGFR mutation, in particular a FGFR3 mutation. In certain embodiments the at least one FGFR mutation is selected from FGFR3-R248C and FGFR3-S249C. In certain embodiments, the at least one FGFR mutation is FGFR3-R248C. In certain embodiments, the at least one FGFR mutation is FGFR3-S249C.

Also described herein are methods of treating non-squamous NSCLC, said methods comprising, consisting of, or consisting essentially of, administering a therapeutically effective amount of an FGFR inhibitor generally, and erdafitinib more specifically, to a patient who has been diagnosed with non-squamous NSCLC and who harbors at least one FGFR genetic alteration, wherein the at least one FGFR genetic alteration is an FGFR fusion, in particular a FGFR2 fusion or a FGFR3 fusion. In certain embodiments, the at least one FGFR fusion is selected from FGFR2-BICC1, FGFR3-TACC3, and FGFR2-CCDC102A. In certain embodiments, the at least one FGFR fusion is selected from FGFR2-BICC1, FGFR3-TACC3, FGFR2-CCDC102A, and FGFR2-TACC2. In an embodiment, the at least one FGFR fusion is FGFR2-BICC1. In an embodiment, the at least one FGFR fusion is FGFR3-TACC3. In an embodiment, the at least one FGFR fusion is FGFR2-CCDC102A. In an embodiment, the at least one FGFR fusion is FGFR2-TACC2.

Also described herein are methods of treating non-squamous NSCLC, said methods comprising, consisting of, or consisting essentially of, administering a therapeutically effective amount of an FGFR inhibitor generally, and erdafitinib more specifically, to a patient who has been diagnosed with non-squamous NSCLC and who harbors at least one FGFR genetic alteration, wherein the at least one FGFR genetic alteration is an FGFR mutation, in particular a FGFR2 mutation or a FGFR3 mutation. In certain embodiments the at least one FGFR mutation is selected from FGFR2-Y375C, FGFR3-R399C, and FGFR3-S249C. In certain embodiments, the at least one FGFR mutation is FGFR2-Y375C. In certain embodiments, the at least one FGFR mutation is FGFR3-R399C. In certain embodiments, the at least one FGFR mutation is FGFR3-S249C.

Also described herein are methods of treating breast cancer, said methods comprising, consisting of, or consisting essentially of, administering a therapeutically effective amount of an FGFR inhibitor generally, and erdafitinib more specifically, to a patient who has been diagnosed with breast cancer and who harbors at least one FGFR genetic alteration, wherein the at least one FGFR genetic alteration is an FGFR fusion, in particular a FGFR1 fusion or a FGFR2 fusion. In certain embodiments, the at least one FGFR fusion is selected from FGFR1-TACC1, FGFR1-WHSCIL1, FGFR2-FKBP15, FGFR2-TACC2, FGFR2-TBC1D4, FGFR2-TCERG1L, FGFR2-BICC1 and FGFR2-KIAA1598, or is selected from FGFR1-TACC1, FGFR1-WHSC1L1, FGFR2-FKBP15, FGFR2-TACC2, FGFR2-TBC1D4, and FGFR2-TCERG1L. In certain embodiments, the at least one FGFR fusion is selected from FGFR1-TACC1, WHSC1L1-FGFR1, FGFR2-FKBP15, FGFR2-TACC2, FGFR2-TBC1D4, FGFR2-TCERG1L, FGFR2-BICC1 FGFR2-KIAA1598, CD44-FGFR2 FGFR2-FAM24B. In an embodiment, the at least one FGFR fusion is FGFR1-TACC1. In an embodiment, the at least one FGFR fusion is FGFR1-WHSCIL1. In an embodiment, the at least one FGFR fusion is WHSC1L1-FGFR1. In an embodiment, the at least one FGFR fusion is FGFR2-FKBP15. In an embodiment, the at least one FGFR fusion is FGFR2-TACC2. In an embodiment, the at least one FGFR fusion is FGFR2-TBC1D4. In an embodiment, the at least one FGFR fusion is FGFR2-TCERG1L. In an embodiment, the at least one FGFR fusion is FGFR2-BICC1. In an embodiment, the at least one FGFR fusion is FGFR2-KIAA1598. In an embodiment, the at least one FGFR fusion is CD44-FGFR2. In an embodiment, the at least one FGFR fusion is FGFR2-FAM24B.

Also described herein are methods of treating breast cancer, said methods comprising, consisting of, or consisting essentially of, administering a therapeutically effective amount of an FGFR inhibitor generally, and erdafitinib more specifically, to a patient who has been diagnosed with breast cancer and who harbors at least one FGFR genetic alteration, wherein the at least one FGFR genetic alteration is an FGFR mutation, in particular a FGFR2 mutation or an FGFR3 mutation. In certain embodiments, the at least one FGFR mutation is selected from FGFR2-C382R, FGFR2-K659M, FGFR3-R248C, and FGFR3-Y375C, or is selected from FGFR2-C382R and FGFR2-K659M. In an embodiment, the at least one FGFR mutation is FGFR2-C382R. In an embodiment, the at least one FGFR mutation is FGFR2-K659M. In an embodiment, the at least one FGFR mutation is FGFR3-R248C. In an embodiment, the at least one FGFR mutation is FGFR3-Y375C.

Also described herein are methods of treating colorectal cancer, said methods comprising, consisting of, or consisting essentially of, administering a therapeutically effective amount of an FGFR inhibitor generally, and erdafitinib more specifically, to a patient who has been diagnosed with colorectal cancer and who harbors at least one FGFR genetic alteration, wherein the at least one FGFR genetic alteration is an FGFR fusion, in particular a FGFR2 fusion or FGFR3 fusion. In certain embodiments, the at least one FGFR fusion is selected from FGFR2-BICC1 and FGFR3-TACC3. In certain embodiments, the at least one FGFR fusion is FGFR3-TACC3. In certain embodiments, the at least one FGFR fusion is FGFR2-BICC1.

Also described herein are methods of treating colorectal cancer, said methods comprising, consisting of, or consisting essentially of, administering a therapeutically effective amount of an FGFR inhibitor generally, and erdafitinib more specifically, to a patient who has been diagnosed with colorectal cancer and who harbors at least one FGFR genetic alteration, wherein the at least one FGFR genetic alteration is an FGFR mutation, in particular a FGR2 mutation or a FGFR3 mutation. In certain embodiments, the at least one FGFR mutation is selected from FGFR2-L770V, FGFR3-A500T, and FGFR3-F384L. In an embodiment, the at least one FGFR mutation is FGFR2-L770V. In an embodiment, the at least one FGFR mutation is FGFR3-A500T. In an embodiment, the at least one FGFR mutation is FGFR3-F384L.

Also described herein are methods of treating endometrial cancer, said methods comprising, consisting of, or consisting essentially of, administering a therapeutically effective amount of an FGFR inhibitor generally, and erdafitinib more specifically, to a patient who has been diagnosed with endometrial cancer and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating endometrial cancer, said methods comprising, consisting of, or consisting essentially of, administering a therapeutically effective amount of an FGFR inhibitor generally, and erdafitinib more specifically, to a patient who has been diagnosed with endometrial cancer and who harbors at least one FGFR genetic alteration, wherein the at least one FGFR genetic alteration is an FGFR mutation, in particular a FGFR1 or FGFR2 mutation. In certain embodiments, the at least one FGFR mutation is selected from FGFR2-C382R, FGFR2-D101Y, FGFR2-L551F, and FGFR2-Y375C. In certain embodiments, the at least one FGFR mutation is selected from FGFR1-S125L, FGFR2-C382R, FGFR2-D101Y, FGFR2-L551F, and FGFR2-Y375C. In an embodiment, the at least one FGFR mutation is FGFR1-S125L. In an embodiment, the at least one FGFR mutation is FGFR2-C382R. In an embodiment, the at least one FGFR mutation is FGFR2-C382R. In an embodiment, the at least one FGFR mutation is FGFR2-D101Y. In an embodiment, the at least one FGFR mutation is FGFR2-L551F. In an embodiment, the at least one FGFR mutation is FGFR2-Y375C.

Also described herein are methods of treating gastric cancer, said methods comprising, consisting of, or consisting essentially of, administering a therapeutically effective amount of an FGFR inhibitor generally, and erdafitinib more specifically, to a patient who has been diagnosed with gastric cancer and who harbors at least one FGFR genetic alteration, wherein the at least one FGFR genetic alteration is an FGFR fusion, in particular an FGFR3 fusion or an FGFR2 fusion. In certain embodiments, the at least one FGFR fusion is selected from FGFR3-TACC3 and FGFR2-HTRA1. In certain embodiments, the at least one FGFR fusion is FGFR3-TACC3. In certain embodiments, the FGFR fusion is FGFR2-HTRA1.

Also described herein are methods of treating gastric cancer, said methods comprising, consisting of, or consisting essentially of, administering a therapeutically effective amount of an FGFR inhibitor generally, and erdafitinib more specifically, to a patient who has been diagnosed with gastric cancer and who harbors at least one FGFR genetic alteration, wherein the at least one FGFR genetic alteration is an FGFR mutation, in particular a FGFR2 mutation or a FGFR3 mutation. In certain embodiments, the at least one FGFR mutation is selected from FGFR2-Y375C, FGFR3-S249C, and FGFR3-A500T. In an embodiment, the at least one FGFR mutation is FGFR2-Y375C. In an embodiment, the at least one FGFR mutation is FGFR3-S249C. In an embodiment, the at least one FGFR mutation is FGFR3-A500T.

Also described herein are methods of treating ovarian cancer, said methods comprising, consisting of, or consisting essentially of, administering a therapeutically effective amount of an FGFR inhibitor generally, and erdafitinib more specifically, to a patient who has been diagnosed with ovarian cancer and who harbors at least one FGFR genetic alteration, wherein the at least one FGFR genetic alteration is an FGFR fusion, in particular a FGFR1 fusion or a FGFR2 fusion. In certain embodiments, the at least one FGFR fusion is selected from FGFR1-RHPN2, FGFR2-AGAP1, and FGFR2-CLOCK. In certain embodiments, the at least one FGFR fusion is selected from RHPN2-FGFR1, FGFR2-AGAP1, and FGFR2-CLOCK. In an embodiment, the at least one FGFR fusion is FGFR1-RHPN2. In an embodiment, the at least one FGFR fusion is RHPN2-FGFR1. In an embodiment, the at least one FGFR fusion is FGFR2-AGAP1. In an embodiment, the at least one FGFR fusion is FGFR2-CLOCK.

Also described herein are methods of treating ovarian cancer, said methods comprising, consisting of, or consisting essentially of, administering a therapeutically effective amount of an FGFR inhibitor generally, and erdafitinib more specifically, to a patient who has been diagnosed with ovarian cancer and who harbors at least one FGFR genetic alteration, wherein the at least one FGFR genetic alteration is an FGFR mutation, in particular a FGFR3 mutation. In certain embodiments, the at least one FGFR mutation is FGFR3-S249C.

Also described herein are methods of treating carcinoma of unknown primary origin, said methods comprising, consisting of, or consisting essentially of, administering a therapeutically effective amount of an FGFR inhibitor generally, and erdafitinib more specifically, to a patient who has been diagnosed with carcinoma of unknown primary origin and who harbors at least one FGFR genetic alteration, wherein the at least one FGFR genetic alteration is an FGFR fusion, in particular a FGFR2 fusion. In certain embodiments, the at least one FGFR fusion is selected from FGFR2-TBC1D5 and FGFR2-BICC1. In certain embodiments, the at least one FGFR fusion is selected from FGFR2-TBC1D5, FGFR2-BICC1, FGFR2-CTNND2, and FGFR2-YPEL5. In an embodiment, the at least one FGFR fusion is FGFR2-TBC1D5. In an embodiment, the at least one FGFR fusion is FGFR2-BICC1. In an embodiment, the at least one FGFR fusion is FGFR2-CTNND2. In an embodiment, the at least one FGFR fusion is FGFR2-YPEL5.

Also described herein are methods of treating carcinoma of unknown primary origin, said methods comprising, consisting of, or consisting essentially of, administering a therapeutically effective amount of an FGFR inhibitor generally, and erdafitinib more specifically, to a patient who has been diagnosed with carcinoma of unknown primary origin and who harbors at least one FGFR genetic alteration, wherein the at least one FGFR genetic alteration is an FGFR mutation, in particular a FGFR2 mutation or a FGFR3 mutation. In certain embodiments, the at least one FGFR mutation is selected from FGFR3-S249C, FGFR2-S267P, and FGFR2-Y375C. In an embodiment, the at least one FGFR mutation is FGFR3-S249C. In an embodiment, the at least one FGFR mutation is FGFR2-S267P. In an embodiment, the at least one FGFR mutation is FGFR2-Y375C.

Also described herein are methods of treating cervical cancer, said methods comprising, consisting of, or consisting essentially of, administering a therapeutically effective amount of an FGFR inhibitor generally, and erdafitinib more specifically, to a patient who has been diagnosed with cervical cancer and who harbors at least one FGFR genetic alteration, wherein the at least one FGFR genetic alteration is an FGFR fusion. In an embodiment, the FGFR fusion is an FGFR1 fusion, and FGFR2 fusion, or an FGFR3 fusion. In an embodiment, the FGFR fusion is an FGFR3 fusion. In an embodiment, the at least one FGFR mutation is FGFR3-TACC3.

Also described herein are methods of treating cervical cancer, said methods comprising, consisting of, or consisting essentially of, administering a therapeutically effective amount of an FGFR inhibitor generally, and erdafitinib more specifically, to a patient who has been diagnosed with cervical cancer and who harbors at least one FGFR genetic alteration, wherein the at least one FGFR genetic alteration is an FGFR mutation, in particular a FGFR3 mutation. In certain embodiments, the at least one FGFR mutation is FGFR3-S249C.

Also described herein are methods of treating squamous cell head and neck cancer, said methods comprising, consisting of, or consisting essentially of, administering a therapeutically effective amount of an FGFR inhibitor generally, and erdafitinib more specifically, to a patient who has been diagnosed with squamous cell head and neck cancer and who harbors at least one FGFR genetic alteration, wherein the at least one FGFR genetic alteration is an FGFR fusion, in particular a FGFR3 fusion. In certain embodiments, the at least one FGFR fusion is FGFR3-TACC3.

Also described herein are methods of treating squamous cell head and neck cancer, said methods comprising, consisting of, or consisting essentially of, administering a therapeutically effective amount of an FGFR inhibitor generally, and erdafitinib more specifically, to a patient who has been diagnosed with squamous cell head and neck cancer and who harbors at least one FGFR genetic alteration, wherein the at least one FGFR genetic alteration is an FGFR mutation, in particular a FGFR3 mutation. In certain embodiments, the at least one FGFR mutation is selected from FGFR3-S249C and FGFR3-S371G. In an embodiment, the at least one FGFR mutation is FGFR3-S249C. In an embodiment, the at least one FGFR mutation is FGFR3-S371G.

Also described herein are methods of treating esophageal cancer, said methods comprising, consisting of, or consisting essentially of, administering a therapeutically effective amount of an FGFR inhibitor generally, and erdafitinib more specifically, to a patient who has been diagnosed with esophageal cancer and who harbors at least one FGFR genetic alteration, wherein the at least one FGFR genetic alteration is an FGFR fusion, in particular a FGFR3 fusion. In certain embodiments, the at least one FGFR fusion is selected from FGFR3-JAKMIP1 and FGFR3-TACC3. In certain embodiments, the at least one FGFR fusion is FGFR3-TACC3. In certain embodiments, the at least one FGFR fusion is FGFR3-JAKMIP1.

Also described herein are methods of treating esophageal cancer, said methods comprising, consisting of, or consisting essentially of, administering a therapeutically effective amount of an FGFR inhibitor generally, and erdafitinib more specifically, to a patient who has been diagnosed with esophageal cancer and who harbors at least one FGFR genetic alteration, wherein the at least one FGFR genetic alteration is an FGFR mutation, in particular a FGFR3 mutation. In certain embodiments, the at least one FGFR mutation is FGFR3-R248C. In certain embodiments, the at least one FGFR mutation is FGFR3-A500T.

Also described herein are methods of treating low-grade glioma, said methods comprising, consisting of, or consisting essentially of, administering a therapeutically effective amount of an FGFR inhibitor generally, and erdafitinib more specifically, to a patient who has been diagnosed with low-grade glioma and who harbors at least one FGFR genetic alteration, wherein the at least one FGFR genetic alteration is an FGFR fusion, in particular a FGFR3 fusion, an FGFR2 fusion, or an FGFR1 fusion. In certain embodiments, the at least one FGFR fusion is selected from FGFR1-TACC1, FGFR2-VPS35, and FGFR3-TACC3. In certain embodiments, the at least one FGFR fusion is FGFR3-TACC3. In certain embodiments, the at least one FGFR fusion is FGFR2-VPS35. In certain embodiments, the at least one FGFR fusion is FGFR1-TACC1.

Also described herein are methods of treating low-grade glioma, said methods comprising, consisting of, or consisting essentially of, administering a therapeutically effective amount of an FGFR inhibitor generally, and erdafitinib more specifically, to a patient who has been diagnosed with low-grade glioma and who harbors at least one FGFR genetic alteration, wherein the at least one FGFR genetic alteration is an FGFR mutation, in particular a FGFR1 mutation. In certain embodiments, the at least one FGFR mutation is FGFR1-K656E.

Also described herein are methods of treating prostate cancer, said methods comprising, consisting of, or consisting essentially of, administering a therapeutically effective amount of an FGFR inhibitor generally, and erdafitinib more specifically, to a patient who has been diagnosed with prostate cancer and who harbors at least one FGFR genetic alteration, wherein the at least one FGFR genetic alteration is an FGFR fusion, in particular a FGFR3 fusion. In certain embodiments, the at least one FGFR fusion is FGFR3-WHSC1. In certain embodiments, the at least one FGFR fusion is WHSC1-FGFR3.

Also described herein are methods of treating prostate cancer, said methods comprising, consisting of, or consisting essentially of, administering a therapeutically effective amount of an FGFR inhibitor generally, and erdafitinib more specifically, to a patient who has been diagnosed with prostate cancer and who harbors at least one FGFR genetic alteration, wherein the at least one FGFR genetic alteration is an FGFR mutation, in particular a FGFR3 mutation. In certain embodiments, the at least one FGFR mutation is FGFR3-R248C.

Also described herein are methods of treating salivary gland cancer, said methods comprising, consisting of, or consisting essentially of, administering a therapeutically effective amount of an FGFR inhibitor generally, and erdafitinib more specifically, to a patient who has been diagnosed with salivary gland cancer, and who harbors at least one FGFR genetic alteration, wherein the at least one FGFR genetic alteration is an FGFR fusion, in particular a FGFR1 fusion. In certain embodiments, the at least one FGFR fusion is FGFR1-PLAG1. In certain embodiments, the at least one FGFR genetic alteration is an FGFR mutation, in particular a FGFR2 mutation. In certain embodiments, the at least one FGFR mutation is FGFR2-C382R. In certain embodiments, the at least one FGFR genetic alteration is an FGFR fusion and an FGFR mutation. In certain embodiments, the FGFR fusion and FGFR mutation is FGFR1-PLAG1 and FGFR2-C382R. Also described herein are methods of treating salivary gland cancer, said methods comprising, consisting of, or consisting essentially of, administering a therapeutically effective amount of an FGFR inhibitor generally, and erdafitinib more specifically, to a patient who has been diagnosed with salivary gland cancer, and who harbors at least one FGFR genetic alteration, wherein the at least one FGFR genetic alteration is an FGFR mutation, in particular a FGFR2 mutation. In certain embodiments, the at least one FGFR mutation is selected from FGFR2-C382R, FGFR2-F276C and FGFR2-Y375C. In certain embodiments, the at least one FGFR mutation is selected from FGFR2-C382R, FGFR2-E565A, FGFR2-F276C, FGFR2-W72C, and FGFR2-Y375C. In an embodiment, the at least one FGFR mutation is FGFR2-C382R. In an embodiment, the at least one FGFR mutation is FGFR2-F276C. In an embodiment, the at least one FGFR mutation is FGFR2-Y375C. In an embodiment, the at least one FGFR mutation is FGFR2-E565A. In an embodiment, the at least one FGFR mutation is FGFR2-W72C. In an embodiment, the at least one FGFR mutation is FGFR2-E565A and FGFR2-W72C.

Also described herein are methods of treating basal cell carcinoma, said methods comprising, consisting of, or consisting essentially of, administering a therapeutically effective amount of an FGFR inhibitor generally, and erdafitinib more specifically, to a patient who has been diagnosed with basal cell carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating basal cell carcinoma, said methods comprising, consisting of, or consisting essentially of, administering a therapeutically effective amount of an FGFR inhibitor generally, and erdafitinib more specifically, to a patient who has been diagnosed with basal cell carcinoma, and who harbors at least one FGFR genetic alteration, wherein the at least one FGFR genetic alteration is an FGFR mutation, in particular a FGFR2 mutation. In certain embodiments, the at least one FGFR mutation is FGFR2-S252L.

Also described herein are methods of treating is thymic cancer, said methods comprising, consisting of, or consisting essentially of, administering a therapeutically effective amount of an FGFR inhibitor generally, and erdafitinib more specifically, to a patient who has been diagnosed with is thymic cancer, and who harbors at least one FGFR genetic alteration, wherein the at least one FGFR genetic alteration is an FGFR fusion, in particular a FGFR1 fusion. In certain embodiments, the at least one FGFR fusion is IGSF3-FGFR1.

Also described herein are methods of treating is thymic cancer, said methods comprising, consisting of, or consisting essentially of, administering a therapeutically effective amount of an FGFR inhibitor generally, and erdafitinib more specifically, to a patient who has been diagnosed with is thymic cancer, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating is small intestine adenocarcinoma, said methods comprising, consisting of, or consisting essentially of, administering a therapeutically effective amount of an FGFR inhibitor generally, and erdafitinib more specifically, to a patient who has been diagnosed with small intestine adenocarcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating small intestine adenocarcinoma, said methods comprising, consisting of, or consisting essentially of, administering a therapeutically effective amount of an FGFR inhibitor generally, and erdafitinib more specifically, to a patient who has been diagnosed with small intestine adenocarcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating is hepatocellular carcinoma, said methods comprising, consisting of, or consisting essentially of, administering a therapeutically effective amount of an FGFR inhibitor generally, and erdafitinib more specifically, to a patient who has been diagnosed with hepatocellular carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating hepatocellular carcinoma, said methods comprising, consisting of, or consisting essentially of, administering a therapeutically effective amount of an FGFR inhibitor generally, and erdafitinib more specifically, to a patient who has been diagnosed with hepatocellular carcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating is microcystic adnexal carcinoma, said methods comprising, consisting of, or consisting essentially of, administering a therapeutically effective amount of an FGFR inhibitor generally, and erdafitinib more specifically, to a patient who has been diagnosed with microcystic adnexal carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating microcystic adnexal carcinoma, said methods comprising, consisting of, or consisting essentially of, administering a therapeutically effective amount of an FGFR inhibitor generally, and erdafitinib more specifically, to a patient who has been diagnosed with microcystic adnexal carcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating is spinocellular carcinoma, said methods comprising, consisting of, or consisting essentially of, administering a therapeutically effective amount of an FGFR inhibitor generally, and erdafitinib more specifically, to a patient who has been diagnosed with spinocellular carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating spinocellular carcinoma, said methods comprising, consisting of, or consisting essentially of, administering a therapeutically effective amount of an FGFR inhibitor generally, and erdafitinib more specifically, to a patient who has been diagnosed with spinocellular carcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating gastrointestinal stromal tumor, said methods comprising, consisting of, or consisting essentially of, administering a therapeutically effective amount of an FGFR inhibitor generally, and erdafitinib more specifically, to a patient who has been diagnosed with gastrointestinal stromal tumor, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating gastrointestinal stromal tumor, said methods comprising, consisting of, or consisting essentially of, administering a therapeutically effective amount of an FGFR inhibitor generally, and erdafitinib more specifically, to a patient who has been diagnosed with gastrointestinal stromal tumor, and who harbors at least one FGFR genetic alteration, wherein the at least one FGFR genetic alteration is an FGFR mutation, in particular a FGFR3 mutation. In certain embodiments, the at least one FGFR mutation is FGFR3-S249F.

Also described herein are methods of treating parathyroid carcinoma, said methods comprising, consisting of, or consisting essentially of, administering a therapeutically effective amount of an FGFR inhibitor generally, and erdafitinib more specifically, to a patient who has been diagnosed with parathyroid carcinoma, and who harbors at least one FGFR genetic alteration, wherein the at least one FGFR genetic alteration is an FGFR fusion, in particular a FGFR1 fusion. In certain embodiments, the at least one FGFR fusion is FGFR1-BAG4. In certain embodiments, the at least one FGFR fusion is BAG4-FGFR1

Also described herein are methods of treating parathyroid carcinoma, said methods comprising, consisting of, or consisting essentially of, administering a therapeutically effective amount of an FGFR inhibitor generally, and erdafitinib more specifically, to a patient who has been diagnosed with parathyroid carcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating soft tissue sarcoma, said methods comprising, consisting of, or consisting essentially of, administering a therapeutically effective amount of an FGFR inhibitor generally, and erdafitinib more specifically, to a patient who has been diagnosed with soft tissue sarcoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion, in particular an FGFR1 fusion. In certain embodiments, the at least one FGFR fusion is FGFR1-MTUS1.

Also described herein are methods of treating soft tissue sarcoma, said methods comprising, consisting of, or consisting essentially of, administering a therapeutically effective amount of an FGFR inhibitor generally, and erdafitinib more specifically, to a patient who has been diagnosed with soft tissue sarcoma, and who harbors at least one FGFR genetic alteration, wherein the at least one FGFR genetic alteration is an FGFR mutation, in particular a FGFR1 mutation. In certain embodiments, the at least one FGFR mutation is FGFR1-K656E

Also described herein are methods of treating cup-syndrome, said methods comprising, consisting of, or consisting essentially of, administering a therapeutically effective amount of an FGFR inhibitor generally, and erdafitinib more specifically, to a patient who has been diagnosed with cup-syndrome, and who harbors at least one FGFR genetic alteration, wherein the at least one FGFR genetic alteration is an FGFR fusion, in particular a FGFR2 fusion. In certain embodiments, the at least one FGFR fusion is FGFR2-BICC1.

Also described herein are methods of treating cup-syndrome, said methods comprising, consisting of, or consisting essentially of, administering a therapeutically effective amount of an FGFR inhibitor generally, and erdafitinib more specifically, to a patient who has been diagnosed with cup-syndrome, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation

Also described herein are methods of treating anal adenocarcinoma, said methods comprising, consisting of, or consisting essentially of, administering a therapeutically effective amount of an FGFR inhibitor generally, and erdafitinib more specifically, to a patient who has been diagnosed with anal adenocarcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating anal adenocarcinoma, said methods comprising, consisting of, or consisting essentially of, administering a therapeutically effective amount of an FGFR inhibitor generally, and erdafitinib more specifically, to a patient who has been diagnosed with anal adenocarcinoma, and who harbors at least one FGFR genetic alteration, wherein the at least one FGFR genetic alteration is an FGFR mutation, in particular a FGFR3 mutation. In certain embodiments, the at least one FGFR mutation is FGFR3-R428C.

Also described herein are methods of treating anal adenoid cystic carcinoma, said methods comprising, consisting of, or consisting essentially of, administering a therapeutically effective amount of an FGFR inhibitor generally, and erdafitinib more specifically, to a patient who has been diagnosed with adenoid cystic carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating adenoid cystic carcinoma, said methods comprising, consisting of, or consisting essentially of, administering a therapeutically effective amount of an FGFR inhibitor generally, and erdafitinib more specifically, to a patient who has been diagnosed with anal adenoid cystic carcinoma, and who harbors at least one FGFR genetic alteration, wherein the at least one FGFR genetic alteration is an FGFR mutation, in particular a FGFR2 mutation. In certain embodiments, the at least one FGFR mutation is FGFR2-P253L.

Also described herein are methods of treating conjunctival epidermoid carcinoma, said methods comprising, consisting of, or consisting essentially of, administering a therapeutically effective amount of an FGFR inhibitor generally, and erdafitinib more specifically, to a patient who has been diagnosed with conjunctival epidermoid carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating conjunctival epidermoid carcinoma, said methods comprising, consisting of, or consisting essentially of, administering a therapeutically effective amount of an FGFR inhibitor generally, and erdafitinib more specifically, to a patient who has been diagnosed with conjunctival epidermoid carcinoma, and who harbors at least one FGFR genetic alteration, wherein the at least one FGFR genetic alteration is an FGFR mutation, in particular a FGFR3 mutation. In certain embodiments, the at least one FGFR mutation is FGFR3-S294C.

Also described herein are methods of treating duodenal cancer, said methods comprising, consisting of, or consisting essentially of, administering a therapeutically effective amount of an FGFR inhibitor generally, and erdafitinib more specifically, to a patient who has been diagnosed with duodenal cancer, and who harbors at least one FGFR genetic alteration, wherein the at least one FGFR genetic alteration is an FGFR fusion, in particular a FGFR2 fusion. In certain embodiments, the at least one FGFR fusion is FGFR2-TACC2.

Also described herein are methods of treating duodenal cancer, said methods comprising, consisting of, or consisting essentially of, administering a therapeutically effective amount of an FGFR inhibitor generally, and erdafitinib more specifically, to a patient who has been diagnosed with duodenal cancer, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating gallbladder carcinoma, said methods comprising, consisting of, or consisting essentially of, administering a therapeutically effective amount of an FGFR inhibitor generally, and erdafitinib more specifically, to a patient who has been diagnosed with gallbladder carcinoma, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating gallbladder carcinoma, said methods comprising, consisting of, or consisting essentially of, administering a therapeutically effective amount of an FGFR inhibitor generally, and erdafitinib more specifically, to a patient who has been diagnosed with gallbladder carcinoma, and who harbors at least one FGFR genetic alteration, wherein the at least one FGFR genetic alteration is an FGFR mutation, in particular a FGFR2 mutation. In certain embodiments, the at least one FGFR mutation is FGFR2-Y375C.

Also described herein are methods of treating germ cell tumor, said methods comprising, consisting of, or consisting essentially of, administering a therapeutically effective amount of an FGFR inhibitor generally, and erdafitinib more specifically, to a patient who has been diagnosed with germ cell tumor, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating germ cell tumor, said methods comprising, consisting of, or consisting essentially of, administering a therapeutically effective amount of an FGFR inhibitor generally, and erdafitinib more specifically, to a patient who has been diagnosed with germ cell tumor, and who harbors at least one FGFR genetic alteration, wherein the at least one FGFR genetic alteration is an FGFR mutation, in particular a FGFR3 mutation. In certain embodiments, the at least one FGFR mutation is FGFR3-P250R.

Also described herein are methods of treating mesothelioma, said methods comprising, consisting of, or consisting essentially of, administering a therapeutically effective amount of an FGFR inhibitor generally, and erdafitinib more specifically, to a patient who has been diagnosed with mesothelioma, and who harbors at least one FGFR genetic alteration, wherein the at least one FGFR genetic alteration is an FGFR fusion, in particular a FGFR2 fusion. In certain embodiments, the at least one FGFR fusion is FGFR2-GOLGA2.

Also described herein are methods of treating mesothelioma, said methods comprising, consisting of, or consisting essentially of, administering a therapeutically effective amount of an FGFR inhibitor generally, and erdafitinib more specifically, to a patient who has been diagnosed with mesothelioma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating malignant small round cell tumor, said methods comprising, consisting of, or consisting essentially of, administering a therapeutically effective amount of an FGFR inhibitor generally, and erdafitinib more specifically, to a patient who has been diagnosed with malignant small round cell tumor, and who harbors at least one FGFR genetic alteration, wherein the at least one FGFR genetic alteration is an FGFR fusion. In an embodiment, the FGFR fusion is an FGFR1 fusion, an FGFR2 fusion, or an FGFR3 fusion.

Also described herein are methods of treating malignant small round cell tumor, said methods comprising, consisting of, or consisting essentially of, administering a therapeutically effective amount of an FGFR inhibitor generally, and erdafitinib more specifically, to a patient who has been diagnosed with malignant small round cell tumor, and who harbors at least one FGFR genetic alteration, wherein the at least one FGFR genetic alteration is an FGFR mutation, in particular a FGFR3 mutation. In certain embodiments, the at least one FGFR mutation is FGFR3-S249C.

Also described herein are methods of treating testicular cancer, said methods comprising, consisting of, or consisting essentially of, administering a therapeutically effective amount of an FGFR inhibitor generally, and erdafitinib more specifically, to a patient who has been diagnosed with testicular cancer, and who harbors at least one FGFR genetic alteration, wherein the at least one FGFR genetic alteration is an FGFR fusion, in particular a FGFR3 fusion. In certain embodiments, the at least one FGFR fusion is FGFR3-TACC3.

Also described herein are methods of treating testicular cancer, said methods comprising, consisting of, or consisting essentially of, administering a therapeutically effective amount of an FGFR inhibitor generally, and erdafitinib more specifically, to a patient who has been diagnosed with testicular cancer, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating thyroid carcinoma, said methods comprising, consisting of, or consisting essentially of, administering a therapeutically effective amount of an FGFR inhibitor generally, and erdafitinib more specifically, to a patient who has been diagnosed with thyroid carcinoma, and who harbors at least one FGFR genetic alteration, wherein the at least one FGFR genetic alteration is an FGFR fusion, in particular a FGFR2 fusion. In certain embodiments, the at least one FGFR fusion is FGFR3-SENP6.

Also described herein are methods of treating thyroid carcinoma, said methods comprising, consisting of, or consisting essentially of, administering a therapeutically effective amount of an FGFR inhibitor generally, and erdafitinib more specifically, to a patient who has been diagnosed with thyroid carcinoma, and who harbors at least one FGFR genetic alteration, wherein the at least one FGFR genetic alteration is an FGFR mutation. In an embodiment, the FGFR mutation is an FGFR1 mutation, an FGFR2 mutation, or an FGFR3 mutation.

Also described herein are methods of treating cancer, said methods comprising, consisting of, or consisting essentially of, administering a therapeutically effective amount of an FGFR inhibitor, in particular erdafitinib, to a pediatric patient who has been diagnosed with cancer and who harbors at least one FGFR genetic alteration, wherein the cancer is glioblastoma multiforme, low grade glioma, pilocytic astrocytoma, rhabdomyosarcoma, Wilms' tumor, neuroblastoma, Ewing sarcoma, or medulloblastoma. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein are methods of treating glioblastoma multiforme, said methods comprising, consisting of, or consisting essentially of, administering a therapeutically effective amount of an FGFR inhibitor, in particular erdafitinib, to a pediatric patient who has been diagnosed with glioblastoma multiforme and who harbors at least one FGFR genetic alteration. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein is the use of an FGFR inhibitor, in particular erdafitinib, for the manufacture of a medicament for the treatment of a pediatric patient who has been diagnosed with cancer and who harbors at least one FGFR genetic alteration, wherein the cancer is glioblastoma multiforme, low grade glioma, pilocytic astrocytoma, rhabdomyosarcoma, Wilms' tumor, neuroblastoma, Ewing sarcoma, or medulloblastoma. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein is the use of an FGFR inhibitor, in particular erdafitinib, for the manufacture of a medicament for the treatment of a pediatric patient who has been diagnosed with glioblastoma multiforme and who harbors at least one FGFR genetic alteration. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein is a FGFR inhibitor, in particular erdafitinib, for use in the treatment of cancer in a pediatric patient who harbors at least one FGFR genetic alteration, wherein the cancer is glioblastoma multiforme, low grade glioma, pilocytic astrocytoma, rhabdomyosarcoma, Wilms' tumor, neuroblastoma, Ewing sarcoma, or medulloblastoma. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein is a FGFR inhibitor, in particular erdafitinib, for use in the treatment of glioblastoma multiforme in a pediatric patient who harbors at least one FGFR genetic alteration. In certain embodiments, the patient is ≥6 to <18 years of age. In certain embodiments, the patient is ≥6 to <12 years of age. In certain embodiments, the patient is ≥12 to <15 years of age. In certain embodiments, the patient is ≥15 to <18 years of age. In certain embodiments, the at least one FGFR genetic alteration is an FGFR mutation or an FGFR fusion, in particular an FGFR mutation or an FGFR fusion with an intact FGFR kinase domain.

Also described herein are methods of improving objective response rate in a patient or a population of patients with cancer relative to a comparative population of patients with cancer that is not receiving treatment with an FGFR inhibitor generally, and erdafitinib specifically, said method comprising providing to said patient or said population of patients a therapeutically effective amount of an FGFR inhibitor generally, and erdafitinib specifically. In certain embodiments, the objective response rate is assessed by an independent review committee. Objective response rate may be determined for an individual or a population of patients. In certain embodiments, the objective response rate, specifically the objective response rate assessed by an independent review committee, for the population of patients with cancer is about 29%. In certain embodiments, the objective response rate, specifically the objective response rate assessed by an independent review committee, for the population of patients with cancer is about 29.2%. In certain embodiments, the objective response rate, specifically the objective response rate assessed by an independent review committee, for the population of patients with cancer is at least about 29%. In certain embodiments, the objective response rate, specifically the objective response rate assessed by an independent review committee, for the population of patients with cancer is at least about 22%. In certain embodiments, the objective response rate, specifically the objective response rate assessed by an independent review committee, for the population of patients with cancer is about 22%, 23%, 24%, 25%, 26%, 27%, 28%, 29%, 30%, 31%, 32%, 33%, 34%, 35%, or 36%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer. Also described herein are methods of treating cancer, said methods comprising, consisting of, or consisting essentially of, administering a therapeutically effective amount of an FGFR inhibitor generally, and erdafitinib more specifically, to a patient population who has been diagnosed with an advanced solid tumor and who harbors target FGFR mutations or fusions and who has progressed on or after a minimum of 1 line of systemic therapy and for whom there are no remaining therapeutic options with established clinical benefit and wherein the objective response rate in said patient population is as described above. In certain embodiments, the patient was unable to tolerate standard of care therapies for the underlying tumor type.

Also described herein are methods of improving objective response rate in a patient or a population of patients with cancer who harbors at least one FGFR gene fusion relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the objective response rate is assessed by an independent review committee. Objective response rate may be determined for an individual or a population of patients. In certain embodiments, the objective response rate, specifically the objective response rate assessed by an independent review committee, for the population of patients with cancer is about 31.3%. In certain embodiments, the objective response rate, specifically the objective response rate assessed by an independent review committee, for the population of patients with cancer is about 30%. In certain embodiments, the objective response rate, specifically the objective response rate assessed by an independent review committee, for the population of patients with cancer is at least about 30%. In certain embodiments, the objective response rate, specifically the objective response rate assessed by an independent review committee, for the population of patients with cancer is about 31%. In certain embodiments, the objective response rate, specifically the objective response rate assessed by an independent review committee, for the population of patients with cancer is at least about 31%. In certain embodiments, the objective response rate, specifically the objective response rate assessed by an independent review committee, for the population of patients with cancer is about 22%. In certain embodiments, the objective response rate, specifically the objective response rate assessed by an independent review committee, for the population of patients with cancer is about 22%, 23%, 24%, 25%, 26%, 27%, 28%, 29%, 30%, 31%, 32%, 33%, 34%, 35%, or 36%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer.

Also described herein are methods of improving objective response rate in a patient or a population of patients with cancer who harbors at least one FGFR gene mutation relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the objective response rate is assessed by an independent review committee. Objective response rate may be determined for an individual or a population of patients. In certain embodiments, the objective response rate, specifically the objective response rate assessed by an independent review committee, for the population of patients with cancer is about 25.7%. In certain embodiments, the objective response rate, specifically the objective response rate assessed by an independent review committee, for the population of patients with cancer is about 26%. In certain embodiments, the objective response rate, specifically the objective response rate assessed by an independent review committee, for the population of patients with cancer is at least about 25.7%. In certain embodiments, the objective response rate, specifically the objective response rate assessed by an independent review committee, for the population of patients with cancer is about 26.8%. In certain embodiments, the objective response rate, specifically the objective response rate assessed by an independent review committee, for the population of patients with cancer is about 27%. In certain embodiments, the objective response rate, specifically the objective response rate assessed by an independent review committee, for the population of patients with cancer is at least about 22%. In certain embodiments, the objective response rate, specifically the objective response rate assessed by an independent review committee, for the population of patients with cancer is at least about 26%. In certain embodiments, the objective response rate, specifically the objective response rate assessed by an independent review committee, for the population of patients with cancer is about 22%, 23%, 24%, 25%, 26%, 27%, 28%, 29%, 30%, 31%, 32%, 33%, 34%, 35%, or 36%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer.

Also described herein are methods of improving objective response rate in a patient or a population of patients with cancer who harbors at least one FGFR gene fusion relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the objective response rate is investigator-assessed. Objective response rate may be determined for an individual or a population of patients. In certain embodiments, the objective response rate, specifically the investigator-assessed median duration of response, for the population of patients with cancer is at least about 26.4%. In certain embodiments, the objective response rate, specifically the investigator-assessed objective response rate, for the population of patients with cancer is at least about 26%. In certain embodiments, the objective response rate, specifically the investigator-assessed objective response rate, for the population of patients with cancer is at least about 22%. In certain embodiments, the objective response rate, specifically the investigator-assessed objective response rate, for the population of patients with cancer is about 22%, 23%, 24%, 25%, 26%, 27%, 28%, 29%, 30%, 31%, 32%, 33%, 34%, 35%, or 36%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of improving median duration of response in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the median duration of response is assessed by an independent review committee. Median duration of response may be determined for an individual or a population of patients. In certain embodiments, the median duration of response, specifically the median duration of response assessed by an independent review committee, for the population of patients with cancer is about 6.90 months. In certain embodiments, the median duration of response, specifically the median duration of response assessed by an independent review committee, for the population of patients with cancer is about 6.93 months. In certain embodiments, the median duration of response, specifically the median duration of response assessed by an independent review committee, for the population of patients with cancer is at least about 6.93 months. In certain embodiments, the median duration of response, specifically the median duration of response assessed by an independent review committee, for the population of patients with cancer is about 6.9 months. In certain embodiments, the median duration of response, specifically the median duration of response assessed by an independent review committee, for the population of patients with cancer is at least about 6.9 months. In certain embodiments, the median duration of response, specifically the median duration of response assessed by an independent review committee, for the population of patients with cancer is at least about 5.0 months. In certain embodiments, the median duration of response is about 5.0 months, 5.1 months, 5.2 months, 5.3 months, 5.4 months, 5.5 months, 5.6 months, at least about 5.7 months, 5.8 months, 5.9 months, 6.0 months, 6.1 months, 6.2 months, 6.3 months, 6.4 months, 6.5 months, 6.6 months, 6.7 months, 6.8 months, 6.9 months, 7.0 months, 7.1 months, 7.2 months, 7.3 months, 7.4 months, 7.5 months, 7.6 months, 7.8 months, 7.9 months, 8.0 months. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer.

Also described herein are methods of improving median duration of response in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the median duration of response is investigator-assessed. Median duration of response may be determined for an individual or a population of patients. In certain embodiments, the median duration of response, specifically the investigator-assessed median duration of response, for the population of patients with cancer is about 7.1 months. In certain embodiments, the median duration of response, specifically the investigator-assessed median duration of response, for the population of patients with cancer is about 7 months. In certain embodiments, the median duration of response, specifically the investigator-assessed median duration of response, for the population of patients with cancer is at least about 7 months. In certain embodiments, the median duration of response, specifically the investigator-assessed median duration of response, for the population of patients with cancer is at least about 5.0 months. In certain embodiments, the median duration of response is about 5.0 months, 5.1 months, 5.2 months, 5.3 months, 5.4 months, 5.5 months, 5.6 months, at least about 5.7 months, 5.8 months, 5.9 months, 6.0 months, 6.1 months, 6.2 months, 6.3 months, 6.4 months, 6.5 months, 6.6 months, 6.7 months, 6.8 months, 6.9 months, 7.0 months, 7.1 months, 7.2 months, 7.3 months, 7.4 months, 7.5 months, 7.6 months, 7.8 months, 7.9 months, 8.0 months. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of treating cancer, said methods comprising, consisting of, or consisting essentially of, administering a therapeutically effective amount of an FGFR inhibitor generally, and erdafitinib more specifically, to a patient population who has been diagnosed with an advanced solid tumor and who harbors target FGFR mutations or fusions and who has progressed on or after a minimum of 1 line of systemic therapy and for whom there are no remaining therapeutic options with established clinical benefit and wherein the median duration of response in said patient population is as described above. In certain embodiments, the patient was unable to tolerate standard of care therapies for the underlying tumor type.

Also described herein are methods of improving disease control rate in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the disease control rate is assessed by an independent review committee. Disease control rate may be determined for an individual or a population of patients. In certain embodiments, the disease control rate, specifically the disease control rate assessed by an independent review committee, for the population of patients with cancer is about 72.5%. In certain embodiments, the disease control rate, specifically the disease control rate assessed by an independent review committee, for the population of patients with cancer is at least about 72% or at least about 72.5%. In certain embodiments, the disease control rate, specifically the disease control rate assessed by an independent review committee, for the population of patients with cancer is about 77.4%. In certain embodiments, the disease control rate, specifically the disease control rate assessed by an independent review committee, for the population of patients with cancer is at least about 77.4%. In certain embodiments, the disease control rate, specifically the disease control rate assessed by an independent review committee, for the population of patients with cancer is at least about 75%. In certain embodiments, the disease control rate, specifically the disease control rate assessed by an independent review committee, for the population of patients with cancer is 72.0%, 72.5%, 73%, 73.5%, 74%, 74.5%, 75%, 75.5%, 76%, 76.6%, 77%, 77.7% or 78%. In certain embodiments, the disease control rate, specifically the disease control rate assessed by an independent review committee, for the population of patients with cancer is 75.0%, 75.1%, 75.2%, 75.3%, 75.4%, 75.5%, 75.6%, 75.7%, 75.8%, 75.9%, 76.0%, 76.1%, 76.2%, 76.3%, 76.4%, 76.5%, 76.6%, 76.7%, 76.8%, 76.9%, 77.0%, 77.1%, 77.2%, 77.3%, 77.4%, 77.5%, 77.6%, 77.7%, 77.8%, 77.9%, 78.0%, 78.1%, 78.2%, 78.3%, 78.4%, 78.5%, 78.6%, 78.7%, 78.8%, 78.9%, or 79.0%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer. Also described herein are methods of treating cancer, said methods comprising, consisting of, or consisting essentially of, administering a therapeutically effective amount of an FGFR inhibitor generally, and erdafitinib more specifically, to a patient population who has been diagnosed with an advanced solid tumor and who harbors target FGFR mutations or fusions and who has progressed on or after a minimum of 1 line of systemic therapy and for whom there are no remaining therapeutic options with established clinical benefit and wherein the disease control rate in said patient population is as described above. In certain embodiments, the patient was unable to tolerate standard of care therapies for the underlying tumor type.

Also described herein are methods of improving median time to response in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the disease control rate is assessed by an independent review committee. Median time to response may be determined for an individual or a population of patients. In certain embodiments, the median time to response, specifically the median time to response assessed by an independent review committee, for the population of patients with cancer is at least about 1 month. In certain embodiments, the median time to response, specifically the median time to response assessed by an independent review committee, for the population of patients with cancer is about 1.4 months. In certain embodiments, the median time to response, specifically the median time to response assessed by an independent review committee, for the population of patients with cancer is about 1.0 months, 1.1 months, 1.2 months, 1.3 months, 1.4 months, 1.5 months, 1.6 months, 1.7 months, 1.8 months, 1.9 months, or 2.0 months. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In certain embodiments, the cancer is cholangiocarcinoma, high-grade glioma, pancreatic cancer, breast cancer, squamous non-small-cell lung cancer (NSCLC), colorectal cancer, endometrial cancer, gastric cancer, ovarian cancer, squamous cell head and neck cancer, cervical cancer, low-grade glioma, non-squamous NSCLC, esophageal cancer, carcinoma of unknown primary origin, prostate cancer, salivary gland cancer, basocellular carcinoma, gastrointestinal stromal tumor, parathyroid carcinoma, or thymic cancer. Also described herein are methods of treating cancer, said methods comprising, consisting of, or consisting essentially of, administering a therapeutically effective amount of an FGFR inhibitor generally, and erdafitinib more specifically, to a patient population who has been diagnosed with an advanced solid tumor and who harbors target FGFR mutations or fusions and who has progressed on or after a minimum of 1 line of systemic therapy and for whom there are no remaining therapeutic options with established clinical benefit and wherein the median time to respond in said patient population is as described above. In certain embodiments, the patient was unable to tolerate standard of care therapies for the underlying tumor type.

Also described herein are methods of improving clinical benefit rate in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the clinical benefit rate is assessed by an independent review committee. Clinical benefit rate may be determined for an individual or a population of patients. In certain embodiments, the clinical benefit rate, specifically the clinical benefit rate assessed by an independent review committee, for the population of patients with cancer is about 46.1%. In certain embodiments, the clinical benefit rate, specifically the clinical benefit rate assessed by an independent review committee, for the population of patients with cancer is about 46%. In certain embodiments, the clinical benefit rate, specifically the clinical benefit rate assessed by an independent review committee, for the population of patients with cancer is at least about 46%. In certain embodiments, the clinical benefit rate, specifically the clinical benefit rate assessed by an independent review committee, for the population of patients with cancer is about 40% or is at least about 40%. In certain embodiments, the clinical benefit rate, specifically the clinical benefit rate assessed by an independent review committee, for the population of patients with cancer is 40%, 41%, 42%, 43%, 44%, 45%, 46%, 47%, 48%, 49% or 50%. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of improving median progression free survival in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the median progression free survival is assessed by an independent review committee. Median progression free survival may be determined for an individual or a population of patients. In certain embodiments, the median progression free survival, specifically the median progression free survival assessed by an independent review committee, for the population of patients with cancer is about 4.2 months. In certain embodiments, the median progression free survival, specifically the median progression free survival assessed by an independent review committee, for the population of patients with cancer is about 4 months. In certain embodiments, the median progression free survival, specifically the median progression free survival assessed by an independent review committee, for the population of patients with cancer is at least about 4 months. In certain embodiments, the median progression free survival, specifically the median progression free survival assessed by an independent review committee, for the population of patients with cancer is at least about 3 months. In certain embodiments, the median progression free survival, specifically the median progression free survival assessed by an independent review committee, for the population of patients with cancer is 3 months, 3.5 months, 4 months, or 4.5 months. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein are methods of improving median overall survival in a patient or a population of patients with cancer relative to a comparative population of patients with cancer that is not receiving treatment with an FGFR inhibitor generally, and erdafitinib specifically, said method comprising providing to said patient a therapeutically effective amount of an FGFR inhibitor generally, and erdafitinib specifically. In certain embodiments, the median overall survival is assessed by an independent review committee. Median overall survival may be determined for an individual or a population of patients. In certain embodiments, the median overall survival, specifically the median overall survival assessed by an independent review committee, for the population of patients with cancer is about 10.94 months. In certain embodiments, the median overall survival, specifically the median overall survival assessed by an independent review committee, for the population of patients with cancer is about 11 months. In certain embodiments, the median overall survival, specifically the median overall survival assessed by an independent review committee, for the population of patients with cancer is at least about 11 months. In certain embodiments, the median overall survival, specifically the median overall survival assessed by an independent review committee, for the population of patients with cancer is at least about 9 months. In certain embodiments, the median overall survival, specifically the median overall survival assessed by an independent review committee, for the population of patients with cancer is 9 months, 9.5 months, 10 months, 10.5 months, 11 months, 11.5 months, or 12 months. In certain embodiments, the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

In certain embodiments, administration of the FGFR inhibitor generally, and erdafitinib specifically, provides improved anti-tumor activity as measured by objective response rate, median duration of response, disease control rate, median time to response, clinical benefit rate, progression-free survival, or overall survival relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by objective response rate relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by median duration of response relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by disease control rate relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by median time to response relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by clinical benefit rate relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by progression-free survival relative to a comparative population of patients with cancer that is not receiving treatment with an FGFR inhibitor generally, and erdafitinib specifically. In certain embodiments, administration of the FGFR inhibitor generally, and erdafitinib specifically, provides improved anti-tumor activity as measured by overall survival relative to a comparative population of patients with cancer that is not receiving treatment with an FGFR inhibitor generally, and erdafitinib specifically.

In any of the foregoing methods of treatment, in certain embodiments, also described herein are methods of treating cancer, said methods comprising, consisting of, or consisting essentially of, administering a therapeutically effective amount of an FGFR inhibitor generally, and erdafitinib more specifically, to a patient who has been diagnosed with an advanced solid tumor, in particular any tumor or list of tumors provided herein, and who harbors target FGFR mutations or fusions, in particular any FGFR mutation or fusion or list of FGFR mutations or fusions provided herein, and who has progressed on or after a minimum of 1 line of systemic therapy and for whom there are no remaining therapeutic options with established clinical benefit and wherein the objective response rate in said patient population is as described above. In certain embodiments, the patient was unable to tolerate standard of care therapies for the underlying tumor type.

In any of the foregoing methods of treatment, in certain embodiments, also described herein are methods of treating cancer, said methods comprising, consisting of, or consisting essentially of, administering a therapeutically effective amount of an FGFR inhibitor generally, and erdafitinib more specifically, to a patient who has been diagnosed with an advanced solid tumor, in particular any tumor or list of tumors provided herein, and who harbors target FGFR mutations or fusions, in particular any FGFR mutation or fusion or list of FGFR mutations or fusions provided herein, and who has progressed on or after at least one line of systemic therapy and for whom there are no effective alternative treatments.

In any of the foregoing methods of treatment, in certain embodiments, also described herein are methods of treating cancer, said methods comprising, consisting of, or consisting essentially of, administering a therapeutically effective amount of an FGFR inhibitor generally, and erdafitinib more specifically, to a patient who has been diagnosed with a locally advanced or metastatic solid tumor, in particular any tumor or list of tumors provided herein, and who harbors target FGFR mutations or fusions, in particular any FGFR mutation or fusion or list of FGFR mutations or fusions provided herein, and who has progressed following prior therapies and who have no acceptable standard therapies.

In certain embodiments, the improvement in anti-tumor activity is relative to standard of care. In certain embodiments, the improvement in anti-tumor activity is relative to no treatment with an FGFR inhibitor generally, and erdafitinib specifically.

In some embodiments, the patient or population of patients to whom the FGFR inhibitor is administered and the comparative population of patients with cancer that is not receiving treatment with an FGFR inhibitor generally, and erdafitinib specifically, both having previously been treated by the same or similar prior treatment regimen.

In certain embodiments, the patient population is defined as the patient population which completed the clinical trial detailed herein in the Examples. In certain embodiments, the patient is an adult. In certain embodiments, the patient is an adolescent, optionally aged 15 to <18 years. In certain embodiments, the patient is an adolescent, optionally aged 12 to <15 years. In certain embodiments the patient is a pediatric patient, optionally aged 6 to <12 years.

For each the methods of treatment described herein, it will be understood that the methods of treatment may also be framed as methods of manufacturing a medicament for the treatment of the described indications or as a use for the manufacture of a medicament for the treatment of the described indications or as an FGFR inhibitor generally, or erdafitinib specifically, for use in the treatment of the described indications. In any of the described embodiments, the FGFR fusion may by any FGFR fusion wherein the FGFR protein has an intact FGFR kinase domain. In certain embodiments, the FGFR fusion is a FGFR1 fusion, in particular a FGFR1 fusion as described herein. In certain embodiments, the FGFR fusion is a FGFR2 fusion, in particular a FGFR2 fusion as described herein. In certain embodiments, the FGFR fusion is a FGFR3 fusion, in particular a FGFR3 fusion as described herein. In certain embodiments, the FGFR fusion is a FGFR1 fusion, a FGFR2 fusion or a FGFR3 fusion, in particular a FGFR1 fusion, a FGFR2 fusion or FGFR3 fusion as described herein. In certain embodiments, the FGFR fusion is a FGFR2 fusion or a FGFR3 fusion, in particular a FGFR2 fusion or FGFR3 fusion as described herein. In certain embodiments, the FGFR mutation is a FGFR2 mutation, in particular a FGFR2 mutation as described herein. In certain embodiments, the FGFR mutation is a FGFR3 mutation, in particular a FGFR3 mutation as described herein. In certain embodiments, the FGFR mutation is a FGFR2 mutation or a FGFR3 mutation, in particular a FGFR2 mutation or a FGFR3 mutation as described herein. In certain embodiments, the indication is an advanced solid tumor with a FGFR1 fusion, in particular a FGFR1 fusion as described herein. In certain embodiments, the indication is an advanced solid tumor with a FGFR2 fusion, in particular a FGFR2 fusion as described herein. In certain embodiments, the indication is an advanced solid tumor with a FGFR3 fusion, in particular a FGFR3 fusion as described herein. In certain embodiments, the indication is an advanced solid tumor with a FGFR1 fusion, a FGFR2 fusion or a FGFR3 fusion, in particular a FGFR1 fusion, a FGFR2 fusion or FGFR3 fusion as described herein. In certain embodiments, the indication is an advanced solid tumor with a FGFR2 fusion or a FGFR3 fusion, in particular a FGFR2 fusion or FGFR3 fusion as described herein. In certain embodiments, the indication is an advanced solid tumor with a FGFR2 mutation, in particular a FGFR2 mutation as described herein. In certain embodiments, the indication is an advanced solid tumor with a FGFR3 mutation, in particular a FGFR3 mutation as described herein. In certain embodiments, the indication is an advanced solid tumor with a FGFR2 mutation or a FGFR3 mutation, in particular a FGFR2 mutation or a FGFR3 mutation as described herein.

In any of the described embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSC1L1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In any of the described embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCILI, FGFR2-AGAP1, FGFR2-AHCYL1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-PTEN, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In any of the described embodiments, the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSC1L1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-ENOX1, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In any of the described embodiments, the at least one FGFR genetic alteration is FGFR2-HTRA1, FGFR2-IMIPA1, FGFR2-CTNND2, FGFR2-YPEL5, FGFR2-SENP6, FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSC1L1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1.

In any of the described embodiments, the at least one FGFR genetic alteration is FGFR1-PLAG1, FGFR2-C382R, BAG4-FGFR1, IGSF3-FGFR1, FGFR1-K656E, FGFR1-MTUS1, RHPN2-FGFR1, FGFR1-TACC1, WHSC1L1-FGFR1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, WHSC1-FGFR3, CD44-FGFR2, FGFR2-CTNND2, FGFR2-FAM24B, FGFR2-GOLGA2, FGFR2-HTRA1, FGFR2-IMIPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, FGFR3-P250R, or FGFR3-R399C.

In any of the described embodiments, the at least one FGFR genetic alteration is FGFR1-MTUS1, FGFR1-PLAG1, FGFR1-TACC1, FGFR2-ATAD2, FGFR2-BICC1, FGFR2-CCDC102A, FGFR2-ENOX1, FGFR2-FKBP15, FGFR2-GKAP1, FGFR2-GPHN, FGFR2-KCTD1, FGFR2-KIAA1598, FGFR2-NOL4, FGFR2-PAWR, FGFR2-SENP6, FGFR2-TACC2, FGFR2-TBC1D4, FGFR2-TRA2B, FGFR2-VPS35, FGFR2-WAC, FGFR3-TACC3, FGFR1-K656E, FGFR2-C382R, FGFR2-E565A, FGFR2-F276C, FGFR2-W72C, FGFR2-Y375C, FGFR3-R248C, or FGFR3-S249C.

In certain embodiments, the subject received at least one line of systemic therapy prior to said administration of an FGFR inhibitor, in particular erdafitinib. The subject received at least one line of systemic therapy prior to said administration of an FGFR inhibitor, in particular erdafitinib, in the metastatic setting. In an embodiment, the subject progressed on or after at least one line of systemic therapy and for whom there are no further available therapies with established clinical benefit prior to said administration of an FGFR inhibitor, in particular erdafitinib. In an embodiment, the subject progressed on or after at least one line of systemic therapy prior to said administration of an FGFR inhibitor, in particular erdafitinib, and who are unable to tolerate standard therapies.

In certain embodiments, said methods or uses further comprise evaluating a biological sample from the patient for the presence of at least one of a FGFR fusion, in particular the at least one FGFR fusions as described herein, or at least one FGFR genetic alteration, in particular the at least one FGFR genetic alteration as described herein, prior to said administration of erdafitinib. In certain embodiments, the biological sample is blood, lymph fluid, bone marrow, a solid tumor sample, or any combination thereof. In certain embodiments, said methods or uses further comprise evaluating a biological sample from the patient for the presence of at least one of a FGFR mutation, in particular the at least one FGFR mutations as described herein, prior to said administration of erdafitinib. In certain embodiments, the biological sample is blood, lymph fluid, bone marrow, a solid tumor sample, or any combination thereof.

In certain embodiments, said methods or uses further comprise determining if the patient harbors at least one of a FGFR fusion, in particular the at least one FGFR fusions as described herein, or at least one FGFR genetic alteration, in particular the at least one FGFR genetic alteration as described herein, prior to said administration of erdafitinib. In certain embodiments, the biological sample is blood, lymph fluid, bone marrow, a solid tumor sample, or any combination thereof. In certain embodiments, said methods or uses further comprise determining if the patient harbors at least one of a FGFR mutation, in particular the at least one FGFR mutations as described herein, prior to said administration of erdafitinib. In certain embodiments, the biological sample is blood, lymph fluid, bone marrow, a solid tumor sample, or any combination thereof.

In some embodiments, the patient is 15 years of age or older at the date of first administration of the FGFR inhibitor. In certain embodiments, the patient is an adult of ≥18 years of age. In certain embodiments, the patient is an adolescent between 15 to <18 years of age. In further embodiments, the FGFR inhibitor, in particular erdafitinib, is administered daily, in particular once daily. In still further embodiments, the FGFR inhibitor, in particular erdafitinib, is administered orally. In certain embodiments, the FGFR inhibitor, in particular erdafitinib, is administered orally on a continuous daily dosing schedule. In some embodiments, erdafitinib is administered orally at a dose of about 8 mg once daily. As used herein, “between” is inclusive of the lower age range. For example, between 15 years of age and <18 years of age includes patients who are 15 years of age. Also as used herein, the upper age range includes patients up to the day before the patient turns the indicated age, e.g. 18 years of age. In an embodiment, erdafitinib is administered at a dose of 8 mg, in particular 8 mg once daily, with an option to up-titrate to 9 mg, depending on serum phosphate levels (e.g. serum phosphate levels are <7 mg/dL or range from and include 7 mg/dL to <9 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of the first cycle of erdafitinib administration. As used herein, day 14 after initiating treatment, day 14 of the first cycle of erdafitinib administration, Cycle 1 Day 14 and C1D14 are used interchangeably. In some embodiments, erdafitinib is administered orally at a dose of about 8 mg once daily on a continuous daily dosing schedule. In further embodiments, the dose of erdafitinib is increased from 8 mg per day to 9 mg per day after initiating treatment if the patient exhibits a serum phosphate (PO₄) level that is less than about 7.0 mg/dL, in particular the dose of erdafitinib is increased from 8 mg per day to 9 mg per day after initiating treatment if the patient exhibits a serum PO₄ level that is less than about 7.0 mg/dL at 14 days, optionally 14 plus 2 days, in particular at 14 days, after initiating treatment. In further embodiments, the dose of erdafitinib is increased from 8 mg per day to 9 mg per day after initiating treatment if the patient exhibits a serum PO₄ level that ranges from and including 7.0 mg/dL to <9.0 mg/dL, in particular the dose of erdafitinib is increased from 8 mg per day to 9 mg per day after initiating treatment if the patient exhibits a serum PO₄ level that ranges from and including 7.0 mg/dL to <9.0 mg/dL at 14 days, optionally 14 plus 2 days, in particular at 14 days, after initiating treatment. In certain embodiments, the increase in dose of erdafitinib from 8 mg to 9 mg is concomitant with administration of a phosphate binder, in particular if the patient exhibits a serum PO₄ level that ranges from and including 7.0 mg/dL to <9.0 mg/dL. In certain embodiments, the phosphate binder is sevelamer. In certain embodiments the 8 mg per day is 8 mg once daily. In certain embodiments, the 9 mg per day is 9 mg once daily.

In some embodiments, the patient is between 12 years of age and <15 years of age at the date of first administration of said FGFR inhibitor. As used herein, “between” is inclusive of the lower age range. For example, between 12 years of age and <15 years of age includes patients who are 12 years of age. Also as used herein, the upper age range includes patients up to the day before the patient turns the indicated age, e.g., 15 years of age. In an embodiment, erdafitinib is administered at a dose of 5 mg, in particular 5 mg once daily, with an option to up-titrate to 6 mg, in particular 6 mg once daily, and with a further option to up-titrate to 8 mg, in particular 8 mg once daily, depending on serum phosphate levels (e.g. serum phosphate levels are <7 mg/dL or range from and include 7.0 mg/dL to <9.0 mg/dL), and depending on treatment-related adverse events observed. In an embodiment, the levels of serum phosphate for determining whether or not to up-titrate, in particular to up-titrate from 5 mg once daily to 6 mg once daily, are measured on a treatment day during the first cycle of erdafitinib treatment, in particular on day 14 plus 2 days, more in particular on day 14 of the first cycle of erdafitinib administration. As used herein, day 14 after initiating treatment, day 14 of the first cycle of erdafitinib administration, Cycle 1 Day 14 and C1D14 are used interchangeably. The dose of erdafitinib is increased from 5 mg per day to 6 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 5 mg once daily to 6 mg once daily is concomitant with administration of a phosphate binder, in particular if the patient exhibits a serum PO₄ level that ranges from and including 7.0 mg/dL to <9.0 mg/dL. In an embodiment, the levels of serum phosphate for determining whether or not to up-titrate, in particular to up-titrate from 5 mg once daily to 6 mg once daily, are measured on a treatment day during the second cycle of erdafitinib treatment, in particular on day 7 of the second cycle of erdafitinib administration (Cycle 2 Day 7 or C2D7). The dose of erdafitinib is increased from 5 mg per day to 6 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 5 mg once daily to 6 mg once daily is concomitant with administration of a phosphate binder, in particular if the patient exhibits a serum PO₄ level that ranges from and including 7.0 mg/dL to <9.0 mg/dL. In an embodiment, the levels of serum phosphate for determining whether or not to up-titrate, in particular to up-titrate from 6 mg once daily to 8 mg once daily for those already up-titrated to 6 mg on C1D14 plus 2 days, more in particular C1D14, are measured on a treatment day during the second cycle of erdafitinib treatment, in particular on day 7 of the second cycle of erdafitinib administration (Cycle 2 Day 7 or C2D7). The dose of erdafitinib is increased from 6 mg per day to 8 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 6 mg once daily to 8 mg once daily is concomitant with administration of a phosphate binder, in particular if the patient exhibits a serum PO₄ level that ranges from and including 7.0 mg/dL to <9.0 mg/dL. In certain embodiments, the phosphate binder is sevelamer. In certain embodiments the 5 mg per day is 5 mg once daily. In certain embodiments, the 6 mg per day is 6 mg once daily. In certain embodiments, the 8 mg per day is 8 mg once daily.

In certain embodiments, erdafitinib is administered at a dose of about 5 mg once daily. In further embodiments, the dose of erdafitinib is increased from 5 mg per day to 6 mg per day after initiating treatment if the patient exhibits a serum phosphate (PO₄) level that ranges from and including 7.0 mg/dL to <9 mg/dL at 7 or 14 days, optionally 14 plus 2 days, in particular at 14 days, after initiating treatment. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at 7 or 14 days, optionally 14 plus 2 days, in particular at 14 days, after initiating treatment. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at day 7 of the second cycle of erdafitinib treatment. In further embodiments, the dose of erdafitinib is further increased from 6 mg per day to 8 mg per day after initiating treatment if the patient exhibits a serum PO₄ level that ranges from and including 7.0 to <9 mg/dL at 14 days, optionally 14 plus 2 days, in particular at 14 days, after initiating treatment. In certain embodiments, the dose of erdafitinib is further increased from 6 mg per day to 8 mg per day after initiating treatment if the patient exhibits a serum PO₄ level that ranges from and includes 7.0 to <9 mg/dL at day 7 of the second cycle of erdafitinib treatment. In certain embodiments, the increase in dose of erdafitinib from 5 mg to 6 mg or from 6 mg to 8 mg is concomitant with administration of a phosphate binder, in particular if the patient exhibits a serum PO₄ level that ranges from and including 7.0 mg/dL to <9.0 mg/dL at 14 days after initiating treatment or at day 7 of the second cycle of treatment. In certain embodiments, the phosphate binder is sevelamer. In still further embodiments, the dose of erdafitinib is increased from 5 mg per day to 6 mg per day after initiating treatment if the patient exhibits a serum PO₄ level of less than 7.0 mg/dL at 7 or 14 days, optionally 14 plus 2 days, in particular at 14 days, after initiating treatment. In still further embodiments, the dose of erdafitinib is increased from 5 mg per day to 6 mg per day after initiating treatment if the patient exhibits a serum P04 level of less than 7.0 mg/dL at 14 days, optionally 14 plus 2 days, in particular at 14 days, after initiating treatment. In still further embodiments, the dose of erdafitinib is increased from 5 mg per day to 6 mg per day after initiating treatment if the patient exhibits a serum PO₄ level of less than 7.0 mg/dL at day 7 of the second cycle of erdafitinib treatment In further embodiments, the dose of erdafitinib is further increased from 6 mg per day to 8 mg per day after initiating treatment if the patient exhibits a serum PO₄ level of less than 7.0 mg/dL at 14 days, optionally 14 plus 2 days, in particular at 14 days after initiating treatment. In further embodiments, the dose of erdafitinib is further increased from 6 mg per day to 8 mg per day after initiating treatment if the patient exhibits a serum PO₄ level of less than 7.0 mg/dL at day 7 of the second cycle of erdafitinib treatment. In further embodiments, the 2-step up-titration (from 5 mg to 6 mg, and from 6 mg to 8 mg) is step-wise, i.e. no subject is allowed to directly up-titrate from 5 mg to 8 mg.

In some embodiments, if the patient is between 6 years of age and <12 years of age at the date of first administration of said FGFR inhibitor, in particular erdafitinib, the FGFR inhibitor, in particular erdafitinib, is administered at a dose of about 3 mg, in particular 3 mg once daily. As used herein, “between” is inclusive of the lower age range. For example, between 6 years of age and <12 years of age includes patients who are 6 years of age. Also as used herein, the upper age range includes patients up to the day before the patient turns the indicated age, e.g. 12 years of age. In an embodiment, erdafitinib is administered at a dose of 3 mg, in particular 3 mg once daily, with an option to up-titrate to 4 mg, in particular 4 mg once daily, and with a further option to up-titrate from 4 mg to 5 mg, in particular 5 mg once daily, depending on serum phosphate levels (e.g. serum phosphate levels are <7 mg/dL or range from and include 7 mg/dL to <9 mg/dL, in particular 7.0 mg/dL to <9.0 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of the first cycle of erdafitinib administration. As used herein, day 14 after initiating treatment, day 14 of the first cycle of erdafitinib administration, Cycle 1 Day 14 and C1D14 are used interchangeably. In an embodiment, the levels of serum phosphate for determining whether or not to up-titrate are measured on a treatment day during the second cycle of erdafitinib treatment, in particular on day 7 of the second cycle of erdafitinib administration (Cycle 2 Day 7 or C2D7). In certain embodiments, erdafitinib is administered at a dose of about 3 mg once daily. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at 14 days, optionally 14 plus 2 days, after initiating treatment. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at day 7 of the second cycle of erdafitinib treatment. In certain embodiments, the dose of erdafitinib is further increased from 4 mg per day to 5 mg per day after initiating treatment if the patient exhibits a serum PO₄ level that ranges from and includes 7.0 to <9 mg/dL at day 7 of the second cycle of erdafitinib treatment. In certain embodiments, the increase in dose of erdafitinib from 3 mg to 4 mg or from 4 mg to 5 mg is in combination with administration of a phosphate binder, e.g. sevelamer, in particular if the patient exhibits a serum PO₄ level that ranges from and including 7.0 mg/dL to <9.0 mg/dL at 14 days after initiating treatment or at day 7 of the second cycle of treatment. In certain embodiments, the increase in dose of erdafitinib from 3 mg to 4 mg or from 4 mg to 5 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment or at day 7 of the second cycle of erdafitinib treatment. In still further embodiments, the dose of erdafitinib is increased from 3 mg per day to 4 mg per day after initiating treatment if the patient exhibits a serum P04 level of less than 7.0 mg/dL at 14 days, optionally 14 plus 2 days, in particular at 14 days, after initiating treatment. In still further embodiments, the dose of erdafitinib is increased from 3 mg per day to 4 mg per day after initiating treatment if the patient exhibits a serum PO₄ level of less than 7.0 mg/dL at day 7 of the second cycle of erdafitinib treatment. In further embodiments, the dose of erdafitinib is further increased from 4 mg per day to 5 mg per day after initiating treatment if the patient exhibits a serum PO₄ level of less than 7.0 mg/dL at day 7 of the second cycle of erdafitinib treatment. In further embodiments, the 2-step up-titration (from 3 mg to 4 mg, and from 4 mg to 5 mg) is stepwise, i.e., no subject is allowed to directly up-titrate from 3 mg to 5 mg. In certain embodiments the 3 mg per day is 3 mg once daily. In certain embodiments, the 4 mg per day is 4 mg once daily. In certain embodiments, the 5 mg per day is 5 mg once daily.

In some embodiments, the patient is between 6 years of age and <12 years of age at the date of first administration of said FGFR inhibitor. As used herein, “between” is inclusive of the lower age range. For example, between 6 years of age and <12 years of age includes patients who are 6 years of age. Also as used herein, the upper age range includes patients up to the day before the patient turns the indicated age, e.g., 12 years of age. In an embodiment, erdafitinib is administered at a dose of 3 mg, in particular 3 mg once daily, with an option to up-titrate to 4 mg, in particular 4 mg once daily, and with a further option to up-titrate to 5 mg, in particular 5 mg once daily, depending on serum phosphate levels (e.g. serum phosphate levels are <7 mg/dL or range from and include 7.0 mg/dL to <9.0 mg/dL), and depending on treatment-related adverse events observed. In an embodiment, the levels of serum phosphate for determining whether or not to up-titrate, in particular to up-titrate from 3 mg once daily to 4 mg once daily, are measured on a treatment day during the first cycle of erdafitinib treatment, in particular on day 14 plus 2 days, more in particular on day 14 of the first cycle of erdafitinib administration. As used herein, day 14 after initiating treatment, day 14 of the first cycle of erdafitinib administration, Cycle 1 Day 14 and C1D14 are used interchangeably. The dose of erdafitinib is increased from 3 mg per day to 4 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 3 mg once daily to 4 mg once daily is concomitant with administration of a phosphate binder, in particular if the patient exhibits a serum PO₄ level that ranges from and including 7.0 mg/dL to <9.0 mg/dL. In an embodiment, the levels of serum phosphate for determining whether or not to up-titrate, in particular to up-titrate from 3 mg once daily to 4 mg once daily, are measured on a treatment day during the second cycle of erdafitinib treatment, in particular on day 7 of the second cycle of erdafitinib administration (Cycle 2 Day 7 or C2D7). The dose of erdafitinib is increased from 3 mg per day to 4 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 3 mg once daily to 4 mg once daily is concomitant with administration of a phosphate binder, in particular if the patient exhibits a serum PO₄ level that ranges from and including 7.0 mg/dL to <9.0 mg/dL. In an embodiment, the levels of serum phosphate for determining whether or not to up-titrate, in particular to up-titrate from 4 mg once daily to 5 mg once daily for those already up-titrated to 4 mg on C1D14 plus 2 days, more in particular C1D14, are measured on a treatment day during the second cycle of erdafitinib treatment, in particular on day 7 of the second cycle of erdafitinib administration (Cycle 2 Day 7 or C2D7). The dose of erdafitinib is increased from 4 mg per day to 5 mg per day after initiating treatment if the patient exhibits a serum phosphate level of <7 mg/dL or that ranges from and include 7.0 mg/dL to <9.0 mg/dL. In certain embodiments, the increase in dose of erdafitinib from 4 mg once daily to 5 mg once daily is concomitant with administration of a phosphate binder, in particular if the patient exhibits a serum PO₄ level that ranges from and including 7.0 mg/dL to <9.0 mg/dL. In certain embodiments, the phosphate binder is sevelamer. In certain embodiments the 3 mg per day is 3 mg once daily. In certain embodiments, the 4 mg per day is 4 mg once daily. In certain embodiments, the 5 mg per day is 5 mg once daily.

In certain embodiments, erdafitinib is administered in a solid dosage form. In further embodiments, the solid dosage form is a tablet.

Treatment with erdafitinib should be discontinued or modified based on erdafitinib-related toxicity as described in Table A.

TABLE A
Erdafitinib dose modification rules based
on erdafitinib-related toxicity severity

| | No up-titration at | With up-titration at |
|---|---|---|
| | Cycle 1 Day 14 | Cycle 1 Day 14 |
| Category | Dose | Dose |
| Starting dose | 8 mg | 8 mg |
| Up-titration at Cycle 1 Day 14 | None | 9 mg |
| 1^{st} dose reduction | 6 mg | 8 mg |
| 2^{nd} dose reduction | 5 mg | 6 mg |
| 3^{rd} dose reduction | 4 mg | 5 mg |
| 4^{th} does reduction | stop | 4 mg |
| 5^{th} dose reduction | — | stop |

Subjects with any grade of toxicity (Grade 1 to 4) should be provided symptomatic treatment where applicable.

If erdafitinib is interrupted consecutively for 1 week or longer due to drug-related toxicity, the study drug may be reintroduced at either the same dose level or the first reduced dose level following recovery from the toxicity (see dose reduction levels in Table B, Table C, and Table D). A second dose reduction may be implemented following a second occurrence of drug-related toxicity.

If erdafitinib must be withheld for more than 28 days for a drug-related adverse event that fails to resolve to acceptable level (e.g., <Grade 1 non-hematologic toxicity or back to baseline), treatment with erdafitinib should be discontinued except when the subject has been deriving benefit from treatment, and the investigator can demonstrate that continued treatment with erdafitinib is in the best interest of the subject. Erdafitinib may be re-started at the same or a lower dose (Table B, Table C, and Table D) if the sponsor's medical monitor concurs with the assessment.

If erdafitinib was dose-reduced and the adverse event that was the reason for this dose-reduction has completely resolved, the dose may be re-escalated to the next higher dose if the subject was deriving benefit from treatment, and the investigator can demonstrate that dose re-escalation of erdafitinib is in the best interest of the subject and the medical monitor concurs with the assessment.

In all cases of clinically significant impaired wound healing or imminent surgery or potential bleeding complications, it is recommended that dose administration be interrupted, appropriate clinical laboratory data (e.g., coagulation parameters) be carefully monitored, and supportive therapy administered, where applicable. Dose administration may be restarted when it is considered safe and at an appropriate dose, according to the investigator's assessment.

TABLE B
Erdafitinib dose reduction levels: Adults and
Adolescents Aged ≥15 to <18 years

| | No up-titration at | With up-titration |
|---|---|---|
| | Cycle 1 Day 14 | at Cycle 1 Day 14 |
| Category | Dose | Dose |
| Starting dose | 8 mg | 8 mg |
| Up-titration at Cycle 1 Day 14 | None | 9 mg |
| 1^{st} dose reduction | 6 mg | 8 mg |
| 2^{nd} dose reduction | 5 mg | 6 mg |
| 3^{rd} dose reduction | 4 mg | 5 mg |
| 4^{th} does reduction | stop | 4 mg |
| 5^{th} dose reduction | — | stop |

TABLE C
Erdafitinib dose reduction levels: Adolescents Aged ≥12 to <15 years

| | | |
|---|---|---|
| | No up-titration at | With up-titration |
| Category | Cycle 1 Day 14 | at Cycle 1 Day 14 |

For Cycle 1 Day 14

| | | |
|---|---|---|
| Starting Dose on Cycle 1 | 5 mg | 5 mg |
| Day 1 | | |
| Up-titration at Cycle 1 | None | 6 mg |
| Day 14 | | |
| 1^{st} dose reduction | 4 mg | 5 mg |
| 2^{nd} dose reduction | 3 mg | 4 mg |
| 3^{rd} dose reduction | stop | 3 mg |
| 4^{th} does reduction | — | stop |

| | | | |
|---|---|---|---|
| | | No up-titration at | Up-titration at Cycle |
| | No up-titration at | Cycle 1 Day 14 but | 1 Day 14 followed by |
| | Cycle 1 Day 14 and | with up-titration at | up-titration at Cycle |
| Category | Cycle 2 Day 7 | Cycle 2 Day 7 | 2 Day 7 |

For Cycle 2 Day 7

| | | | |
|---|---|---|---|
| Dose of Cycle 2 Day 7 | 5 mg | 5 mg | 6 mg |
| Up-titration at Cycle | None | 6 mg | 8 mg |
| 2 Day 7 | | | |
| 1^{st} dose reduction | 4 mg | 5 mg | 6 mg |
| 2^{nd} dose reduction | 3 mg | 4 mg | 5 mg |
| 3^{rd} dose reduction | stop | 3 mg | 4 mg |
| 4^{th} dose reduction | — | stop | 3 mg |
| 5^{th} dose reduction | — | — | stop |

TABLE D
Erdafitinib dose reduction levels: Children Aged ≥6 to <12 years

| | | |
|---|---|---|
| | No up-titration | With up-titration |
| | at Cycle 1 | at Cycle 1 |
| Category | Day 14 | Day 14 |

For Cycle 1 Day 14

| | | |
|---|---|---|
| Starting Dose on Cycle 1 | 3 mg | 3 mg |
| Day 1 | | |
| Up-titration at Cycle 1 Day 14 | None | 4 mg |
| 1^{st} dose reduction | Stop | 3 mg |
| 2^{nd} dose reduction | — | stop |

| | | | |
|---|---|---|---|
| | | No up-titration at | Up-titration at Cycle |
| | No up-titration at | Cycle 1 Day 14 but | 1 Day 14 followed by |
| | Cycle 1 Day 14 and | with up-titration at | up-titration at Cycle |
| Category | Cycle 2 Day 7 | Cycle 2 Day 7 | 2 Day 7 |

For Cycle 2 Day 7

| | | | |
|---|---|---|---|
| Dose of Cycle 2 Day | 3 mg | 3 mg | 4 mg |
| 7 | | | |
| Up-titration at Cycle | None | 4 mg | 5 mg |
| 2 Day 7 | | | |
| 1^{st} dose reduction | Stop | 3 mg | 4 mg |
| 2^{nd} dose reduction | — | Stop | 3 mg |
| 3^{rd} dose reduction | — | — | Stop |

Described herein is an FGFR inhibitor, in particular erdafitinib, for use in the treatment of cancer in a patient who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. In certain embodiments the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. The FGFR inhibitor is to be administered at a therapeutically effective dose.

Also described herein is an FGFR inhibitor, in particular erdafitinib, for use in the treatment of cancer in a patient who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma. The FGFR inhibitor is to be administered at a therapeutically effective dose.

Also described herein is an FGFR inhibitor, in particular erdafitinib, for use in the treatment of cancer in a patient who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. The FGFR inhibitor is to be administered at a therapeutically effective dose.

Also described herein is an FGFR inhibitor, in particular erdafitinib, for use in the treatment of cancer in a patient who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma. The FGFR inhibitor is to be administered at a therapeutically effective dose.

Further described herein are uses of an FGFR inhibitor, in particular erdafitinib, for the manufacture of a medicament for the treatment of a patient who has been diagnosed with a cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. In certain embodiments the at least one FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. The FGFR inhibitor is to be administered at a therapeutically effective dose.

Still further described herein are uses of an FGFR inhibitor, in particular erdafitinib, for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma. The FGFR inhibitor is to be administered at a therapeutically effective dose.

Still further described herein are uses of an FGFR inhibitor, in particular erdafitinib, for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. The FGFR inhibitor is to be administered at a therapeutically effective dose.

Still further described herein are uses of an FGFR inhibitor, in particular erdafitinib, for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma. The FGFR inhibitor is to be administered at a therapeutically effective dose.

Still further described herein are uses of an FGFR inhibitor, in particular erdafitinib, for the manufacture of a medicament for the treatment of a patient who has been diagnosed with a cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. The FGFR inhibitor is to be administered at a therapeutically effective dose.

### Evaluating a Sample for the Presence of One or More FGFR Genetic Alterations

Described herein are methods of treating cancer comprising, consisting of, or consisting essentially of: evaluating a biological sample for the presence of at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2 from a patient who has been diagnosed with cancer; and administering a therapeutically effective dose of an FGFR inhibitor at to the patient if at least one FGFR fusion is present in the sample. In an embodiment, the FGFR inhibitor is erdafitinib.

Described herein are methods of treating cancer comprising, consisting of, or consisting essentially of: evaluating a biological sample for the presence of at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1 from a patient who has been diagnosed with cancer; and administering a therapeutically effective dose of an FGFR inhibitor at to the patient if at least one FGFR fusion is present in the sample. In an embodiment, the FGFR inhibitor is erdafitinib.

Also described herein are methods of treating cancer comprising, consisting of, or consisting essentially of: determining if a patient who has been diagnosed with cancer harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2; and administering a therapeutically effective dose of an FGFR inhibitor to the patient if the patient harbors at least one of the FGFR fusions. In an embodiment, the FGFR inhibitor is erdafitinib. Described herein is the use of an FGFR inhibitor, in particular erdafitinib, for the manufacture of a medicament for the treatment of a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2, and wherein erdafitinib is administered or is to be administered after evaluation of a biological sample from the patient for the presence at least one FGFR fusion and if one or more FGFR fusion is present in the sample.

Also described herein are methods of treating cancer comprising, consisting of, or consisting essentially of: determining if a patient who has been diagnosed with cancer harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1; and administering a therapeutically effective dose of an FGFR inhibitor to the patient if the patient harbors at least one of the FGFR fusions. In an embodiment, the FGFR inhibitor is erdafitinib. Described herein is the use of an FGFR inhibitor, in particular erdafitinib, for the manufacture of a medicament for the treatment of a patient who has been diagnosed with cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1, and wherein erdafitinib is administered or is to be administered after evaluation of a biological sample from the patient for the presence at least one FGFR fusion and if one or more FGFR fusion is present in the sample

Also described herein is an FGFR inhibitor, in particular erdafitinib, for use in the treatment of a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2, and wherein erdafitinib is administered or is to be administered after evaluation of a biological sample from the patient for the presence at least one FGFR fusion and if one or more FGFR fusion is present in the sample.

Also described herein is an FGFR inhibitor, in particular erdafitinib, for use in the treatment of a patient who has been diagnosed with cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1, and wherein erdafitinib is administered or is to be administered after evaluation of a biological sample from the patient for the presence at least one FGFR fusion and if one or more FGFR fusion is present in the sample

Also described herein are methods of treating cancer comprising, consisting of, or consisting essentially of: evaluating a biological sample for the presence of at least on FGFR gene alteration from a patient who has been diagnosed with cancer, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; and administering a therapeutically effective dose of an FGFR inhibitor to the patient if at least one FGFR alteration is present in the sample. In an embodiment, the FGFR inhibitor is erdafitinib.

Also described herein are methods of treating cancer comprising, consisting of, or consisting essentially of: evaluating a biological sample for the presence of at least on FGFR gene alteration from a patient who has been diagnosed with cancer, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR alteration is present in the sample. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In an embodiment, the FGFR inhibitor is erdafitinib.

Also described herein are methods of treating cancer comprising, consisting of, or consisting essentially of: evaluating a biological sample for the presence of at least on FGFR gene alteration from a patient who has been diagnosed with cancer, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma; and administering a therapeutically effective dose of an FGFR inhibitor to the patient if at least one FGFR alteration is present in the sample. In an embodiment, the FGFR inhibitor is erdafitinib.

Also described herein are methods of treating cancer comprising, consisting of, or consisting essentially of: evaluating a biological sample for the presence of at least on FGFR gene alteration from a patient who has been diagnosed with cancer, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR alteration is present in the sample. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In an embodiment, the FGFR inhibitor is erdafitinib.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma; and administering a therapeutically effective dose of an FGFR inhibitor to the patient if at least one FGFR gene alteration is present in the sample. In an embodiment, the FGFR inhibitor is erdafitinib.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR gene alteration is present in the sample. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In an embodiment, the FGFR inhibitor is erdafitinib.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma; and administering a therapeutically effective dose of an FGFR inhibitor to the patient if at least one FGFR gene alteration is present in the sample. In an embodiment, the FGFR inhibitor is erdafitinib.

Also described herein are methods of treating cancer comprising, consisting of, or consisting essentially of: determining if a patient who has been diagnosed with cancer harbors at least one FGFR gene alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma; and administering a therapeutically effective dose of an FGFR inhibitor to the patient if at least one FGFR gene alteration is present in the sample. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma. In an embodiment, the FGFR inhibitor is erdafitinib.

Described herein is the use of an FGFR inhibitor, in particular erdafitinib, for the manufacture of a medicament for the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

Described herein is the use of an FGFR inhibitor, in particular erdafitinib, for the manufacture of a medicament for the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Described herein is the use of an FGFR inhibitor, in particular erdafitinib, for the manufacture of a medicament for the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma.

Described herein is the use of an FGFR inhibitor, in particular erdafitinib, for the manufacture of a medicament for the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein is an FGFR inhibitor, in particular erdafitinib, for use in the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma.

Also described herein is an FGFR inhibitor, in particular erdafitinib, for use in the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

Also described herein is an FGFR inhibitor, in particular erdafitinib, for use in the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma.

Also described herein is an FGFR inhibitor, in particular erdafitinib, for use in the treatment of a patient who has been diagnosed with cancer and who harbors at least one FGFR fusion, and wherein erdafitinib is administered or is to be administered after evaluation of a biological sample from the patient for the presence at least one FGFR alteration and if one or more FGFR alteration is present in the sample, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma. In certain embodiments, the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma.

The following methods for evaluating a biological sample for the presence of one or more FGFR genetic alterations or determining if a patient harbors one or more FGFR genetic alterations apply equally to any of the above disclosed methods of treatment and uses.

The disclosed methods are suitable for treating cancer in a patient if one or more FGFR genetic alterations are present in a biological sample from the patient. In some embodiments, the FGFR genetic alteration can be one or more FGFR fusion genes, in particular one or more FGFR1, FGFR2 or FGFR3 fusion genes. In some embodiments, the FGFR genetic alteration can be one or more FGFR mutations, in particular one or more FGFR1, FGFR2 or FGFR3 mutations. In some embodiments, a combination of the one or more FGFR genetic alterations can be present in the biological sample from the patient. For example, in some embodiments, the FGFR genetic alterations can be one or more FGFR fusion genes and one or more FGFR mutations.

Exemplary FGFR alterations are provided in Table 9, Table 14 or Table 19 and include but are not limited to: FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCIL1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1, or a combination thereof.

In certain embodiments, exemplary FGFR alterations are provided in Table 9, Table 14 or Table 19 and include but are not limited to: FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCILl, FGFR2-AGAP1, FGFR2-AHCYL1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-PTEN, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1, or a combination thereof.

In certain embodiments, exemplary FGFR alterations are provided in Table 9, Table 14 or Table 19 and include but are not limited to: FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCIL1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-ENOX1, FGFR3-MYH14, FGFR3-R248C, FGFR3-S249C, FGFR3-S249F, FGFR3-S371G, FGFR3-TACC3, FGFR3-TMEM247, or FGFR3-WHSC1, or a combination thereof.

Exemplary FGFR alterations are provided in Table 9, Table 14 or Table 19 and include but are not limited to: FGFR2-HTRA1, FGFR2-IMPA1, FGFR2-CTNND2, FGFR2-YPEL5, FGFR2-SENP6, FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSCILl, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, or FGFR3-WHSC1, or a combination thereof.

Exemplary FGFR alterations are provided in Table 9, Table 14 or Table 19 and include but are not limited to: FGFR1-PLAG1, FGFR2-C382R, BAG4-FGFR1, IGSF3-FGFR1, FGFR1-K656E, FGFR1-MTUS1, RHPN2-FGFR1, FGFR1-TACC1, WHSCILl-FGFR1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, WHSC1-FGFR3, CD44-FGFR2, FGFR2-CTNND2, FGFR2-FAM24B, FGFR2-GOLGA2, FGFR2-HTRA1, FGFR2-IMPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, FGFR3-P250R, or FGFR3-R399C, or a combination thereof.

Exemplary FGFR alterations are provided in Table 9, Table 14 or Table 19 and include but are not limited to: FGFR1-MTUS1, FGFR1-PLAG1, FGFR1-TACC1, FGFR2-ATAD2, FGFR2-BICC1, FGFR2-CCDC102A, FGFR2-ENOX1, FGFR2-FKBP15, FGFR2-GKAP1, FGFR2-GPHN, FGFR2-KCTD1, FGFR2-KIAA1598, FGFR2-NOL4, FGFR2-PAWR, FGFR2-SENP6, FGFR2-TACC2, FGFR2-TBC1D4, FGFR2-TRA2B, FGFR2-VPS35, FGFR2-WAC, FGFR3-TACC3, FGFR1-K656E, FGFR2-C382R, FGFR2-E565A, FGFR2-F276C, FGFR2-W72C, FGFR2-Y375C, FGFR3-R248C, or FGFR3-S249C, or a combination thereof.

Suitable methods for evaluating a biological sample for the presence of one or more FGFR genetic alterations are described in the methods section herein and in WO 2016/048833 and U.S. patent application Ser. No. 16/723,975, which are incorporated herein in their entireties. For example, and without intent to be limiting, evaluating a biological sample for the presence of one or more FGFR genetic alterations can comprise any combination of the following steps: isolating RNA from the biological sample; synthesizing cDNA from the RNA; and amplifying the cDNA (pre-amplified or non-pre-amplified). In some embodiments, evaluating a biological sample for the presence of one or more FGFR genetic alterations can comprise: amplifying cDNA from the patient with a pair of primers that bind to and amplify one or more FGFR genetic alterations; and determining whether the one or more FGFR genetic alterations are present in the sample. In some aspects of the disclosure, the cDNA can be pre-amplified. In some aspects of the disclosure, the evaluating step can comprise isolating RNA from the sample, synthesizing cDNA from the isolated RNA, and pre-amplifying the cDNA.

The presence of one or more FGFR genetic alterations can be evaluated at any suitable time point including upon diagnosis, following tumor resection, following first-line therapy, during clinical treatment, or any combination thereof.

For example, a biological sample taken from a patient may be analyzed to determine whether a condition or disease, such as cancer, that the patient is or may be suffering from is one which is characterized by a genetic abnormality or abnormal protein expression which leads to up-regulation of the levels or activity of FGFR or to sensitization of a pathway to normal FGFR activity, or to upregulation of these growth factor signaling pathways such as growth factor ligand levels or growth factor ligand activity or to upregulation of a biochemical pathway downstream of FGFR activation.

Examples of such abnormalities that result in activation or sensitization of the FGFR signal include loss of, or inhibition of apoptotic pathways, up-regulation of the receptors or ligands, or presence of genetic alterations of the receptors or ligands e.g. PTK variants. Tumors with genetic alterations of FGFR1, FGFR2 or FGFR3 or FGFR4 or up-regulation, in particular over-expression of FGFR1, or gain-of-function genetic alterations of FGFR2 or FGFR3 may be particularly sensitive to FGFR inhibitors.

The methods, approved drug products, and uses can further comprise evaluating the presence of one or more FGFR genetic alterations in the biological sample before the administering step.

The diagnostic tests and screens are typically conducted on a biological sample selected from tumor biopsy samples, blood samples (isolation and enrichment of shed tumor cells), stool biopsies, sputum, chromosome analysis, pleural fluid, peritoneal fluid, buccal spears, biopsy, circulating DNA, or urine. In certain embodiments, the biological sample is blood, lymph fluid, bone marrow, a solid tumor sample, or any combination thereof. In certain embodiments, the biological sample is a solid tumor sample. In certain embodiments, the biological sample is a blood sample. In certain embodiments, the biological sample is a urine sample.

Methods of identification and analysis of genetic alterations and up-regulation of proteins are known to a person skilled in the art. Screening methods could include, but are not limited to, standard methods such as reverse-transcriptase polymerase chain reaction (RT PCR) or in-situ hybridization such as fluorescence in situ hybridization (FISH).

Identification of an individual carrying a genetic alteration in FGFR, in particular an FGFR genetic alteration as described herein, may mean that the patient would be particularly suitable for treatment with an FGFR inhibitor, in particular erdafitinib. Tumors may preferentially be screened for presence of a FGFR variant prior to treatment. The screening process will typically involve direct sequencing, oligonucleotide microarray analysis, or a mutant specific antibody. In addition, diagnosis of tumor with such genetic alteration could be performed using techniques known to a person skilled in the art and as described herein such as RT-PCR, FISH, or next-generation sequencing.

In addition, genetic alterations of, for example FGFR, can be identified by direct sequencing of, for example, tumor biopsies using PCR and methods to sequence PCR products directly as hereinbefore described. The skilled artisan will recognize that all such well-known techniques for detection of the over expression, activation or mutations of the aforementioned proteins could be applicable in the present case.

In screening by RT-PCR, the level of mRNA in the tumor is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. (2004) Current Protocols in Molecular Biology, John Wiley & Sons Inc., or Innis, M. A. et al., eds. (1990) PCR Protocols: a guide to methods and applications, Academic Press, San Diego. Reactions and manipulations involving nucleic acid techniques are also described in Sambrook et al., (2001), 3rd Ed, Molecular Cloning: A Laboratory Manual, Cold Spring Harbor Laboratory Press. Alternatively, a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference. An example of an in-situ hybridization technique for assessing mRNA expression would be fluorescence in-situ hybridization (FISH) (see Angerer (1987) Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. (2004) Current Protocols in Molecular Biology, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Methods for gene expression profiling are described by (DePrimo et al. (2003), BMC Cancer, 3:3). Briefly, the protocol is as follows: double-stranded cDNA is synthesized from total RNA Using a (dT)24 oligomer for priming first-strand cDNA synthesis, followed by second strand cDNA synthesis with random hexamer primers. The double-stranded cDNA is used as a template for in vitro transcription of cRNA using biotinylated ribonucleotides. cRNA is chemically fragmented according to protocols described by Affymetrix (Santa Clara, CA, USA), and then hybridized overnight on Human Genome Arrays.

Alternatively, the protein products expressed from the mRNAs may be assayed by immunohistochemistry of tumor samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site-specific antibodies. The skilled person will recognize that all such well-known techniques for detection of upregulation of FGFR or detection of FGFR variants or mutants could be applicable in the present case.

Abnormal levels of proteins such as FGFR can be measured using standard enzyme assays, for example, those assays described herein. Activation or overexpression could also be detected in a tissue sample, for example, a tumor tissue. By measuring the tyrosine kinase activity with an assay such as that from Chemicon International. The tyrosine kinase of interest would be immunoprecipitated from the sample lysate and its activity measured.

Alternative methods for the measurement of the over expression or activation of FGFR including the isoforms thereof, include the measurement of microvessel density. This can for example be measured using methods described by Orre and Rogers (Int J Cancer (1999), 84(2) 101-8). Assay methods also include the use of markers.

Therefore, all of these techniques could also be used to identify tumors particularly suitable for treatment with the compounds of the invention.

### Pharmaceutical Compositions and Routes of Administration

In view of its useful pharmacological properties, the FGFR inhibitor generally, and erdafitinib more specifically, may be formulated into various pharmaceutical forms for administration purposes.

In one embodiment the pharmaceutical composition (e.g. formulation) comprises at least one FGFR inhibitor together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

To prepare the pharmaceutical compositions, an effective amount of the FGFR inhibitor generally, and erdafitinib more specifically, as the active ingredient is combined in intimate admixture with a pharmaceutically acceptable carrier, which carrier may take a wide variety of forms depending on the form of preparation desired for administration. The pharmaceutical compositions can be in any form suitable for oral, parenteral, topical, intranasal, ophthalmic, otic, rectal, intra-vaginal, or transdermal administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets.

The pharmaceutical compositions of the invention, in particular capsules and/or tablets, may include one or more pharmaceutically acceptable excipients (pharmaceutically acceptable carrier) such as disintegrants, diluents, fillers, binders, buffering agents, lubricants, glidants, thickening agents, sweetening agents, flavors, colorants, preservatives and the like. Some excipients can serve multiple purposes.

Suitable disintegrants are those that have a large coefficient of expansion. Examples thereof are hydrophilic, insoluble or poorly water-soluble crosslinked polymers such as crospovidone (crosslinked polyvinylpyrrolidone) and croscarmellose sodium (crosslinked sodium carboxymethylcellulose). The amount of disintegrant in the tablets according to the present invention may conveniently range from about 2.5 to about 15% w/w and preferably range from about 2.5 to 7% w/w, in particular range from about 2.5 to 5% w/w. Because disintegrants by their nature yield sustained release formulations when employed in bulk, it is advantageous to dilute them with an inert substance called a diluent or filler.

A variety of materials may be used as diluents or fillers. Examples are lactose monohydrate, anhydrous lactose, sucrose, dextrose, mannitol, sorbitol, starch, cellulose (e.g. micro-crystalline cellulose (Avicel™), silicified microcrystalline cellulose), dihydrated or anhydrous dibasic calcium phosphate, and others known in the art, and mixtures thereof (e.g. spray-dried mixture of lactose monohydrate (75%) with microcrystalline cellulose (25%) which is commercially available as Microcelac^{M}). Preferred are microcrystalline cellulose and mannitol. The total amount of diluent or filler in the pharmaceutical compositions of the present invention may conveniently range from about 20% to about 95% w/w and preferably ranges from about 55% to about 95% w/w, or from about 70% to about 95% w/w, or from about 80% to about 95% w/w, or from about 85% to about 95%.

Lubricants and glidants can be employed in the manufacture of certain dosage forms and will usually be employed when producing tablets. Examples of lubricants and glidants are hydrogenated vegetable oils, e.g hydrogenated Cottonseed oil, magnesium stearate, stearic acid, sodium lauryl sulfate, magnesium lauryl sulfate, colloidal silica, colloidal anhydrous silica talc, mixtures thereof, and others known in the art. Interesting lubricants are magnesium stearate, and mixtures of magnesium stearate with colloidal silica, magnesium stearate being preferred. A preferred glidant is colloidal anhydrous silica.

If present, glidants generally comprise 0.2 to 7.0% w/w of the total composition weight, in particular 0.5 to 1.5% w/w, more in particular 1 to 1.5% w/w.

If present, lubricants generally comprise 0.2 to 7.0% w/w of the total composition weight, in particular 0.2 to 2% w/w, or 0.5 to 2% w/w, or 0.5 to 1.75% w/w, or 0.5 to 1.5% w/w.

Binders can optionally be employed in the pharmaceutical compositions of the present invention. Suitable binders are water-soluble polymers, such as alkylcelluloses such as methylcellulose; hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectines such as sodium carboxymethylamylopectine; chitin derivates such as chitosan; di-, oligo- and polysaccharides such as trehalose, cyclodextrins and derivatives thereof, alginic acid, alkali metal and ammonium salts thereof, carrageenans, galactomannans, tragacanth, agar agar, gummi arabicum, guar gummi and xanthan gummi; polyacrylic acids and the salts thereof; polymethacrylic acids, the salts and esters thereof, methacrylate copolymers; polyvinylpyrrolidone (PVP), polyvinylalcohol (PVA) and copolymers thereof, e.g. PVP-VA. Preferably, the water-soluble polymer is a hydroxyalkyl alkylcelluloses, such as for example hydroxypropylmethyl cellulose, e.g. hydroxypropylmethyl cellulose 15 cps.

Other excipients such as coloring agents and pigments may also be added to the compositions of the invention. Coloring agents and pigments include titanium dioxide and dyes suitable for food. A coloring agent or a pigment is an optional ingredient in the formulation of the invention, but when used the coloring agent can be present in an amount up to 3.5% w/w based on the total composition weight.

Flavors are optional in the composition and may be chosen from synthetic flavor oils and flavoring aromatics or natural oils, extracts from plants leaves, flowers, fruits and so forth and combinations thereof. These may include cinnamon oil, oil of wintergreen, peppermint oils, bay oil, anise oil, eucalyptus, thyme oil. Also useful as flavors are vanilla, citrus oil, including lemon, orange, grape, lime and grapefruit, and fruit essences, including apple, banana, pear, peach, strawberry, raspberry, cherry, plum, pineapple, apricot and so forth. The amount of flavor may depend on a number of factors including the organoleptic effect desired. Generally the flavor will be present in an amount from about 0% to about 3% (w/w).

Formaldehyde scavengers are compounds that are capable of absorbing formaldehyde. They include compounds comprising a nitrogen center that is reactive with formaldehyde, such as to form one or more reversible or irreversible bonds between the formaldehyde scavenger and formaldehyde. For example, the formaldehyde scavenger comprises one or more nitrogen atoms/centers that are reactive with formaldehyde to form a schiff base imine that is capable of subsequently binding with formaldehyde. For example, the formaldehyde scavenger comprises one or more nitrogen centers that are reactive with formaldehyde to form one or more 5-8 membered cyclic rings. The formaldehyde scavenger preferably comprises one or more amine or amide groups. For example, the formaldehyde scavenger can be an amino acid, an amino sugar, an alpha amine compound, or a conjugate or derivative thereof, or a mixture thereof. The formaldehyde scavenger may comprise two or more amines and/or amides.

Formaldehyde scavengers include, for example, glycine, alanine, serine, threonine, cysteine, valine, leucine, isoleucine, methionine, phenylalanine, tyrosine, aspartic acid, glutamic acid, arginine, lysine, ornithine, citrulline, taurine pyrrolysine, meglumine, histidine, aspartame, proline, tryptophan, citrulline, pyrrolysine, asparagine, glutamine, or a conjugate or mixture thereof, or, whenever possible, pharmaceutically acceptable salts thereof.

In an aspect of the invention, the formaldehyde scavenger is meglumine or a pharmaceutically acceptable salt thereof, in particular meglumine base.

In an embodiment, in the methods and uses as described herein, erdafitinib is administered or is to be administered as a pharmaceutical composition, in particular a tablet or capsule, comprising erdafitinib or a pharmaceutically acceptable salt thereof, in particular erdafitinib base; a formaldehyde scavenger, in particular meglumine or a pharmaceutically acceptable salt thereof, in particular meglumine base; and a pharmaceutically acceptable carrier.

It is another object of the invention to provide a process of preparing a pharmaceutical composition as described herein, in particular in the form of a tablet or a capsule, characterized by blending a formaldehyde scavenger, in particular meglumine, and erdafitinib, a pharmaceutically acceptable salt thereof or a solvate thereof, in particular erdafitinib base, with a pharmaceutically acceptable carrier and compressing said blend into tablets or filling said blend in capsules.

Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

It is especially advantageous to formulate the aforementioned pharmaceutical compositions in dosage unit form for ease of administration and uniformity of dosage. Dosage unit form as used herein refers to physically discrete units suitable as unitary dosages, each unit containing a predetermined quantity of active ingredient, calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof. Preferred forms are tablets and capsules.

In certain embodiments, the FGFR inhibitor is present in a solid unit dosage form, and a solid unit dosage form suitable for oral administration. The unit dosage form may contain about 1, 2, 3, 4, 5, 6, 7, 8, 9, or 10 mg of the FGFR inhibitor per unit dose form or an amount in a range bounded by two of these values, in particular 3, 4 or 5 mg per unit dose.

Depending on the mode of administration, the pharmaceutical composition will preferably comprise from 0.05 to 99% by weight, more preferably from 0.1 to 70% by weight, even more preferably from 0.1 to 50% by weight of the FGFR inhibitor, and, from 1 to 99.95% by weight, more preferably from 30 to 99.9% by weight, even more preferably from 50 to 99.9% by weight of a pharmaceutically acceptable carrier, all percentages being based on the total weight of the composition.

Tablets or capsules of the present invention may further be film-coated e.g., to improve taste, to provide ease of swallowing and an elegant appearance. Polymeric film-coating materials are known in the art. Preferred film coatings are water-based film coatings opposed to solvent based film coatings because the latter may contain more traces of aldehydes. A preferred film-coating material is Opadry® II aqueous film coating system, e.g., Opadry® II 85F, such as Opadry® II 85F92209. Further preferred film coatings are water-based film coatings that protects from environmental moisture, such as Readilycoat® (e.g., Readilycoat® D), AquaPolish® MS, Opadry® amb, Opadry® amb II, which are aqueous moisture barrier film coating systems. A preferred film-coating is Opadry® amb II, a high-performance moisture barrier film coating which is a PVA-based immediate release system, without polyethylene glycol.

In tablets according to the invention, the film coat in terms of weight preferably accounts for about 4% (w/w) or less of the total tablet weight.

For capsules according to the present invention, hypromellose (HPMC) capsules are preferred over gelatin capsules.

In an aspect of the invention, the pharmaceutical compositions as described herein, in particular in the form of a capsule or a tablet, comprise from 0.5 mg to 20 mg base equivalent, or from 2 mg to 20 mg base equivalent, or from 0.5 mg to 12 mg base equivalent, or from 2 mg to 12 mg base equivalent, or from 2 mg to 10 mg base equivalent, or from 2 mg to 6 mg base equivalent, or 2 mg base equivalent, 3 mg base equivalent, 4 mg base equivalent, 5 mg base equivalent, 6 mg base equivalent, 7 mg base equivalent, 8 mg base equivalent, 9 mg base equivalent, 10 mg base equivalent, 11 mg base equivalent or 12 mg base equivalent of erdafitinib, a pharmaceutically acceptable salt thereof or a solvate thereof. In particular, the pharmaceutical compositions as described herein comprise 3 mg base equivalent, 4 mg base equivalent or 5 mg base equivalent of erdafitinib, a pharmaceutically acceptable salt thereof or a solvate thereof, in particular 3 mg or 4 mg or 5 mg of erdafitinib base.

In an aspect of the invention, the pharmaceutical compositions as described herein, in particular in the form of a capsule or a tablet, comprise from 0.5 mg to 20 mg, or from 2 mg to 20 mg, or from 0.5 mg to 12 mg, or from 2 mg to 12 mg, or from 2 mg to 10 mg, or from 2 mg to 6 mg, or 2 mg, 3 mg, 4 mg, 5 mg, 6 mg, 7 mg, 8 mg, 9 mg, 10 mg, 11 mg or 12 mg of erdafitinib base. In particular, the pharmaceutical compositions as described herein comprise 3 mg, 4 mg or 5 mg of erdafitinib base. In particular, the pharmaceutical compositions as described herein comprise 3 mg, 4 mg or 5 mg of erdafitinib base and from about 0.5 to about 5% w/w, from about 0.5 to about 3% w/w, from about 0.5 to about 2% w/w, from about 0.5 to about 1.5% w/w, or from about 0.5 to about 1% w/w of a formaldehyde scavenger, in particular meglumine. In particular, the pharmaceutical compositions as described herein comprise 3 mg, 4 mg or 5 mg of erdafitinib base and from about 0.5 to about 1.5% w/w or from about 0.5 to about 1% w/w of a formaldehyde scavenger, in particular meglumine.

In an aspect of the invention, more than one, e.g., two, pharmaceutical compositions as described herein can be administered in order to obtain a desired dose, e.g., a daily dose. For example, for a daily dose of 8 mg base equivalent of erdafitinib, 2 tablets or capsules of 4 mg erdafitinib base equivalent each may be administered; or a tablet or a capsule of 3 mg erdafitinib base equivalent and a tablet or capsule of 5 mg base equivalent may be administered. For example, for a daily dose of 9 mg base equivalent of erdafitinib, 3 tablets or capsules of 3 mg erdafitinib base equivalent each may be administered; or a tablet or a capsule of 4 mg erdafitinib base equivalent and a tablet or capsule of 5 mg base equivalent may be administered.

The amount of formaldehyde scavenger, in particular meglumine, in the pharmaceutical compositions according to the present invention may range from about 0.1 to about 10% w/w, about 0.1 to about 5% w/w, from about 0.1 to about 3% w/w, from about 0.1 to about 2% w/w, from about 0.1 to about 1.5% w/w, from about 0.1 to about 1% w/w, from about 0.5 to about 5% w/w, from about 0.5 to about 3% w/w, from about 0.5 to about 2% w/w, from about 0.5 to about 1.5% w/w, from about 0.5 to about 1% w/w.

According to particular embodiments, erdafitinib is supplied as 3 mg, 4 mg or 5 mg film-coated tablets for oral administration and contains the following inactive ingredients or equivalents thereof: Tablet Core: croscarmellose sodium, magnesium stearate, mannitol, meglumine, and microcrystalline cellulose; and Film Coating: Opadry amb II: Glycerol monocaprylocaprate Type I, polyvinyl alcohol-partially hydrolyzed, sodium lauryl sulfate, talc, titanium dioxide, iron oxide yellow, iron oxide red (for orange and brown tablets), ferrosoferric oxide/iron oxide black (for brown tablets).

Studies that look at safety seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “acceptable” with respect to a formulation, composition or ingredient, as used herein, means that the beneficial effects of that formulation, composition or ingredient on the general health of the human being treated substantially outweigh its detrimental effects, to the extent any exist.

All formulations for oral administration are in dosage form suitable for such administration.

### Methods of Dosing and Treatment Regimens

In one aspect, described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2. In one aspect, described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR fusion selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1. Also described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, or parathyroid carcinoma. Also described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma.

Also described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR genetic alteration, wherein the cancer is cholangiocarcinoma, high-grade glioma, pancreatic cancer, squamous NSCLC, non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, gastrointestinal stromal tumor, or parathyroid carcinoma. Also described herein are methods of treating cancer, said methods comprising, consisting of, or consisting essentially of, administering a therapeutically effective amount of an FGFR inhibitor generally, and erdafitinib specifically, to a patient who has been diagnosed with cancer and who harbors at least one FGFR genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancers, esophageal cancer, low-grade glioma, prostate cancer, salivary gland cancer, basal cell carcinoma, thymic cancer, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma.

In some embodiments, the FGFR inhibitor generally, and erdafitinib specifically is administered daily, in particular once daily. In some embodiments, the FGFR inhibitor generally, and erdafitinib specifically is administered twice-a-day. In some embodiments, the FGFR inhibitor generally, and erdafitinib specifically is administered three times a day. In some embodiments, the FGFR inhibitor generally, and erdafitinib specifically is administered four times a day. In some embodiments, the FGFR inhibitor generally, and erdafitinib specifically is administered every other day. In some embodiments, the FGFR inhibitor generally, and erdafitinib specifically is administered weekly. In some embodiments, the FGFR inhibitor generally, and erdafitinib specifically is administered twice a week. In some embodiments, the FGFR inhibitor generally, and erdafitinib specifically is administered every other week. In some embodiments, the FGFR inhibitor generally, and erdafitinib specifically is administered orally on a continuous daily dosage schedule.

In general, doses of the FGFR inhibitor, and erdafitinib specifically, employed for treatment of the diseases or conditions described herein in humans are typically in the range of about 1 to 20 mg per day. In some embodiments, the FGFR inhibitor, and erdafitinib specifically, is administered orally to the human at a dose of about 1 mg per day, about 2 mg per day, about 3 mg per day, about 4 mg per day, about 5 mg per day, about 6 mg per day, about 7 mg per day, about 8 mg per day, about 9 mg per day, about 10 mg per day, about 11 mg per day, about 12 mg per day, about 13 mg per day, about 14 mg per day, about 15 mg per day, about 16 mg per day, about 17 mg per day, about 18 mg per day, about 19 mg per day or about 20 mg per day.

Also described herein are methods of treating cancer in a cancer patient, comprising administering erdafitinib to the cancer patient, wherein the age of the cancer patient ranges from and including 15 years to <18 years. Also described herein is the use of erdafitinib for the manufacture of a medicament for the treatment of cancer in a cancer patient, wherein the age of the cancer patient ranges from and including 15 years to <18 years. Also described herein is erdafitinib for use in the treatment of cancer in a cancer patient, wherein the age of the cancer patient ranges from and including 15 years to <18 years.

In some embodiments, erdafitinib is administered orally. In certain embodiments, if the patient is 15 years of age or older at the date of first administration of said FGFR inhibitor, in particular erdafitinib, the FGFR inhibitor, in particular erdafitinib, is administered orally at a dose of about 8 mg once daily. In further embodiments, the dose of erdafitinib is increased from 8 mg once daily to 9 mg once daily. In still further embodiments, the dose of erdafitinib is increased from 8 mg once daily to 9 mg once daily at 14 days after initiating treatment if the patient exhibits a serum phosphate (PO₄) level that is less than about 7.0 mg/dL at 14 days, optionally 14 plus 2 days, in particular at 14 days, after initiating treatment. In further embodiments, the dose of erdafitinib is increased from 8 mg per day to 9 mg per day after initiating treatment if the patient exhibits a serum PO₄ level that ranges from and including 7.0 mg/dL to <9.0 mg/dL, in particular the dose of erdafitinib is increased from 8 mg per day to 9 mg per day after initiating treatment if the patient exhibits a serum PO₄ level that ranges from and including 7.0 mg/dL to <9.0 mg/dL at 14 days, optionally 14 plus 2 days, in particular at 14 days, after initiating treatment. In certain embodiments, the increase in dose of erdafitinib from 8 mg to 9 mg is in combination with administration of a phosphate binder, e.g., sevelamer. In certain embodiments, the increase in dose of erdafitinib from 8 mg to 9 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment. In an embodiment the patient is an adult at the date of first administration of said FGFR inhibitor, in particular erdafitinib. In an embodiment the patient is 18 years of age or older at the date of first administration of said FGFR inhibitor, in particular erdafitinib. In an embodiment the patient is an adolescent at the date of first administration of said FGFR inhibitor, in particular erdafitinib. In an embodiment the patient is aged ranging from and including 15 years to <18 years at the date of first administration of said FGFR inhibitor, in particular erdafitinib.

In an embodiment, erdafitinib is administered at a dose of 8 mg, in particular 8 mg once daily. In an embodiment, erdafitinib is administered at a dose of 8 mg, in particular 8 mg once daily, with an option to uptitrate to 9 mg depending on serum phosphate levels (e.g. serum phosphate levels are <7 mg/dL or range from and include 7 mg/dL to <9 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of erdafitinib administration. In an embodiment the patient is an adult at the date of first administration of said FGFR inhibitor, in particular erdafitinib. In an embodiment the patient is 18 years of age or older at the date of first administration of said FGFR inhibitor, in particular erdafitinib. In an embodiment the patient is an adolescent at the date of first administration of said FGFR inhibitor, in particular erdafitinib. In an embodiment the patient is aged ranging from and including 15 years to <18 years at the date of first administration of said FGFR inhibitor, in particular erdafitinib. In an embodiment the patient is 15 years of age or older at the date of first administration of said FGFR inhibitor, in particular erdafitinib.

In some embodiments, if the patient is between 12 years of age and <15 years of age at the date of first administration of said FGFR inhibitor, in particular erdafitinib, the FGFR inhibitor, in particular erdafitinib, is administered at a dose of about 5 mg, in particular 5 mg once daily. As used herein, “between” is inclusive of the lower age range. For example, between 12 years of age and <15 years of age includes patients who are 12 years of age. Also as used herein, the upper age range includes patients up to the day before the patient turns the indicated age, e.g. 15 years of age. In an embodiment, erdafitinib is administered at a dose of 5 mg, in particular 5 mg once daily, with an option to uptitrate to 6 mg, and with a further option to uptitrate from 6 mg to 8 mg, depending on serum phosphate levels (e.g. serum phosphate levels are <7 mg/dL or range from and include 7 mg/dL to <9 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of the first cycle of erdafitinib administration. As used herein, day 14 after initiating treatment, day 14 of the first cycle of erdafitinib administration, Cycle 1 Day 14 and C1D14 are used interchangeably. In an embodiment, the levels of serum phosphate for determining whether or not to up-titrate are measured on a treatment day during the second cycle of erdafitinib treatment, in particular on day 7 of the second cycle of erdafitinib administration (Cycle 2 Day 7 or C2D7). In certain embodiments, erdafitinib is administered at a dose of about 5 mg once daily. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at 7 or 14 days, optionally 14 plus 2 days, in particular at 14 days, after initiating treatment. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at day 7 of the second cycle of erdafitinib treatment. In certain embodiments, the dose of erdafitinib is further increased from 6 mg per day to 8 mg per day after initiating treatment if the patient exhibits a serum PO₄ level that ranges from and includes 7.0 to <9 mg/dL at 14 days, optionally 14 plus 2 days, in particular at 14 days, after initiating treatment. In certain embodiments, the dose of erdafitinib is further increased from 6 mg per day to 8 mg per day after initiating treatment if the patient exhibits a serum PO₄ level that ranges from and includes 7.0 to <9 mg/dL at day 7 of the second cycle of erdafitinib treatment. In certain embodiments, the increase in dose of erdafitinib from 5 mg to 6 mg or from 6 mg to 8 mg is in combination with administration of a phosphate binder, e.g. sevelamer. In certain embodiments, the increase in dose of erdafitinib from 5 mg to 6 mg or from 6 mg to 8 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment or at day 7 of the second cycle of erdafitinib treatment. In still further embodiments, the dose of erdafitinib is increased from 5 mg per day to 6 mg per day after initiating treatment if the patient exhibits a serum P04 level of less than 7.0 mg/dL at 7 or 14 days, optionally 14 plus 2 days, in particular at 14 days, after initiating treatment. In still further embodiments, the dose of erdafitinib is increased from 5 mg per day to 6 mg per day after initiating treatment if the patient exhibits a serum P04 level of less than 7.0 mg/dL at 14 days, optionally 14 plus 2 days, in particular at 14 days, after initiating treatment. In still further embodiments, the dose of erdafitinib is increased from 5 mg per day to 6 mg per day after initiating treatment if the patient exhibits a serum PG4 level of less than 7.0 mg/dL at day 7 of the second cycle of erdafitinib treatment. In further embodiments, the dose of erdafitinib is further increased from 6 mg per day to 8 mg per day after initiating treatment if the patient exhibits a serum PO₄ level of less than 7.0 mg/dL at 14 days, optionally 14 plus 2 days, in particular at 14 days, after initiating treatment. In further embodiments, the dose of erdafitinib is further increased from 6 mg per day to 8 mg per day after initiating treatment if the patient exhibits a serum PO₄ level of less than 7.0 mg/dL at day 7 of the second cycle of erdafitinib treatment. In further embodiments, the 2-step up-titration (from 5 mg to 6 mg, and from 6 mg to 8 mg) is stepwise, i.e., no subject is allowed to directly up-titrate from 5 mg to 8 mg.

In some embodiments, if the patient is between 6 years of age and <12 years of age at the date of first administration of said FGFR inhibitor, in particular erdafitinib, the FGFR inhibitor, in particular erdafitinib, is administered at a dose of about 3 mg, in particular 3 mg once daily. As used herein, “between” is inclusive of the lower age range. For example, between 6 years of age and <12 years of age includes patients who are 6 years of age. Also as used herein, the upper age range includes patients up to the day before the patient turns the indicated age, e.g. 12 years of age. In an embodiment, erdafitinib is administered at a dose of 3 mg, in particular 3 mg once daily, with an option to up-titrate to 4 mg, and with a further option to up-titrate from 4 mg to 5 mg, depending on serum phosphate levels (e.g. serum phosphate levels are <7 mg/dL or range from and include 7 mg/dL to <9 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of the first cycle of erdafitinib administration. As used herein, day 14 after initiating treatment, day 14 of the first cycle of erdafitinib administration, Cycle 1 Day 14 and C1D14 are used interchangeably. In an embodiment, the levels of serum phosphate for determining whether or not to up-titrate are measured on a treatment day during the second cycle of erdafitinib treatment, in particular on day 7 of the second cycle of erdafitinib administration (Cycle 2 Day 7 or C2D7). In certain embodiments, erdafitinib is administered at a dose of about 3 mg once daily. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at 14 days, optionally 14 plus 2 days, after initiating treatment. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at day 7 of the second cycle of erdafitinib treatment. In certain embodiments, the dose of erdafitinib is further increased from 4 mg per day to 5 mg per day after initiating treatment if the patient exhibits a serum PO₄ level that ranges from and includes 7.0 to <9 mg/dL at day 7 of the second cycle of erdafitinib treatment. In certain embodiments, the increase in dose of erdafitinib from 3 mg to 4 mg or from 4 mg to 5 mg is in combination with administration of a phosphate binder, e.g. sevelamer. In certain embodiments, the increase in dose of erdafitinib from 3 mg to 4 mg or from 4 mg to 5 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment or at day 7 of the second cycle of erdafitinib treatment. In still further embodiments, the dose of erdafitinib is increased from 3 mg per day to 4 mg per day after initiating treatment if the patient exhibits a serum PO₄ level of less than 7.0 mg/dL at 14 days, optionally 14 plus 2 days, in particular at 14 days, after initiating treatment. In still further embodiments, the dose of erdafitinib is increased from 3 mg per day to 4 mg per day after initiating treatment if the patient exhibits a serum PO₄ level of less than 7.0 mg/dL at day 7 of the second cycle of erdafitinib treatment. In further embodiments, the dose of erdafitinib is further increased from 4 mg per day to 5 mg per day after initiating treatment if the patient exhibits a serum PO₄ level of less than 7.0 mg/dL at day 7 of the second cycle of erdafitinib treatment. In further embodiments, the 2-step up-titration (from 3 mg to 4 mg, and from 4 mg to 5 mg) is stepwise, i.e., no subject is allowed to directly up-titrate from 3 mg to 5 mg.

For all embodiments described herein, “between” is inclusive of the lower age range. For example, between 12 years of age and <15 years of age includes patients who are 12 years of age. Additionally, between 6 years of age and <12 years of age includes patients who are 6 years of age

For all embodiments described herein, the upper age range includes patients up to the day before the patient turns the indicated age. For example, between 12 years of age and <15 years of age includes patients up until the day before they turn 15 years of age. Additionally, between 6 years of age and <12 years of age includes patients up until the day before they turn 12 years of age.

Also described herein are methods of treating cancer in a cancer patient, comprising administering erdafitinib to the cancer patient, wherein the age of the cancer patient ranges from and including 12 years to <15 years. Also described herein is the use of erdafitinib for the manufacture of a medicament for the treatment of cancer in a cancer patient, wherein the age of the cancer patient ranges from and including 12 years to <15 years. Also described herein is erdafitinib for use in the treatment of cancer in a cancer patient, wherein the age of the cancer patient ranges from and including 12 years to <15 years.

In some embodiments, if the patient is between 12 years of age and <15 years of age at the date of first administration of said FGFR inhibitor, in particular erdafitinib, the FGFR inhibitor, in particular erdafitinib, is administered at a dose of about 5 mg, in particular 5 mg once daily. In an embodiment, erdafitinib is administered at a dose of 5 mg, in particular 5 mg once daily, with an option to uptitrate to 6 mg, and with a further option to uptitrate from 6 mg to 8 mg, depending on serum phosphate levels (e.g. serum phosphate levels are <7 mg/dL or range from and include 7 mg/dL to <9 mg/dL), and depending on treatment-related adverse events observed. In certain embodiments, erdafitinib is administered at a dose of about 5 mg once daily. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at 14 days, optionally 14 plus 2 days, in particular at 14 days, after initiating treatment. In further embodiments, the dose of erdafitinib is increased from 5 mg per day to 6 mg per day after initiating treatment if the patient exhibits a serum phosphate (PO₄) level that ranges from and includes 7.0 mg/dL to <9 mg/dL at day 7 of the second cycle of erdafitinib treatment. In certain embodiments, the dose of erdafitinib is increased from 6 mg per day to 8 mg per day after initiating treatment if the patient exhibits a serum PO₄ level that ranges from and includes 7.0 to <9 mg/dL at day 7 of the second cycle of erdafitinib treatment for those patients already up-titrated from 5 mg to 6 mg at 14 days, optionally 14 plus 2 days, in particular at 14 days, after initiating treatment. In certain embodiments, the increase in dose of erdafitinib from 5 mg to 6 mg or from 6 mg to 8 mg is in combination with administration of a phosphate binder, e.g. sevelamer. In certain embodiments, the increase in dose of erdafitinib from 5 mg to 6 mg or from 6 mg to 8 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment or at day 7 of the second cycle of erdafitinib treatment. In still further embodiments, the dose of erdafitinib is increased from 5 mg per day to 6 mg per day after initiating treatment if the patient exhibits a serum P04 level of less than 7.0 mg/dL at 14 days, optionally 14 plus 2 days, in particular at 14 days, after initiating treatment. In still further embodiments, the dose of erdafitinib is increased from 5 mg per day to 6 mg per day after initiating treatment if the patient exhibits a serum PO₄ level of less than 7.0 mg/dL at day 7 of the second cycle of erdafitinib treatment. In further embodiments, the dose of erdafitinib is increased from 6 mg per day to 8 mg per day after initiating treatment if the patient exhibits a serum PO₄ level of less than 7.0 mg/dL at day 7 of the second cycle of erdafitinib treatment for those patients already up-titrated from 5 mg to 6 mg at 14 days, optionally 14 plus 2 days, in particular at 14 days, after initiating treatment.

Also described herein are methods of treating cancer in a cancer patient, comprising administering erdafitinib to the cancer patient, wherein the age of the cancer patient ranges from and including 6 years to <12 years. Also described herein is the use of erdafitinib for the manufacture of a medicament for the treatment of cancer in a cancer patient, wherein the age of the cancer patient ranges from and including 6 years to <12 years. Also described herein is erdafitinib for use in the treatment of cancer in a cancer patient, wherein the age of the cancer patient ranges from and including 6 years to <12 years.

In some embodiments, if the patient is between 6 years of age and <12 years of age at the date of first administration of said FGFR inhibitor, in particular erdafitinib, the FGFR inhibitor, in particular erdafitinib, is administered at a dose of about 3 mg, in particular 3 mg once daily. In an embodiment, erdafitinib is administered at a dose of 3 mg, in particular 3 mg once daily, with an option to up-titrate to 4 mg, and with a further option to up-titrate from 4 mg to 5 mg, depending on serum phosphate levels (e.g. serum phosphate levels are <7 mg/dL or range from and include 7 mg/dL to <9 mg/dL), and depending on treatment-related adverse events observed. In an embodiment, the levels of serum phosphate for determining whether or not to up-titrate are measured on a treatment day during the first cycle of erdafitinib treatment, in particular on day 14 plus 2 days, more in particular on day 14, of the first cycle of erdafitinib administration. As used herein, day 14 after initiating treatment, day 14 of the first cycle of erdafitinib administration, Cycle 1 Day 14 and C1D14 are used interchangeably. In an embodiment, the levels of serum phosphate for determining whether or not to up-titrate are measured on a treatment day during the second cycle of erdafitinib treatment, in particular on day 7 of the second cycle of erdafitinib administration (Cycle 2 Day 7 or C2D7). In certain embodiments, erdafitinib is administered at a dose of about 3 mg once daily. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at 14 days, optionally 14 plus 2 days, after initiating treatment. In further embodiments, the dose of erdafitinib is increased from 3 mg per day to 4 mg per day after initiating treatment if the patient exhibits a serum phosphate (PO₄) level that ranges from and includes 7.0 mg/dL to <9 mg/dL at day 7 of the second cycle of erdafitinib treatment. In certain embodiments, the dose of erdafitinib is increased from 4 mg per day to 5 mg per day after initiating treatment if the patient exhibits a serum PO₄ level that ranges from and includes 7.0 to <9 mg/dL at day 7 of the second cycle of erdafitinib treatment for those patients already up-titrated from 3 mg to 4 mg at 14 days, optionally 14 plus 2 days, in particular at 14 days, after initiating treatment. In certain embodiments, the increase in dose of erdafitinib from 3 mg to 4 mg or from 4 mg to 5 mg is in combination with administration of a phosphate binder, e.g. sevelamer. In certain embodiments, the increase in dose of erdafitinib from 3 mg to 4 mg or from 4 mg to 5 mg is in combination with administration of a phosphate binder, e.g. sevelamer, if the serum PO₄ level ranges from and including 7.0 mg/dL to <9.0 mg/dL at 14 days, optionally 14 plus 2 days, in particular at 14 days, after initiating treatment or at day 7 of the second cycle of erdafitinib treatment. In still further embodiments, the dose of erdafitinib is increased from 3 mg per day to 4 mg per day after initiating treatment if the patient exhibits a serum PO₄ level of less than 7.0 mg/dL at 14 days, optionally 14 plus 2 days, in particular at 14 days, after initiating treatment. In still further embodiments, the dose of erdafitinib is increased from 3 mg per day to 4 mg per day after initiating treatment if the patient exhibits a serum PO₄ level of less than 7.0 mg/dL at day 7 of the second cycle of erdafitinib treatment. In further embodiments, the dose of erdafitinib is increased from 4 mg per day to 5 mg per day after initiating treatment if the patient exhibits a serum PO₄ level of less than 7.0 mg/dL at day 7 of the second cycle of erdafitinib treatment for those patients already up-titrated from 3 mg to 4 mg at 14 days, optionally 14 plus 2 days, in particular at 14 days, after initiating treatment.

In an embodiment, the treatment cycle as used herein is a 28-day cycle. In certain embodiments, the treatment cycle is a 28-day cycle for up to two years.

In a preferred embodiment, the treatment cycle as used herein is a 21-day cycle. In particular, the treatment is a continuous 21-day cycle treatment.

In one embodiment, the desired dose is conveniently presented in a single dose or in divided doses administered simultaneously (or over a short period of time) or at appropriate intervals, for example as two, three, four or more sub-doses per day. In some embodiments, the FGFR inhibitor is conveniently presented in divided doses that are administered simultaneously (or over a short period of time) once a day. In some embodiments, the FGFR inhibitor generally, and erdafitinib specifically, is conveniently presented in divided doses that are administered in equal portions twice-a-day. In some embodiments, the FGFR inhibitor generally, and erdafitinib specifically, is conveniently presented in divided doses that are administered in equal portions three times a day. In some embodiments, the FGFR inhibitor is conveniently presented in divided doses that are administered in equal portions four times a day.

In certain embodiments, the desired dose may be delivered in 1, 2, 3, 4, 5, 6, 7, 8, 9, or 10 fractional unit dosages throughout the course of the day, such that the total amount of FGFR inhibitor generally, and erdafitinib specifically, delivered by the fractional unit dosages over the course of the day provides the total daily dosages.

In some embodiments, the amount of the FGFR inhibitor generally, and erdafitinib specifically, that is given to the human varies depending upon factors such as, but not limited to, condition and severity of the disease or condition, and the identity (e.g., weight) of the human, and the particular additional therapeutic agents that are administered (if applicable).

In still further embodiments, erdafitinib is not co-administered with strong CYP3A4 inhibitors or inducers or moderate CyP3A4 inducers. In certain embodiments, erdafitinib is not co-administered with strong CYP3A4 inhibitors or inducers or moderate CyP3A4 inducers within 14 days or 5 half-lives before the first dose of study drug.

Non-limiting examples of strong CYP3A4 inhibitors include Boceprevir, Aprepitant, Clarithromycin, Conivaptan, grapefruit juice, Indinavir, Lopinavir Itraconazole, Mibefradil Ketoconazole, Nefazodone, Ritonavir, Posaconazole, Nelfinavir, Saquinavir, Conivaptan, Telaprevir, Boceprevir, Telithromycin, Clarithromycin, Voriconazole, Clotrimazole, Diltiazem, Erythromycin, Fluconazole, Verapamil, and Troleandomycin.

Non-limiting examples of moderate to strong CYP3A4 inducers include Avasimibe, St. John's wort, Carbamazepine, Efavirenz, Phenytoin, Etravirine, Bosentan, Nafcillin, Rifampin, Modafinil, Rifabutin, and Barbiturates.

### Kits/Articles of Manufacture

For use in the method or uses described herein, kits and articles of manufacture are also described. Such kits include a package or container that is compartmentalized to receive one or more dosages of the pharmaceutical compositions disclosed herein. Suitable containers include, for example, bottles. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert.

In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The examples and embodiments described herein are for illustrative purposes only and various modifications or changes suggested to persons skilled in the art are to be included within the spirit and purview of this application and scope of the appended claims.

### Example 1: Identification of FGFR Mutations with Oncogenic Driver Potential

A) In certain embodiments, target FGFR mutations include: FGFR1-K656E; FGFR1-R189C; FGFR1-S125L; FGFR1-P150S; FGFR2-C390Y; FGFR2-E565G; FGFR2-E565Q; FGFR2-S252L; FGFR2-C382F; FGFR2-P253L; FGFR2-A97T; FGFR2-R251Q; FGFR2-A389T; FGFR2-S252P; FGFR2-R210Q; FGFR2-S252T; FGFR2-R203H; FGFR2-S252A; FGFR2-S351C; FGFR2-Y340C; FGFR2-G338R; FGFR2-S354C; FGFR2-L617F; FGFR2-W290R; FGFR2-L550F; FGFR2-M5351; FGFR2-Y308C; FGFR2-E777*; FGFR2-K641R; FGFR2-T370R; FGFR2-W72C; FGFR2-K526E; FGFR2-D304N; FGFR2-K659M; FGFR2-S267P; FGFR2-E731K; FGFR2-M5371; FGFR2-F276C; FGFR2-1547V; FGFR2-E565A; FGFR2-V395D; FGFR2-W290C; FGFR2-R678G; FGFR2-E777K; FGFR2-C382R; FGFR2-S372C; FGFR2-A315T; FGFR2-D101Y; FGFR2-Y375C; FGFR2-E219K; FGFR2-L770*; FGFR2-L770V; FGFR2-K659N; FGFR3-M5281; FGFR3-K650T; FGFR3-S371G; FGFR3-K650N; FGFR3-G380E; FGFR3-E627D; FGFR3-R399C; FGFR3-Y373N; FGFR3-Y373H; FGFR3-A500T; FGFR3-D641N; FGFR3-S249Y; FGFR3-A391V; FGFR3-S249F; FGFR3-S371R; FGFR3-R248H; FGFR3-G370S; FGFR3-P572A; FGFR3-P572L; FGFR3-R669Q; FGFR3-P250R; FGFR3-Y278C; FGFR3-L324V; FGFR3-S84L; FGFR3-R750C; FGFR3-S433C; FGFR3-K650Q; FGFR3-S371C; FGFR3-S249C; FGFR3-F384L; FGFR3-G370C; FGFR3-R248C; FGFR3-Y373C; and FGFR4-Y367C. Target FGFR mutations were subjected to preclinical evaluation for sensitivity to erdafitinib.
B) In certain embodiments, target FGFR mutations include: FGFR1-K656E; FGFR1-R189C; FGFR1-S125L; FGFR1-P150S; FGFR2-C390Y; FGFR2-E565G; FGFR2-E565Q; FGFR2-S252L; FGFR2-C382F; FGFR2-P253L; FGFR2-R251Q; FGFR2-A389T; FGFR2-S252P; FGFR2-R210Q; FGFR2-S252T; FGFR2-R203H; FGFR2-S252A; FGFR2-S351C; FGFR2-Y340C; FGFR2-G338R; FGFR2-S354C; FGFR2-L617F; FGFR2-W290R; FGFR2-L550F; FGFR2-M5351; FGFR2-Y308C; FGFR2-E777*; FGFR2-K641R; FGFR2-T370R; FGFR2-W72C; FGFR2-K526E; FGFR2-D304N; FGFR2-K659M; FGFR2-S267P; FGFR2-E731K; FGFR2-M5371; FGFR2-F276C; FGFR2-1547V; FGFR2-E565A; FGFR2-V395D; FGFR2-W290C; FGFR2-R678G; FGFR2-E777K; FGFR2-C382R; FGFR2-S372C; FGFR2-A315T; FGFR2-D101Y; FGFR2-Y375C; FGFR2-E219K; FGFR2-L770*; FGFR2-L770V; FGFR2-K659N; FGFR3-M5281; FGFR3-K650T; FGFR3-S371G; FGFR3-K650N; FGFR3-G380E; FGFR3-E627D; FGFR3-Y373N; FGFR3-Y373H; FGFR3-D641N; FGFR3-S249Y; FGFR3-A391V; FGFR3-S249F; FGFR3-S371R; FGFR3-R248H; FGFR3-G370S; FGFR3-R669Q; FGFR3-P250R; FGFR3-Y278C; FGFR3-L324V; FGFR3-S84L; FGFR3-R750C; FGFR3-S433C; FGFR3-K650Q; FGFR3-S371C; FGFR3-S249C; FGFR3-G370C; FGFR3-R248C; FGFR3-Y373C; and FGFR4-Y367C.

FGFR gene alterations, specifically mutations and fusions, may function as oncogenic drivers of disease independent of the underlying tumor histology.

FGFR alterations were found across solid tumor types at varying frequencies (1%-29%) (Table 2) (analysis of data from TCGA (The Cancer Genome Atlas) and GENIE (the AACR Project Genomics Evidence Neoplasia Information Exchange) genomic databases.

TABLE 2
Frequency of FGFR Mutations and Fusions in Advanced Cancer

| Advanced Cancer | | | Predominant alt type |
|---|---|---|---|
| Type | Total ^{a} | Target ^{b} | (mutation vs fusion) ^{c} |
| Urothelial cancer | ~34% | ~29% | Mutations |
| High-grade glioma | ~26% | ~21% | Fusions |
| (Glioblastoma) | | | |
| Squamous cell head and | ~15% | ~9% | Mutations and fusions |
| neck cancers | | | |
| Soft tissue sarcoma | ~10% | ~8% | Fusions |
| Cholangiocarcinoma | | | |
| Intrahepatic | ~11% | ~7% | Fusions |
| Extrahepatic | ~7% | ~4% | Fusions |
| Endometrial | ~15% | ~7% | Mutations |
| Low-grade glioma | ~10% | ~5% | Mutations and fusions |
| Squamous NSCLC | ~9% | ~4% | Mutations and fusions |
| Cervical cancer | ~9% | ~3% | Mutations and fusions |
| Gastric cancer | ~8% | ~3% | Fusions |
| Breast cancer | ~5% | ~2% | Mutations and fusions |
| Ovarian cancer | ~6% | ~2% | Mutations and fusions |
| Hepatocellular | ~5% | ~2% | Fusions |
| carcinoma | | | |
| Renal cell cancer | ~5% | ~1% | Mutations and fusions |
| Esophageal cancer | ~6% | ~1% | Fusions |
| Pancreatic cancer | ~2% | <1% ^{e} | Fusions |
| Salivary gland tumors | ~5% | ~3% | Fusions |
| Colorectal cancer | ~8% | ~1% | Mutations |
| Thymic cancer/thymoma | ~2% | ~1% | Mutations |

NSCLC, non-small-cell lung cancer
^{a} Total refers to all FGFR mutations and fusions identified.
^{b} Target refers to all FGFR mutations and fusions that are potentially pathogenic based on genomic features.
^{c} Mutation vs fusion refers to the target. “Mutations and fusions” denoted in cases where alteration type encompassed ≥⅓ of the alt-positive population.
^{d} Based on clinical experience.

### Example 2: A Phase 2 Study of Erdafitinib in Subjects with Advanced Solid Tumors and FGFR Gene Alterations (NCT04083976)

An ongoing, non-limiting example of a single-arm, open-label, Phase 2 histology-agnostic trial investigating the efficacy and safety of erdafitinib, a selective pan-FGFR inhibitor, in patients with advanced solid tumors and FGFR alterations after failure of standard systemic therapies, is provided herein.

### Overall Study Design

This Phase 2, open-label study (also known as the RAGNAR study) investigates the efficacy and safety of erdafitinib in subjects ≥6 years of age with advanced solid tumors (other than urothelial tumors) and FGFR gene alterations. Subjects ≥12 years of age with target FGFR mutations or any FGFR gene fusions were enrolled into the Broad Panel Cohort. Target FGFR mutations include select mutations based on likelihood for pathogenicity and with preclinical sensitivity to erdafitinib, or those with clinical or correlative evidence supporting inclusion. A subgroup of subjects in the Broad Panel Cohort with a select panel of pre-specified FGFR markers were identified as the Core Panel Cohort (for analysis only). While the Broad Panel Cohort consists of target FGFR mutations and any fusions, the Core Panel Cohort consists of a select subset of FGFR mutations or fusions. Subjects with any other FGFR mutations that are not captured in the Broad Panel Cohort are included in the study as the Exploratory Cohort. A separate Cholangiocarcinoma Expansion Cohort enrolled subjects with target FGFR mutations or any FGFR gene fusion once the Broad Panel Cohort reached the cap of approximately 30 subjects for cholangiocarcinoma. A Pediatric Cohort enrolled all subjects 6 to <18 years of age with locally advanced or metastatic solid tumors harboring FGFR alterations who have either progressed following prior therapies and who have no acceptable standard therapies, or who have a newly-diagnosed solid tumor and who have no acceptable standard therapies. Adolescent subjects enrolled in the Broad Panel Cohort (≥12 to <18 years) are considered part of the Broad Panel Cohort and the Pediatric Cohort.

The Screening Phase starts with the Molecular Eligibility Screening Period. Subjects with study-eligible FGFR alterations may be identified by central next-generation sequencing (NGS) from tissue sample, or based on locally performed and commercial testing from tissue or blood (NGS tests, direct digital counting methods, or the Qiagen Therascreen® FGFR reverse transcription polymerase chain reaction [RT-PCR] test).

Subjects from all solid tumor histologies (except bladder) with an eligible FGFR mutation or fusion identified via local testing results are considered molecularly eligible for the study.

Subjects with advanced solid tumors may receive central molecular screening if they have received at least 1 line of systemic therapy and are anticipated to fulfill study eligibility criteria within 6 months. Central molecular screening is selective to the following tumor histologies: high-grade gliomas (e.g., glioblastoma) and low-grade gliomas; squamous cell head and neck cancers; soft tissue sarcoma; cholangiocarcinoma; endometrial, cervical, and ovarian cancers; squamous NSCLC; renal cell cancer; esophageal and gastric cancers; hormone-sensitive breast cancer (estrogen positive [ER]/progesterone positive [PR]); hepatocellular carcinoma, pancreatic cancer, salivary gland tumors, colorectal cancer, and thymic cancer/thymoma. Central screening for any tumor type will be allowed for pediatric subjects or if a local report is deemed insufficient. For enrollment in each tumor histology, the sample size is capped at approximately 30 subjects. The tumor histology list for this cap, including a group of Other, is pre-defined in Table 4.

TABLE 4
List of Tumor Histologies for Futility and Cap

| Number | Tumor Histologies |
|---|---|
| 1 | Cholangiocarcinoma |
| 2 | High-grade Glioma |
| 3 | Squamous NSCLC |
| 4 | Squamous cell head and neck cancers |
| 5 | Gastric Cancer |
| 6 | Breast Cancer |
| 7 | Endometrial Cancer |
| 8 | Low-grade Glioma |
| 9 | Ovarian Cancer |
| 10 | Non-squamous NSCLC |
| 11 | Colorectal Cancer |
| 12 | Pancreatic Cancer |
| 13 | Cervical Cancer |
| 14 | Esophageal |
| 15 | Other |

Note:
The group Other enrolls on all other tumor histologies not listed. The group “other” shares the same cap of approximately 30 and will be included in the BHM evaluation for information borrowing only but will not be deemed futile early in the interim analyses and will continue enrollment until cap is reached.

The Full-study Screening Period occurs after the completion of prior treatment and documentation of disease progression for subjects who meet the molecular screening criteria.

The Treatment Phase continues until disease progression, intolerable toxicity, withdrawal of consent, or decision by the investigator to discontinue treatment. The post treatment Follow-up Phase extends from the End of Treatment Visit until the subject has died, withdraws consent, is lost to follow-up, or the end of study, whichever comes first.

At the end of the study, subjects who have completed the study and are benefiting from the study intervention, as determined by the investigator, will be discontinued from the trial and should continue treatment with commercially available medication (as part of standard of care), if it is accessible to the subject as applicable per local regulations. If commercial medication is not accessible, subjects who have completed the study and are benefiting from the study intervention, as determined by the investigator, will be able to receive continued access to erdafitinib (eg, dedicated extension/access study, access program, as applicable) at the time of the end of study and in accordance with local regulations.

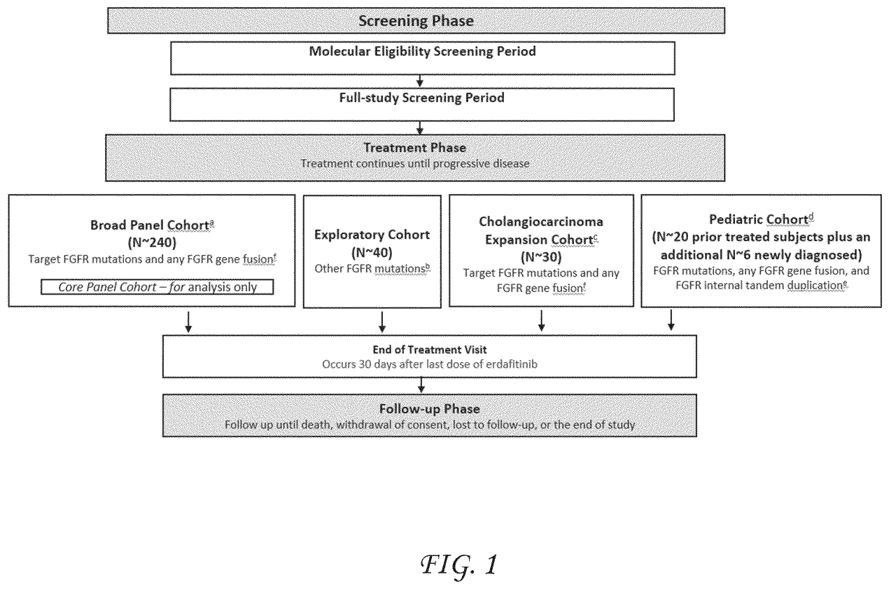

A schematic overview of the study is provided in FIG. 1.

### Study Overview

Number of Subjects: Approximately 280 subjects ≥12 years of age with FGFR genetic alterations are enrolled in the Broad Panel Cohort (240 subjects) and the Exploratory Cohort (40 subjects). An additional, approximately 30 subjects are enrolled in the Cholangiocarcinoma Expansion Cohort. The Pediatric Cohort (approximately 26 subjects) consisting of children or adolescent subjects ≥6 to <18 years of age with locally advanced or metastatic solid tumors will enroll 20 children or adolescent subjects who have progressed following prior therapies and who have no acceptable standard therapies, and approximately 6 additional children or adolescent subjects who have a newly diagnosed solid tumor (treatment naïve) and who have no acceptable standard therapies. The main requirements for prior treatment consist of at least one prior line of therapy in the metastatic setting and exhaustion of standard therapeutic options, i.e., there are no standard of care options that have shown meaningful clinical benefit for the relevant underlying histology and line of therapy, or the subject is unable to tolerate the therapy. Adolescent subjects enrolled in the Broad Panel Cohort (≥12 to <18 years) are analyzed as part the Broad Panel Cohort and the Pediatric Cohort.

Intervention Groups and Duration: Erdafitinib was provided as a tablet for oral administration. Subjects took erdafitinib orally once daily for 21 days on a 21-day cycle until disease progression, intolerable toxicity, withdrawal of consent, or decision by the investigator to discontinue treatment. Adults (aged 18 years and older for dosing purposes) and adolescent subjects aged 15 to <18 years started with an erdafitinib dose of 8 mg with possible up-titration to 9 mg based on Cycle 1 Day 14 serum phosphate levels. Each dose was taken approximately at the same time each day, with or without food. Adolescent subjects aged ≥12 to <15 years started with an erdafitinib dose of 5 mg with possible up-titration to 6 mg or further to 8 mg based on Cycle 1 Day 14 and Cycle 2 Day 7 serum phosphate levels. Children aged 6 to <12 years start with an erdafitinib dose of 3 mg with possible up-titration to 4 mg or further to 5 mg based on Cycle 1 Day 14 and Cycle 2 Day 7 serum phosphate levels.

Inclusion Criteria: Each subject satisfied all of the following criteria to be enrolled:
1. ≥6 years of age.
2. Histologic demonstration of an unresectable, locally advanced, or metastatic solid tumor malignancy bearing an FGFR mutation or fusion, as determined by local* or central laboratory screening.

*Locally performed or commercial testing results from tissue or blood with NGS tests, direct digital counting methods, or the Qiagen Therascreen® FGFR RT-PCR test performed in Clinical Laboratory Improvement Amendments (CLIA)-certified or regional equivalent laboratories.

### Molecular Criteria for Broad Panel Cohort:

Subjects with target FGFR mutations or any** FGFR gene fusions are eligible for enrollment in the Broad Panel Cohort. Subjects with other FGFR mutations*** not captured in the Broad Panel Cohort are eligible for enrollment in the Exploratory Cohort.

### Molecular Criteria for Pediatric Cohort:

Subjects with any FGFR mutation*** (exclusive of FGFR valine gatekeeper and resistance alterations defined in the Exclusion Criteria) or any** FGFR gene fusions, or FGFR duplication**** are eligible for enrollment in the Pediatric Cohort.

**FGFR Fusion Specifications:
Have a report suggesting the presence of an intact FGFR kinase domain
FGFR fusion with a 3-prime partner (FGFR gene is listed first, eg FGFR-GENE or FGFR3-TACC3):
   The FGFR portion of the fusion must involve exon 17 or greater (≥17)
FGFR fusion with a 5-primer partner (Partner gene is listed first and FGFR gene is second, eg GENE-FGFR or KLK2-FGFR2):
The FGFR portion of the fusion must involve less than or equal to exon 11 (≤11) and have a named FGFR fusion partner gene (self-fusions or rearrangements, e.g., FGFR-FGFR, are not eligible) (Broad Panel Cohort only)
*** Mutations in this study are defined as protein-coding single nucleotide variant (SNV) and insertions or deletions (indels). Copy number gains or gene-level amplifications are not eligible. FGFR mutations annotated as germline in local reports, or subjects presenting with a hereditary condition/disorder associated with a germline FGFR mutation are not eligible for enrollment in the absence of a qualifying FGFR mutation or fusion. Note, testing for germline mutations is not required for this study
**** For Pediatric Cohort Only: Intragenic duplication of the FGFR kinase domain (FGFR-FGFR) if one breakpoint is located within intron 8 through exon 11 and the other breakpoint is in intron 17 through intron 18 (including 3′ UTR). Copy number gains or gene-level amplifications are not eligible
3. Measurable disease according to Response Evaluation Criteria in Solid Tumors (RECIST) version 1.1 or Response Assessment in Neuro-Oncology (RANO) for primary brain tumors.
4. Subject must have received at least one prior line of systemic therapy in the advanced, unresectable, or metastatic setting, or is a child or adolescent subject with a newly diagnosed solid tumor and no acceptable standard therapies.
5. Subject does not have standard of care options that have shown meaningful clinical benefit for the relevant underlying histology and line of therapy or the subject is unable to tolerate the therapy.
6. Documented progression of disease, defined as any progression that requires a change in treatment, prior to full study screening.
7. Toxicities from previous anticancer therapies should have resolved to baseline levels or to Grade 1 or less except for alopecia, peripheral neuropathy, and Grade 2 laboratory values eligible per Inclusion Criterion 9.
8. For adults (≥18 years of age), Eastern Cooperative Oncology Group (ECOG) performance status Grade 0 or 1. For children and adolescents (≥6 to <16 years of age), Lansky Score of ≥80. For adolescents (≥16 to <18 years of age), Karnofsky Score of ≥80.
9. Adequate bone marrow, liver, and renal function. Bone marrow function (without the support of cytokines or erythropoiesis-stimulating agent transfusions in preceding 2 weeks): (a) Absolute neutrophil count (ANC) ≥1,000/mm3; (b) Platelet count ≥75,000/mm3; and (c) Hemoglobin ≥8.0 g/dL. Liver function: (a) Total bilirubin ≤1.5×institutional upper limit of normal (ULN) or direct bilirubin ≤ULN for subjects with total bilirubin levels ≥1.5×ULN; and (b) alanine aminotransferase (ALT) and aspartate aminotransferase (AST)≤2.5× institutional ULN or ≤5× institutional ULN for subjects with liver metastases. Renal function: Creatinine clearance >30 mL/min/1.73 m2 either directly measured via 24-hour urine collection or calculated using the Cockcroft-Gault formula for adult subjects or the CKiD (Chronic Kidney Disease in Children) Schwartz formula for children and adolescent subjects (≥6<18 years of age). Phosphate: <ULN within 14 days of treatment and prior to Cycle 1 Day 1 (medical management allowed).
10. Subjects must sign an informed consent form (or their legally acceptable representative must sign) indicating that the subject understands the nature, significance, and purpose of the study, and procedures required for the study, and consequence of the study; and is willing to participate in the study. For children and adolescent subjects, parent(s) (preferably both if available or as per local requirements) (or their legally acceptable representative) must sign an ICF indicating that the subject participant understands the purpose of, and procedures required for, the study and is willing to allow the child to participate in the study. Assent is also required of children and adolescent subjects.
11. A female of childbearing potential must have a negative pregnancy test (β-human chorionic gonadotropin [hCG]) at Screening (urine or serum).
12. Contraceptive use by male or female subjects should be consistent with local regulations regarding the use of contraceptive methods for subject participating in clinical studies.

Exclusion Criteria: Subjects who met any of the following criteria were excluded from participating in the study.
1. Has had prior chemotherapy, targeted therapy, or treatment with an investigational anticancer agent within 30 days or <5 half-lives of the agent (whichever is longer) and up to a maximum of 30 days before the first dose of erdafitinib. Has had prior immunotherapy within 30 days before the first dose of erdafitinib and/or has an ongoing Grade ≥2 immunotherapy-related toxicity.
2. The known* presence of FGFR valine gatekeeper and resistance alterations. Mutations in the following positions: FGFR1 V561; FGFR2 V564; FGFR3 V555; FGFR4 V550; FGFR1 N546; FGFR2 N549; FGFR3 N540 and FGFR4 N535. * Observation of a gatekeeper/resistance alteration in the local or central report. If the local test does not screen for all four FGFRs, e.g., FGFR4, the local report remains evaluable for molecular screening.
3. For non-small cell lung cancer (NSCLC) subjects only—pathogenic somatic mutations or gene fusions in EGFR* or BRAF V600E, KRAS, or any gene fusions in the following genes: ALK, ROS1, or NTRK. * Assessment of these genes may be performed per institutional standard and do not have to be assessed via NGS. For colorectal subjects only—pathogenic somatic mutations in BRAF, KRAS, NRAS and PIK3CA.
4. Histologic demonstration of urothelial carcinoma.
5. Hematologic malignancy (i.e., myeloid and lymphoid neoplasms).
6. Active malignancies other than for disease requiring therapy.
7. Symptomatic central nervous system metastases (except for subjects with primary CNS tumors).
8. Received prior selective FGFR inhibitor treatment.
9. Known allergies, hypersensitivity, or intolerance to erdafitinib or its excipients.
10. Current central serous retinopathy (CSR) or retinal pigment epithelial detachment of any grade.
11. History of uncontrolled cardiovascular disease including: (a) Unstable angina, myocardial infarction, ventricular fibrillation, Torsades de Pointes, cardiac arrest, or known congestive heart failure Class III-V within the preceding 3 months; cerebrovascular accident or transient ischemic attack within the preceding 3 months; (b) QTc prolongation (Fridericia: QTc >480 milliseconds; or for children and adolescent subjects, Bazett: QTc >440 milliseconds).
12. Known history of AIDS (human immunodeficiency virus (HIV) infection), unless the subject has been on a stable anti-retroviral therapy regimen for the last 6 months or more, has had no opportunistic infections in the last 6 months, and has CD4 count >350.
13. Evidence of active hepatitis B or C infection (for example, subjects with history of hepatitis C infection but normal hepatitis C virus polymerase chain reaction test and subjects with hepatitis B with positive HbsAg antibody are allowed).
14. Not recovered from reversible toxicity of prior anticancer therapy (except toxicities which are not clinically significant such as alopecia, skin discoloration, neuropathy, hearing loss).
15. Impaired wound healing capacity defined as skin/decubitus ulcers, chronic leg ulcers, known gastric ulcers, or unhealed incisions.
16. Major surgery within 4 weeks before first dose of erdafitinib.
17. Palliative radiation to the target lesion within 2 weeks before the first dose of erdafitinib.
18. Pregnant, or breast-feeding, or planning to become pregnant while enrolled in this study or within 3 months after the last dose of drug.
19. Plans to father a child while enrolled in this study or within 3 months after the last dose of drug.
20. Any condition for which, in the opinion of the investigator, participation would not be in the best interest of the subject (e.g., compromise the well-being) or that could prevent, limit, or confound the protocol-specified assessments. Non-limiting examples include ongoing active infection requiring systemic therapy and uncontrolled ongoing medical conditions.

FGFR Markers: Subjects with target FGFR mutations or any FGFR gene fusions were eligible for enrollment in the Broad Panel Cohort. Subjects with other FGFR mutations not captured in the Broad Panel Cohort were eligible for enrollment in the Exploratory Cohort. FGFR gene fusions must have an intact FGFR kinase domain. FGFR gene identifiers and sequences are provided in Table 5. This extension of molecular ability to include any FGFR gene fusion with an intact FGFR kinase domain is based on clinical experience in cholangiocarcinoma and other tumor types where clinical responses to erdafitinib were observed in patients with novel gene fusions.

TABLE 5

| | | | Kinase | Kinase | |
|---|---|---|---|---|---|
| | | RefseqID | Domain AA | Domain | |
| Gene | Ensembl ID | mRNA | Position¹ | Exons¹ | Amino acid sequence |
| FGFR1 | ENST00000447712 | NM_023110.3 | 478 → 754 | Exons | MWSWKCLLFWAV |
| | | | | 11-17 | LVTATLCTARPSPTL |
| | | | | | PEQAQPWGAPVEV |
| | | | | | ESFLVHPGDLLQLR |
| | | | | | CRLRDDVQSINWLR |
| | | | | | DGVQLAESNRTRIT |
| | | | | | GEEVEVQDSVPADS |
| | | | | | GLYACVTSSPSGSD |
| | | | | | TTYFSVNVSDALPS |
| | | | | | SEDDDDDDDSSSEE |
| | | | | | KETDNTKPNRMPV |
| | | | | | APYWTSPEKMEKK |
| | | | | | LHAVPAAKTVKFK |
| | | | | | CPSSGTPNPTLRWL |
| | | | | | KNGKEFKPDHRIGG |
| | | | | | YKVRYATWSIIMDS |
| | | | | | VVPSDKGNYTCIVE |
| | | | | | NEYGSINHTYQLDV |
| | | | | | VERSPHRPILQAGLP |
| | | | | | ANKTVALGSNVEF |
| | | | | | MCKVYSDPQPHIQ |
| | | | | | WLKHIEVNGSKIGP |
| | | | | | DNLPYVQILKTAGV |
| | | | | | NTTDKEMEVLHLR |
| | | | | | NVSFEDAGEYTCLA |
| | | | | | GNSIGLSHHSAWLT |
| | | | | | VLEALEERPAVMTS |
| | | | | | PLYLEIIIYCTGAFLI |
| | | | | | SCMVGSVIVYKMK |
| | | | | | SGTKKSDFHSQMA |
| | | | | | VHKLAKSIPLRRQV |
| | | | | | TVSADSSASMNSGV |
| | | | | | LLVRPSRLSSSGTPM |
| | | | | | LAGVSEYELPEDPR |
| | | | | | WELPRDRLVLGKPL |
| | | | | | GEGCFGQVVLAEAI |
| | | | | | GLDKDKPNRVTKV |
| | | | | | AVKMLKSDATEKD |
| | | | | | LSDLISEMEMMKMI |
| | | | | | GKHKNIINLLGACT |
| | | | | | QDGPLYVIVEYASK |
| | | | | | GNLREYLQARRPPG |
| | | | | | LEYCYNPSHNPEEQ |
| | | | | | LSSKDLVSCAYQVA |
| | | | | | RGMEYLASKKCIHR |
| | | | | | DLAARNVLVTEDN |
| | | | | | VMKIADFGLARDIH |
| | | | | | HIDYYKKTTNGRLP |
| | | | | | VKWMAPEALFDRI |
| | | | | | YTHQSDVWSFGVL |
| | | | | | LWEIFTLGGSPYPG |
| | | | | | VPVEELFKLLKEGH |
| | | | | | RMDKPSNCTNELY |
| | | | | | MMMRDCWHAVPS |
| | | | | | QRPTFKQLVEDLDR |
| | | | | | IVALTSNQEYLDLS |
| | | | | | MPLDQYSPSFPDTR |
| | | | | | SSTCSSGEDSVFSHE |
| | | | | | PLPEEPCLPRHPAQL |
| | | | | | ANGGLKRR |
| | | | | | (SEQ ID NO: 1) |
| FGFR2 | ENST00000358487 | NM_000141.5 | 481 → 757 | Exons | MVSWGRFICLVVVT |
| | | | | 11-17 | MATLSLARPSFSLV |
| | | | | | EDTTLEPEEPPTKYQ |
| | | | | | ISQPEVYVAAPGESL |
| | | | | | EVRCLLKDAAVISW |
| | | | | | TKDGVHLGPNNRT |
| | | | | | VLIGEYLQIKGATPR |
| | | | | | DSGLYACTASRTVD |
| | | | | | SETWYFMVNVTDAI |
| | | | | | SSGDDEDDTDGAED |
| | | | | | FVSENSNNKRAPY |
| | | | | | WTNTEKMEKRLHA |
| | | | | | VPAANTVKFRCPAG |
| | | | | | GNPMPTMRWLKNG |
| | | | | | KEFKQEHRIGGYKV |
| | | | | | RNQHWSLIMESVVP |
| | | | | | SDKGNYTCVVENE |
| | | | | | YGSINHTYHLDVVE |
| | | | | | RSPHRPILQAGLPAN |
| | | | | | ASTVVGGDVEFVC |
| | | | | | KVYSDAQPHIQWIK |
| | | | | | HVEKNGSKYGPDG |
| | | | | | LPYLKVLKAAGVN |
| | | | | | TTDKEIEVLYIRNVT |
| | | | | | FEDAGEYTCLAGNS |
| | | | | | IGISFHSAWLTVLPA |
| | | | | | PGREKEITASPDYLE |
| | | | | | IAIYCIGVFLIACMV |
| | | | | | VTVILCRMKNTTKK |
| | | | | | PDFSSQPAVHKLTK |
| | | | | | RIPLRRQVTVSAESS |
| | | | | | SSMNSNTPLVRITTR |
| | | | | | LSSTADTPMLAGVS |
| | | | | | EYELPEDPKWEFPR |
| | | | | | DKLTLGKPLGEGCF |
| | | | | | GQVVMAEAVGIDK |
| | | | | | DKPKEAVTVAVKM |
| | | | | | LKDDATEKDLSDLV |
| | | | | | SEMEMMKMIGKHK |
| | | | | | NIINLLGACTQDGPL |
| | | | | | YVIVEYASKGNLRE |
| | | | | | YLRARRPPGMEYSY |
| | | | | | DINRVPEEQMTFKD |
| | | | | | LVSCTYQLARGME |
| | | | | | YLASQKCIHRDLAA |
| | | | | | RNVLVTENNVMKI |
| | | | | | ADFGLARDINNIDY |
| | | | | | YKKTTNGRLPVKW |
| | | | | | MAPEALFDRVYTH |
| | | | | | QSDVWSFGVLMWE |
| | | | | | IFTLGGSPYPGIPVE |
| | | | | | ELFKLLKEGHRMD |
| | | | | | KPANCTNELYMMM |
| | | | | | RDCWHAVPSQRPTF |
| | | | | | KQLVEDLDRILTLT |
| | | | | | TNEEYLDLSQPLEQ |
| | | | | | YSPSYPDTRSSCSSG |
| | | | | | DDSVFSPDPMPYEP |
| | | | | | CLPQYPHINGSVKT |
| | | | | | (SEQ ID NO: 2) |
| FGFR3 | ENST00000260795 | NM_000142.4 | 478 → 754 | Exons | MGAPACALALCVA |
| | | | | 11-17 | VAIVAGASSESLGT |
| | | | | | EQRVVGRAAEVPGP |
| | | | | | EPGQQEQLVFGSGD |
| | | | | | AVELSCPPPGGGPM |
| | | | | | GPTVWVKDGTGLV |
| | | | | | PSERVLVGPQRLQV |
| | | | | | LNASHEDSGAYSCR |
| | | | | | QRLTQRVLCHFSVR |
| | | | | | VTDAPSSGDDEDGE |
| | | | | | DEAEDTGVDTGAP |
| | | | | | YWTRPERMDKKLL |
| | | | | | AVPAANTVRFRCPA |
| | | | | | AGNPTPSISWLKNG |
| | | | | | REFRGEHRIGGIKLR |
| | | | | | HQQWSLVMESVVP |
| | | | | | SDRGNYTCVVENKF |
| | | | | | GSIRQTYTLDVLERS |
| | | | | | PHRPILQAGLPANQ |
| | | | | | TAVLGSDVEFHCKV |
| | | | | | YSDAQPHIQWLKH |
| | | | | | VEVNGSKVGPDGTP |
| | | | | | YVTVLKTAGANTT |
| | | | | | DKELEVLSLHNVTF |
| | | | | | EDAGEYTCLAGNSI |
| | | | | | GFSHHSAWLVVLPA |
| | | | | | EEELVEADEAGSVY |
| | | | | | AGILSYGVGFFLFIL |
| | | | | | VVAAVTLCRLRSPP |
| | | | | | KKGLGSPTVHKISR |
| | | | | | FPLKRQVSLESNAS |
| | | | | | MSSNTPLVRIARLSS |
| | | | | | GEGPTLANVSELEL |
| | | | | | PADPKWELSRARLT |
| | | | | | LGKPLGEGCFGQVV |
| | | | | | MAEAIGIDKDRAAK |
| | | | | | PVTVAVKMLKDDA |
| | | | | | TDKDLSDLVSEME |
| | | | | | MMKMIGKHKNIINL |
| | | | | | LGACTQGGPLYVLV |
| | | | | | EYAAKGNLREFLRA |
| | | | | | RRPPGLDYSFDTCK |
| | | | | | PPEEQLTFKDLVSC |
| | | | | | AYQVARGMEYLAS |
| | | | | | QKCIHRDLAARNVL |
| | | | | | VTEDNVMKIADFGL |
| | | | | | ARDVHNLDYYKKT |
| | | | | | TNGRLPVKWMAPE |
| | | | | | ALFDRVYTHQSDV |
| | | | | | WSFGVLLWEIFTLG |
| | | | | | GSPYPGIPVEELFKL |
| | | | | | LKEGHRMDKPANC |
| | | | | | THDLYMIMRECWH |
| | | | | | AAPSQRPTFKQLVE |
| | | | | | DLDRVLTVTSTDEY |
| | | | | | LDLSAPFEQYSPGG |
| | | | | | QDTPSSSSSGDDSVF |
| | | | | | AHDLLPPAPPSSGGS |
| | | | | | RT (SEQ ID NO: 3) |
| FGFR4 | ENST00000292408 | NM_002011.5 | 467→ 743 | Exons | MRLLLALLGVLLSV |
| | | | | 11-17 | PGPPVLSLEASEEVE |
| | | | | | LEPCLAPSLEQQEQ |
| | | | | | ELTVALGQPVRLCC |
| | | | | | GRAERGGHWYKEG |
| | | | | | SRLAPAGRVRGWR |
| | | | | | GRLEIASFLPEDAGR |
| | | | | | YLCLARGSMIVLQN |
| | | | | | LTLITGDSLTSSNDD |
| | | | | | EDPKSHRDPSNRHS |
| | | | | | YPQQAPYWTHPQR |
| | | | | | MEKKLHAVPAGNT |
| | | | | | VKFRCPAAGNPTPTI |
| | | | | | RWLKDGQAFHGEN |
| | | | | | RIGGIRLRHQHWSL |
| | | | | | VMESVVPSDRGTYT |
| | | | | | CLVENAVGSIRYNY |
| | | | | | LLDVLERSPHRPILQ |
| | | | | | AGLPANTTAVVGSD |
| | | | | | VELLCKVYSDAQPH |
| | | | | | IQWLKHIVINGSSFG |
| | | | | | ADGFPYVQVLKTA |
| | | | | | DINSSEVEVLYLRN |
| | | | | | VSAEDAGEYTCLAG |
| | | | | | NSIGLSYQSAWLTV |
| | | | | | LPEEDPTWTAAAPE |
| | | | | | ARYTDIILYASGSLA |
| | | | | | LAVLLLLAGLYRGQ |
| | | | | | ALHGRHPRPPATVQ |
| | | | | | KLSRFPLARQFSLES |
| | | | | | GSSGKSSSSLVRGV |
| | | | | | RLSSSGPALLAGLV |
| | | | | | SLDLPLDPLWEFPR |
| | | | | | DRLVLGKPLGEGCF |
| | | | | | GQVVRAEAFGMDP |
| | | | | | ARPDQASTVAVKM |
| | | | | | LKDNASDKDLADL |
| | | | | | VSEMEVMKLIGRH |
| | | | | | KNIINLLGVCTQEGP |
| | | | | | LYVIVECAAKGNLR |
| | | | | | EFLRARRPPGPDLSP |
| | | | | | DGPRSSEGPLSFPVL |
| | | | | | VSCAYQVARGMQY |
| | | | | | LESRKCIHRDLAAR |
| | | | | | NVLVTEDNVMKIA |
| | | | | | DFGLARGVHHIDYY |
| | | | | | KKTSNGRLPVKWM |
| | | | | | APEALFDRVYTHQS |
| | | | | | DVWSFGILLWEIFTL |
| | | | | | GGSPYPGIPVEELFS |
| | | | | | LLREGHRMDRPPHC |
| | | | | | PPELYGLMRECWH |
| | | | | | AAPSQRPTFKQLVE |
| | | | | | ALDKVLLAVSEEYL |
| | | | | | DLRLTFGPYSPSGG |
| | | | | | DASSTCSSSDSVFSH |
| | | | | | DPLPLGSSSFPFGSG |
| | | | | | VQT (SEQ ID NO: 4) |

¹Kinase domains defined by Protein Families database (Pfam) annotations from NCBI
Entrez Gene. Exons of the RefSeq transcript are inclusive of the kinase domain. Exon
boundaries defining the kinase domain are equivalent to NM_015850.

Panel Cohorts: The alterations eligible for inclusion in the Broad Panel Cohort include target FGFR mutations as provided in Example 1 and any FGFR fusion. The Broad Panel Cohort represents the primary cohort of interest for analysis. Populations for analysis of the Broad Panel Cohort are specified below:

The Treated Population will consist of all subjects who receive at least 1 dose of study drug. The Treated Population is the primary population for efficacy and safety analyses. The Response-evaluable Population will include all subjects who satisfy the following criteria: Met all eligibility criteria for the study; received at least 1 dose of study drug; and had a baseline and at least 1 adequate post-treatment radiological disease evaluation, or had clinical signs or symptoms of disease progression, or died prior to the first posttreatment disease evaluation (these subjects will be considered non-responders). Adequate disease assessment is defined as having enough evidence to indicate that progression has or has not occurred.

FGFR alteration frequencies for 8 of the most frequently observed pediatric tumors were assessed utilizing the FoundationInsights database.

Subjects enrolled in Exploratory Cohort who receive at least 1 dose of study drug, will be evaluated for efficacy and safety as the exploratory analysis.

The Treated Population will be used to summarize the study population and characteristics, efficacy, and PRO data; the efficacy and safety analyses will be conducted in the Treated Population in the Broad Panel Cohort. The Response-evaluable Population will be used for the interim analysis, and supportive efficacy analysis on key endpoints such as ORR and DOR.

Evaluations: Assessment of response was performed according to RECIST, version 1.1, or RANO by the Independent Review Committee (IRC) and investigators. Pharmacokinetic assessments (plasma concentrations of erdafitinib and alpha-i-acid glycoproteins, total protein, and fraction unbound, if required, using venous blood samples), biomarker assessments (molecular screening to determine eligibility for the study; and exploratory DNA, RNA, and protein analyses using archival or fresh biopsy tissue and blood (ctDNA) for exploratory research), patients' health-related quality of life (QoL) assessments, and safety assessments (including adverse event [AE] reports and results of vital sign measurements, electrocardiograms [ECGs], physical examinations, clinical laboratory tests, performance status assessment, and ophthalmologic examinations) were also conducted. Additional safety assessments for children and adolescents include radiographic (growth plate assessment and bone age) imaging, DEXA scan for bone densitometry, and clinical laboratory tests for thyroid stimulation hormone (TSH), total triiodothyronine (T3), and free thyroxine (T4) and insulin-like growth factor 1 (IGF-1) will also be conducted.

Statistical Methods: For the Broad Panel Cohort, the primary endpoint was overall response rate (ORR) based on RECIST v1.1. or RANO as assessed by the IRC and was calculated with a 95% 2-sided exact confidence interval (CI). The primary endpoint was analyzed using data from the Treated Population (defined as all subjects who receive at least 1 dose of study drug) in the Broad Panel Cohort and the Core Panel Cohort. An error-spending function approach was used to split the significance level for the Broad Panel Cohort and the Core Panel Cohort.

The secondary endpoints include ORR by investigator assessment, duration of response (DOR), disease control rate (DCR), clinical benefit rate (CBR), progression-free survival (PFS), overall survival (OS), pharmacokinetic (PK) exposure parameters, incidence and severity of adverse events (Aes), and patient-reported outcomes (PROs). The ORR assessed by the investigator was analyzed in the same way as the ORR assessed by the IRC. The distributions of DOR, PFS and OS are summarized using Kaplan-Meier estimates and the estimated median is reported along with a 95% CI. The PRO assessments are analyzed with descriptive summaries (i.e., mean, standard deviation including change from baseline) at each assessment time point. The Pediatric Cohort has the same primary and secondary endpoints as the Broad Panel Cohort; subjects in the Pediatric Cohort are evaluated separated from the Broad Panel Cohort. The Cholangiocarcinoma Expansion Cohort is evaluated separated from the Broad Panel Cohort.

Three interim futility analyses were planned when 30%, 50%, and 70% of the subjects in the Broad Panel Cohort (i.e., approximately 60, 100, and 140 subjects) have been treated and are considered response-evaluable, irrespective of the tumor histologies and the distribution among the tumor histologies. The interim analyses for futility is based on the primary endpoint (ORR) using a Bayesian hierarchical model (BHM), implemented in FACTS v6.2 Enrichment Design—Dichotomous. In addition, an interim efficacy analysis was conducted for the Broad Panel Cohort at the same time as the second interim futility analysis. The primary analysis will be based on the Treated Population of the BPC conducted 6 months after approximately 200 response-evaluable subjects have been treated.

### Up-Titration Guidelines

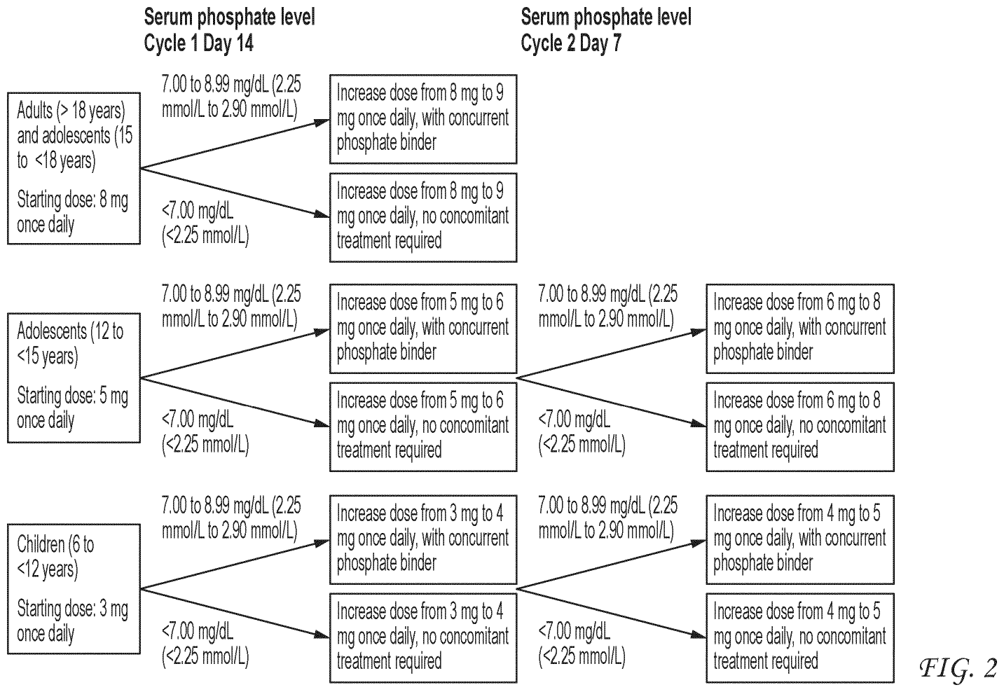

Instructions for up-titration of erdafitinib, in the absence of erdafitinib related toxicity, based on the serum phosphate level at Cycle 1 Day 14 are provided below and in FIG. 2. For children aged 6 to <12 years and adolescents aged 12 to <15 years an additional blood sample will be drawn at Cycle 2 Day 7 for determination of the serum phosphate level and the need for dose modification.

Subjects with a serum phosphate level higher than and including 9.00 mg/dL (>2.91 mmol/L) will withhold erdafitinib treatment, with at least weekly assessment of serum phosphate until it returns to less than 7.00 mg/dL (<2.25 mmol/L) while initiating treatment with a phosphate binder such as sevelamer.

### Adult Subjects and Adolescent Subjects Aged 15 to <18 Years

Adult subjects and adolescent subjects aged 15 to <18 years with a serum phosphate level between 7.00 to 8.99 mg/dL (2.25 mmol/L to 2.90 mmol/L) on Cycle 1 Day 14 will increase the erdafitinib dose from 8 mg once daily to 9 mg once daily, while concurrently initiating treatment with a phosphate binder such as sevelamer.

Adult subjects and adolescent subjects aged 15 to <18 years with a serum phosphate level less than 7.00 mg/dL (<2.25 mmol/L) will increase the erdafitinib dose to 9 mg once daily on Cycle 1 Day 14. No concomitant treatment is required for these subjects

### Adolescent Subjects Aged 12 to <15 Years

Adolescent subjects aged 12 to <15 years with a serum phosphate level between 7.00 to 8.99 mg/dL (2.25 mmol/L to 2.90 mmol/L) will increase the erdafitinib dose from 5 mg once daily to 6 mg once daily on Cycle 1 Day 14 or Cycle 2 Day 7, and further from 6 mg once daily to 8 mg once daily on Cycle 2 Day 7 (for those already up-titrated to 6 mg on Cycle 1 Day 14), while concurrently initiating treatment with a phosphate binder such as sevelamer. This 2-step up-titration is step-wise, i.e., no subjects will be allowed to directly up-titrate from 5 mg to 8 mg.

Adolescent subjects aged 12 to <15 years with a serum phosphate level less than 7.00 mg/dL (<2.25 mmol/L) will increase the erdafitinib dose from 5 mg once daily to 6 mg once daily on Cycle 1 Day 14 or Cycle 2 Day 7, and further from 6 mg once daily to 8 mg once daily on Cycle 2 Day 7 (for those already up-titrated to 6 mg on Cycle 1 Day 14). This 2-step uptitration is step-wise, i.e., no subjects will be allowed to directly up-titrate from 5 mg to 8 mg. No concomitant treatment is required

### Children Aged 6 to <12 Years

Children aged 6 to <12 years with a serum phosphate level between 7.00 to 8.99 mg/dL (2.25 mmol/L to 2.90 mmol/L) will increase the erdafitinib dose from 3 mg once daily to 4 mg once daily on Cycle 1 Day 14 or Cycle 2 Day 7, and further from 4 mg once daily to 5 mg once daily on Cycle 2 Day 7 (for those already up-titrated to 4 mg on Cycle 1 Day 14), while concurrently initiating treatment with a phosphate binder such as sevelamer. This 2 step up-titration is step-wise, i.e., no subjects will be allowed to directly up-titrate from 3 mg to 5 mg.

Children aged 6 to <12 years with a serum phosphate level less than 7.00 mg/dL (<2.25 mmol/L) will increase the erdafitinib dose from 3 mg once daily to 4 mg once daily on Cycle 1 Day 14 or Cycle 2 Day 7, and further from 4 mg once daily to 5 mg once daily on Cycle 2 Day 7 (for those already up-titrated to 4 mg on Cycle 1 Day 14). This 2-step up-titration is step-wise, ie, no subjects will be allowed to directly up-titrate from 3 mg to 5 mg. No concomitant treatment is required.

If a dose is missed, then it can be taken up to 6 hours after the scheduled time; the subject may return to the normal schedule the following day. If it has been more than 6 hours since the missed dose, then that dose should be skipped, and the subject should continue treatment at the scheduled time the next day. If vomiting occurred with drug administration, no replacement dose will be taken and any such event that occurs up to 4 hours following dose administration must be recorded on the electronic case report form (eCRF).

The exposure of erdafitinib may increase by 50% in subjects with the CYP2C9*3/*3 genotype, estimated to be 0.4% to 3% of the population among various ethnic groups. Therefore, increased adverse reactions are monitored in subjects who are known or suspected to have CYP2C9*3/*3 genotype. Dose titration is guided by serum phosphate levels in all subjects irrespective of genotype; therefore, the implications of higher exposures of erdafitinib including safety may be addressed.

### Objectives and Endpoints.

The primary and secondary objectives and endpoints are provided in Table 6.

TABLE 6
Objectives and Endpoints

| | |
|---|---|
| Objectives | Endpoints |

Primary Objective (Broad Panel Cohort and Core Panel Cohort)

| | |
|---|---|
| To evaluate the efficacy of erdafitinib in | The proportion of subjects who achieve a |
| terms of ORR as assessed by the IRC in | complete response (CR) or partial response |
| subjects with advanced solid tumors with | (PR) based on RECIST v1.1. or RANO as |
| target FGFR mutations and any gene | assessed by IRC |
| fusions (Broad Panel Cohort), or in a pre- | |
| specified subgroup of subjects with a | |
| selected panel of FGFR markers (Core | |
| Panel Cohort), or in both cohorts | |

Primary Objective (Pediatric Cohort)

| | |
|---|---|
| To evaluate the efficacy of erdafitinib in terms | The proportion of subjects who achieve a CR |
| of ORR as assessed by the IRC in pediatric | or PR based on RECIST v1.1. or RANO as |
| subjects with advanced solid tumors with | assessed by IRC |
| FGFR mutations, any gene fusions and any | |
| FGFR duplication (Pediatric Cohort), | |
| including adolescent subjects with target | |
| FGFR mutations and any gene fusions | |

Secondary Objectives (All Cohorts)

| | |
|---|---|
| To evaluate the efficacy of erdafitinib, in | The proportion of subjects who achieve a |
| terms of the ORR, as assessed by | CR or PR based on RECIST v1.1. or |
| investigator | RANO as assessed by investigator |
| To evaluate the efficacy of erdafitinib in | DOR: the duration from the date of initial |
| terms of DOR | documentation of a response to the date of |
| | first documented evidence of progressive |
| | disease (or relapse for subjects who |
| | experience CR during the study) or death, |
| | whichever comes first. Data from subjects |
| | who are progression-free and alive or have |
| | unknown status will be censored at the last |
| | tumor assessment |
| To evaluate other measures of efficacy | DCR: the proportion of subjects with CR, |
| including DCR, CBR, PFS, and OS | PR or SD |
| | CBR: the proportion of subjects with CR, |
| | PR or durable SD (defined as duration of |
| | at least 4 months) |
| | PFS: the duration from the date of the first |
| | dose of study drug until the date of first |
| | documented evidence of progressive |
| | disease (or relapse for subjects who |
| | experience CR during the study) or death, |
| | whichever comes first. Data from subjects |
| | who are progression-free and alive or have |
| | unknown status will be censored at the last |
| | tumor assessment |
| | OS: measured from the date of first dose |
| | of study drug to the date of the subject's |
| | death. If the subject is alive or the vital |
| | status is unknown, the subject's data will |
| | be censored at the date the subject was last |
| | known to be alive |
| To evaluate erdafitinib PK | PK exposure parameters derived using |
| | existing population PK model. This |
| | endpoint include pediatrics. |
| To evaluate safety and tolerability of | Incidence and severity of Aes |
| erdafitinib | |
| To evaluate Health-Related Quality of Life | Change from baseline in patient-reported |
| | health status and physical functioning |
| | scales of the European Organisation for |
| | Research and Treatment of Cancer |
| | Quality-of-life Questionnaire Core 30 |
| | (EORTC-QLQ-C30; for subjects ≥18 |
| | years of age) or Pediatric Functional |
| | Assessment Of Cancer Therapy - Brain |
| | (Peds FACT-Br for subjects <18 years of |
| | age), Patient Global Impression of |
| | Symptom Severity (PGIS), Patient Global |
| | Impression of Change (PGIC), and |
| | European Quality of Life - 5 Dimensions- |
| | 5 Levels (EQ-5D-5L). |

Cholangiocarcinoma Expansion Cohort

| | |
|---|---|
| To evaluate the efficacy and safety of | Key efficacy endpoints will be evaluated |
| erdafitinib in subjects with | including ORR assessed by IRC/investigator, |
| cholangiocarcinoma with target FGFR | DOR, PFS, and OS |
| mutations and any gene fusions | Incidence and severity of Aes |

### Interim Analysis Results

These results are based on the following interim dosing schedule: Subjects took erdafitinib orally once daily for 21 days on a 21-day cycle until disease progression, intolerable toxicity, withdrawal of consent, or decision by the investigator to discontinue treatment. Adults (aged 18 years and older for dosing purposes) and adolescent subjects aged 15 to <18 years started with an erdafitinib dose of 8 mg with possible up-titration to 9 mg based on Cycle 1 Day 14 serum phosphate levels. Each dose was taken approximately at the same time each day, with or without food. Adolescent subjects aged 12 to <15 years started with an erdafitinib dose of 5 mg with possible up-titration to 6 mg or further to 8 mg based on Cycle 1 Day 7 and Cycle 1 Day 14 serum phosphate levels.

The following results are based on the following patient set: 5758 patients were molecularly screened (central or local) in 15 countries. 191 patients (3.3%) fulfilled primary analysis molecular eligibility criteria. 110 patients were enrolled. Median age was 57 years, and 19 patients (17.3%) were aged <40 years; distribution of males and females was even (Table 7). Among patients enrolled, 14 (12.7%) had central screening and 96 (87.3%) had local next-generation sequencing (NGS) reports (Table 7).

TABLE 7
Demographics and molecular testing
of the RAGNAR enrollment population

| Demographics of enrolled population | (N = 110) |
|---|---|
| Age, median (range), years | 57 (13-79) |
| Sex | |
| Male, n(%) | 55 (50.0) |
| Female, n(%) | 55 (50.0) |
| Molecular testing | |
| Centrally confirmed, n (%) | 14 (12.7) |
| Local NGS, n (%) | 96 (87.3) |

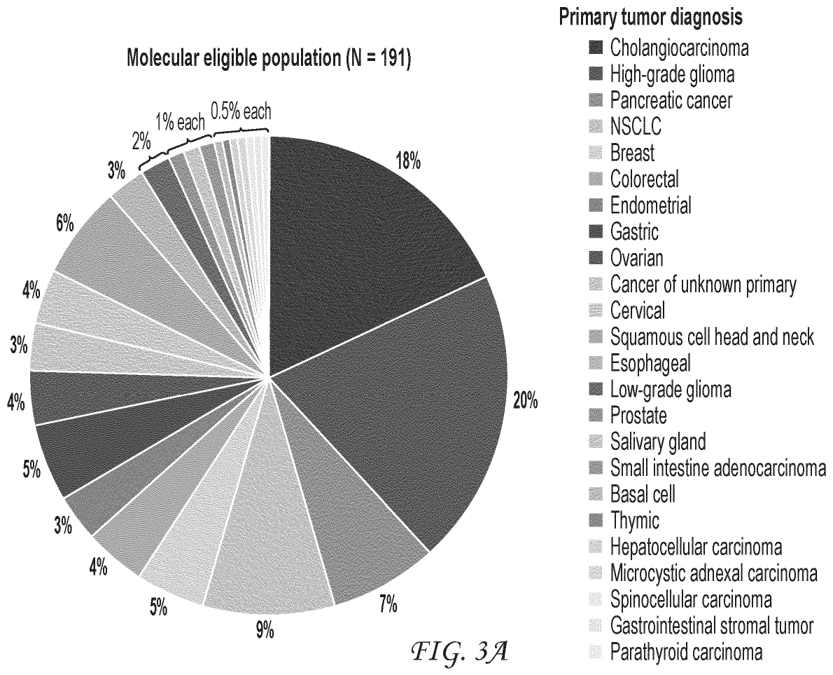

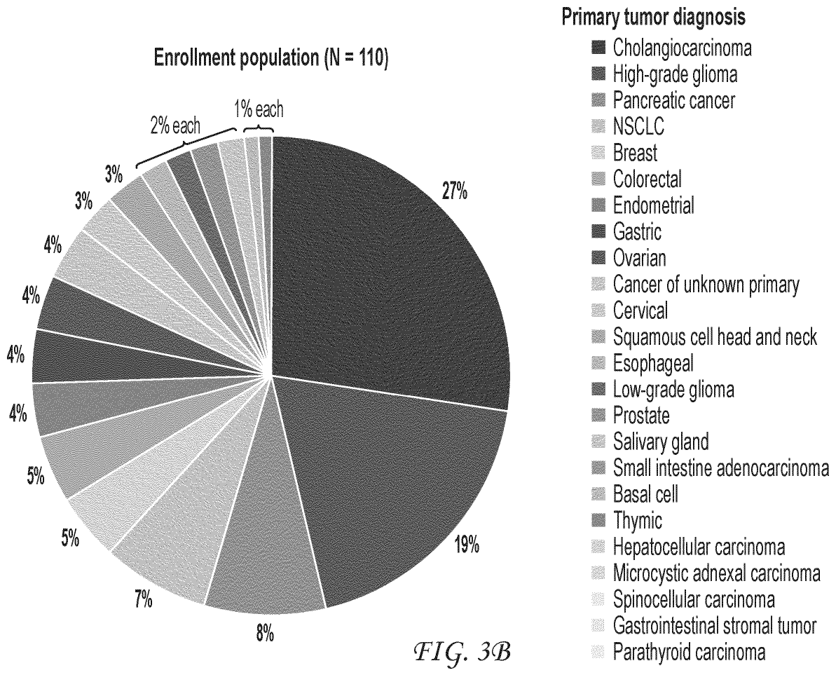

Eligible FGFR alterations were identified in 18 tumor types, including rare cancers (Table 8 and FIG. 3A and FIG. 3B).

TABLE 8

| | n (%) | Predominant Eligible | Representative FGFR |
|---|---|---|---|
| Cancer | (N = 110) | FGFR Alteration (%) | Variant(s) |
| Cholangiocarcinoma | 30 (27) | FGFR2 fusion (80) | FGFR2-BICC1 fusion |
| High-grade glioma | 21 (19) | FGFR3 fusion (95) | FGFR3-TACC3 fusion |
| Pancreatic | 9 (8) | FGFR2 fusion (78) | FGFR1-MTUS1 fusion |
| NSCLC | 8 (7) | FGFR3 fusion (63) | FGFR3-TACC3 fusion |
| Breast | 5 (5) | FGFR2 fusion (40) and | FGFR2-TBC1D4 fusion |
| | | mutation (40) | |
| Colorectal | 5 (5) | FGFR3 fusion (40) and | FGFR3-TACC3 fusion |
| | | mutation (40) | |
| Endometrial | 4 (4) | FGFR2 mutation (100) | FGFR2-C382R mutation |
| Gastric | 4 (4) | FGFR3 mutation (50) | FGFR3-TACC3 fusion |
| Ovarian | 4 (4) | FGFR2 fusion (50) | FGFR2-CLOCK fusion |
| Cancer of unknown | 4 (4) | FGFR2 fusion (50) | FGFR2-Y375C mutation |
| primary origin | | | |
| Cervical | 3 (3) | FGFR3 mutation (100) | FGFR3-S249C mutation |
| Squamous cell head | 3 (3) | FGFR3 fusion (67) | FGFR3-TACC3 fusion |
| and neck | | | |
| Esophageal | 2 (2) | FGFR3 fusion (50) and | FGFR3-R248C mutation |
| | | mutation (50) | |
| Low-grade glioma | 2 (2) | FGFR1 mutation (100) | FGFR1-K655E mutation |
| Prostate | 2 (2) | FGFR3 fusion (50) and | FGFR3-R248C mutation |
| | | mutation (50) | |
| Salivary gland | 2 (2) | FGFR2 mutation (50) | FGFR2-C382R mutation |
| Basal cell | 1 (1) | FGFR2 mutation (100) | FGFR2-S252L mutation |
| Thymic | 1 (1) | FGFR1 fusion (100) | IGSF3-FGFR1 fusion |

Table 9 shows single patient level FGFR alterations and the best overall response at interim analysis 2 (135 treated Broad Panel Cohort patients).

TABLE 9

| | | | | Best |
|---|---|---|---|---|
| | | Alteration | | overall |
| ID | Histology | Type | FGFR Alteration | response |
| 1 | Cholangiocarcinoma | Fusion | FGFR2-TBC1D4 | PR |
| 2 | Cholangiocarcinoma | Fusion | FGFR2-LGSN | SD |
| 3 | Cholangiocarcinoma | Fusion | FGFR2-TRA2B | PR |
| 4 | Cholangiocarcinoma | Fusion | FGFR2-BICC1 | PR |
| 5 | Cholangiocarcinoma | Fusion | FGFR2-BICC1 | PR |
| 6 | Cholangiocarcinoma | Fusion | FGFR2-BICC1 | SD |
| 7 | Cholangiocarcinoma | Fusion | FGFR2-BICC1 | SD |
| 8 | Cholangiocarcinoma | Fusion | FGFR2-NOL4 | SD |
| 9 | Cholangiocarcinoma | Mutation | FGFR2-C382R | SD |
| 10 | Cholangiocarcinoma | Fusion | FGFR2-BICC1 | PR |
| 11 | Cholangiocarcinoma | Fusion | FGFR2-PAWR | CR |
| 12 | Cholangiocarcinoma | Fusion | FGFR2-PDE3A | PD |
| 13 | Cholangiocarcinoma | Fusion | FGFR2-AHCYL1 | SD |
| 14 | Cholangiocarcinoma | Fusion | FGFR2-SYNPO2 | PR |
| 15 | Cholangiocarcinoma | Mutation | FGFR2-V395D | PD |
| 16 | Cholangiocarcinoma | Fusion | FGFR2-BICC1 | SD |
| 17 | Cholangiocarcinoma | Fusion | FGFR2-PAWR | PR |
| 18 | Cholangiocarcinoma | Fusion | FGFR2-ENOX1 | PR |
| 19 | Cholangiocarcinoma | Fusion | FGFR3-TACC3 | SD |
| 20 | Cholangiocarcinoma | Fusion | FGFR2-POC1B | SD |
| 21 | Cholangiocarcinoma | Fusion | FGFR2-WAC | PR |
| 22 | Cholangiocarcinoma | Fusion | FGFR3-TACC3 | SD |
| 23 | Cholangiocarcinoma | Fusion | FGFR2-TACC2 | PR |
| 24 | Cholangiocarcinoma | Fusion | FGFR2-KIAA1598 | PR |
| 25 | Cholangiocarcinoma | Fusion | FGFR2-CD2AP | SD |
| 26 | Cholangiocarcinoma | Fusion | FGFR2-TACC2 | PR |
| 27 | Cholangiocarcinoma | Fusion | FGFR2-AMOT | SD |
| 28 | Cholangiocarcinoma | Fusion | FGFR2-BICC1 | SD |
| 29 | Cholangiocarcinoma | Mutation | FGFR2-C382R | SD |
| 30 | Cholangiocarcinoma | Fusion | FGFR2-CFAP57 | PD |
| 31 | Cholangiocarcinoma | Fusion | FGFR3-TACC3 | SD |
| 32 | High-grade Glioma | Fusion | FGFR3-TACC3 | PR |
| 33 | High-grade Glioma | Fusion | FGFR3-TACC3 | SD |
| 34 | High-grade Glioma | Fusion | FGFR3-ENOX1 | PD |
| 35 | High-grade Glioma | Fusion | FGFR3-TACC3 | PD |
| 36 | High-grade Glioma | Fusion | FGFR3-TACC3 | SD |
| 37 | High-grade Glioma | Fusion | FGFR3-TACC3 | PD |
| 38 | High-grade Glioma | Fusion | FGFR3-TACC3 | PR |
| 39 | High-grade Glioma | Fusion | FGFR3-TACC3 | PR |
| 40 | High-grade Glioma | Fusion | FGFR3-TACC3 | NE |
| 41 | High-grade Glioma | Fusion | FGFR3-TACC3 | PD |
| 42 | High-grade Glioma | Fusion | FGFR3-TACC3 | SD |
| 43 | High-grade Glioma | Fusion | FGFR1-TACC1 | SD |
| 44 | High-grade Glioma | Fusion | FGFR3-TACC3 | NE |
| 45 | High-grade Glioma | Fusion | FGFR3-TACC3 | SD |
| 46 | High-grade Glioma | Fusion | FGFR3-MYH14 | PD |
| 47 | High-grade Glioma | Fusion | FGFR3-TACC3 | SD |
| 48 | High-grade Glioma | Fusion | FGFR3-TMEM247 | PD |
| 49 | High-grade Glioma | Fusion | FGFR3-TACC3 | NE |
| 50 | High-grade Glioma | Fusion | FGFR3-TACC3 | NE |
| 51 | High-grade Glioma | Fusion | FGFR3-TACC3 | SD |
| 52 | High-grade Glioma | Fusion | FGFR3-TACC3 | SD |
| 53 | High-grade Glioma | Fusion | FGFR3-TACC3 | SD |
| 54 | High-grade Glioma | Fusion | FGFR3-TACC3 | NE |
| 55 | High-grade Glioma | Fusion | FGFR3-TACC3 | PD |
| 56 | High-grade Glioma | Fusion | FGFR3-TACC3 | PD |
| 57 | High-grade Glioma | Fusion | FGFR3-TACC3 | NE |
| 58 | Pancreatic Cancer | Fusion | FGFR1-MTUS1 | PR |
| 59 | Pancreatic Cancer | Fusion | FGFR2-ATAD2 | PR |
| 60 | Pancreatic Cancer | Fusion | FGFR2-NRBF2 | SD |
| 61 | Pancreatic Cancer | Fusion | FGFR2-ALDH1L1 | NE |
| 62 | Pancreatic Cancer | Fusion | FGFR2-KIF6 | SD |
| 63 | Pancreatic Cancer | Fusion | FGFR2-GPHN | PR |
| 64 | Pancreatic Cancer | Fusion | FGFR2-GKAP1 | PR |
| 65 | Pancreatic Cancer | Fusion | FGFR1-MTUS1 | SD |
| 66 | Pancreatic Cancer | Fusion | FGFR2-CIT | SD |
| 67 | Pancreatic Cancer | Fusion | FGFR2-KCTD1 | SD |
| 68 | Squamous NSCLC | Mutation | FGFR3-S249C | PD |
| 69 | Squamous NSCLC | Fusion | FGFR3-TACC3 | SD |
| 70 | Squamous NSCLC | Fusion | FGFR3-TACC3 | SD |
| 71 | Squamous NSCLC | Mutation | FGFR3-S249C | PR |
| 72 | Squamous NSCLC | Fusion | FGFR3-TACC3 | PR |
| 73 | Squamous NSCLC | Fusion | FGFR3-TACC3 | SD |
| 74 | Squamous NSCLC | Mutation | FGFR3-S249C | PD |
| 75 | Colorectal Cancer | Mutation | FGFR2-L770V | SD |
| 76 | Colorectal Cancer | Mutation | FGFR3-A500T | SD |
| 77 | Colorectal Cancer | Mutation | FGFR3-F384L | PD |
| 78 | Colorectal Cancer | Fusion | FGFR3-TACC3 | PD |
| 79 | Colorectal Cancer | Fusion | FGFR3-TACC3 | SD |
| 80 | Ovarian Cancer | Fusion | FGFR1-RHPN2 | NE |
| 81 | Ovarian Cancer | Mutation | FGFR3-S249C | PR |
| 82 | Ovarian Cancer | Fusion | FGFR2-AGAP1 | PD |
| 83 | Ovarian Cancer | Fusion | FGFR2-CLOCK | NE |
| 84 | Endometrial Cancer | Mutation | FGFR2-C382R | PR |
| 85 | Endometrial Cancer | Mutation | FGFR2-L551F | SD |
| 86 | Endometrial Cancer | Mutation | FGFR2-C382R | PR |
| 87 | Endometrial Cancer | Mutation | FGFR2-Y375C | SD |
| 88 | Endometrial Cancer | Mutation | FGFR2-D101Y | NE |
| 89 | Low-grade Glioma | Mutation | FGFR1-K656E | PD |
| 90 | Low-grade Glioma | Mutation | FGFR1-K656E | CR |
| 91 | Low-grade Glioma | Fusion | FGFR3-TACC3 | NE |
| 92 | Low-grade Glioma | Fusion | FGFR1-TACC1 | SD |
| 93 | Gastric Cancer | Mutation | FGFR2-Y375C | SD |
| 94 | Gastric Cancer | Mutation | FGFR3-S249C | SD |
| 95 | Gastric Cancer | Mutation | FGFR3-A500T | SD |
| 96 | Gastric Cancer | Fusion | FGFR3-TACC3 | SD |
| 97 | Cervical Cancer | Mutation | FGFR3-S249C | SD |
| 98 | Cervical Cancer | Mutation | FGFR3-S249C | SD |
| 99 | Cervical Cancer | Mutation | FGFR3-S249C | SD |
| 100 | Breast Cancer | Fusion | FGFR1-WHSC1L1 | PD |
| 101 | Breast Cancer | Mutation | FGFR2-K659M | SD |
| 102 | Breast Cancer | Fusion | FGFR1-TACC1 | SD |
| 103 | Breast Cancer | Fusion | FGFR2-TCERG1L | PD |
| 104 | Breast Cancer | Fusion | FGFR2-FKBP15 | SD |
| 105 | Breast Cancer | Mutation | FGFR2-C382R | uPR |
| 106 | Breast Cancer | Fusion | FGFR2-TBC1D4 | PR |
| 107 | Breast Cancer | Fusion | FGFR2-TACC2 | NE |
| 108 | Esophageal Cancer | Mutation | FGFR3-R248C | PR |
| 109 | Esophageal Cancer | Fusion | FGFR3-TACC3 | SD |
| 110 | Squamous Cell Head | Fusion | FGFR3-TACC3 | SD |
| | and Neck Cancers | | | |
| 111 | Squamous Cell Head | Fusion | FGFR3-TACC3 | SD |
| | and Neck Cancers | | | |
| 112 | Squamous Cell Head | Fusion | FGFR3-TACC3 | SD |
| | and Neck Cancers | | | |
| 113 | Squamous Cell Head | Mutation | FGFR3-S249C | uPR |
| | and Neck Cancers | | | |
| 114 | Squamous Cell Head | Mutation | FGFR3-S371G | PD |
| | and Neck Cancers | | | |
| 115 | Non-squamous NSCLC | Fusion | FGFR2-BICC1 | PD |
| 116 | Non-squamous NSCLC | Fusion | FGFR3-TACC3 | PR |
| 117 | Non-squamous NSCLC | Fusion | FGFR2-CCDC102A | uPR |
| 118 | Non-squamous NSCLC | Mutation | FGFR2-Y375C | NE |
| 119 | Basocelular carcinoma | Mutation | FGFR2-S252L | SD |
| 120 | Thymic cancer | Fusion | IGSF3-FGFR1 | SD |
| 121 | Carcinoma of unknown | Fusion | FGFR2-TBC1D5 | SD |
| | primary origin | | | |
| 122 | Carcinoma of unknown | Fusion | FGFR2-BICC1 | PR |
| | primary origin | | | |
| 123 | Carcinoma of unknown | Mutation | FGFR3-S249C | PD |
| | primary origin | | | |
| 124 | Prostate cancer | Mutation | FGFR3-R248C | PD |
| 125 | Salivary gland cancer | Mutation | FGFR1-PLAG1; FGFR2- | SD |
| | | and Fusion | C382R | |
| 126 | Salivary gland cancer | Mutation | FGFR2-F276C | PR |
| 127 | Prostate cancer | Fusion | FGFR3-WHSC1 | PD |
| 128 | Carcinoma of unknown | Mutation | FGFR2-Y375C | NE |
| | primary origin | | | |
| 129 | GIST | Mutation | FGFR3-S249F | SD |
| 130 | Parathyroid carcinoma | Fusion | FGFR1-BAG4 | SD |
| 131 | Carcinoma of unknown | Mutation | FGFR2-S267P | SD |
| | primary origin | | | |
| 132 | Soft tissue sarcoma | Mutation | FGFR1-K656E | NE |
| 133 | Cup-syndrome | Fusion | FGFR2-BICC1 | SD |
| 134 | Salivary gland cancer | Mutation | FGFR2-Y375C | NE |
| 135 | Anal adenocarcinoma | Mutation | FGFR3-R248C | NE |

GIST = Gastrointestinal Stromal Tumor.
Partial response (PR);
complete response (CR);
stable disease (SD);
progressive disease (PD);
not evaluable (NE);
unconfirmed partial response (uPR)

The full gene name and UniProt accession number for the fusion partners in Table 9 is provided in Table 10.

TABLE 10

| | | UniProt |
|---|---|---|
| Gene | | accession |
| Abbreviation | Full Gene Name | number |
| AHCYL1 | Adenosylhomocysteinase Like 1 | O43865 |
| AMOT | Angiomotin | Q4VCS5 |
| BICC1 | BicC Family RNA Binding Protein 1 | Q9H694 |
| CD2AP | CD2 Associated Protein | Q9Y5K6 |
| CFAP57 | Cilia And Flagella Associated Protein 57 | Q96MR6 |
| ENOX1 | Ecto-NOX Disulfide-Thiol Exchanger 1 | Q8TC92 |
| KIAA1598 | Shootin 1 | A0MZ66 |
| LGSN | Lengsin, Lens Protein With Glutamine Synthetase | Q5TDP6 |
| | Domain | |
| NOL4 | Nucleolar Protein 4 | O94818 |
| PAWR | Pro-Apoptotic WT1 Regulator | Q96IZ0 |
| POC1B | POC1 Centriolar Protein B | Q8TC44 |
| SYNPO2 | Synaptopodin 2 | Q9UMS6 |
| TACC2 | Transforming Acidic Coiled-Coil Containing Protein 2 | O95359 |
| TBC1D4 | TBC1 Domain Family Member 4 | O60343 |
| TRA2B | Transformer 2 Beta Homolog | P62995 |
| TACC3 | Transforming Acidic Coiled-Coil Containing Protein 3 | Q9Y6A5 |
| MTUS1 | Microtubule Associated Scaffold Protein 1 | Q9ULD2 |
| ATAD2 | ATPase Family AAA Domain Containing 2 | Q6PL18 |
| CIT | Citron Rho-Interacting Serine/Threonine Kinase | O14578 |
| GKAP1 | G Kinase Anchoring Protein 1 | Q5VSY0 |
| GPHN | Gephyrin | Q9NQX3 |
| KIF6 | Kinesin Family Member 6 | Q6ZMV9 |
| NRBF2 | Nuclear Receptor Binding Factor 2 | Q96F24 |
| PTEN | Phosphatase And Tensin Homolog | P60484 |
| TACC1 | Transforming Acidic Coiled-Coil Containing Protein 1 | O75410 |
| ENOX1 | Ecto-NOX Disulfide-Thiol Exchanger 1 | Q8TC92 |
| MYH14 | Myosin Heavy Chain 14 | Q7Z406 |
| TMEM247 | Transmembrane Protein 247 | A6NEH6 |
| RHPN2 | Rhophilin Rho GTPase Binding Protein 2 | Q8IUC4 |
| AGAP1 | ArfGAP With GTPase Domain, Ankyrin Repeat And | Q9UPQ3 |
| | PH Domain 1 | |
| CLOCK | Clock Circadian Regulator | O15516 |
| IGSF3 | Immunoglobulin Superfamily Member 3 | O75054 |
| WHSC1L1 | Nuclear Receptor Binding SET Domain Protein 3 | Q9BZ95 |
| WHSC1 | Nuclear Receptor Binding SET Domain Protein 2 | O96028 |
| TCERG1L | Transcription Elongation Regulator 1 Like | Q5VWI1 |
| WAC | WW Domain Containing Adaptor With Coiled-Coil | Q9BTA9 |
| BAG4 | BAG Cochaperone 4 | O95429 |
| CCDC102A | Coiled-Coil Domain Containing 102A | Q96A19 |
| FKBP15 | FKBP Prolyl Isomerase Family Member 15 | Q5TIM5 |
| KCTD1 | Potassium Channel Tetramerization Domain Containing 1 | Q719H9 |
| PDE3A | Phosphodiesterase 3A | Q14432 |
| TBC1D5 | TBC1 Domain Family Member 5 | Q92609 |
| PLAG1 | Pleiomorphic Adenoma Gene 1 Protein | Q6DJT9 |
| ALDH1L1 | Aldehyde Dehydrogenase 1 Family Member L1 | O75891 |

The following results include data from an efficacy analysis set and a safety analysis set. The efficacy analysis set includes all subjects treated in the broad panel cohort who initiated treatment on or before a set date and have at least two disease evaluations (n=124). All subjects in the efficacy analysis set were treated and followed until the clinical cut-off date in accordance with the protocol, in order to provide adequate follow-up of at least 5 months of objective evidence of clinical activity on all subjects. The safety analysis set, summarized in the Safety Summary section, includes all subjects in the broad panel cohort who were treated until a set date (n=144).

The efficacy analysis set includes subjects with 21 different tumor types, including 2 pediatric patients (12 and 13 years old). Enrolled tumor types include CNS, gynecological, thoracic, and gastrointestinal malignancies as well as rare tumors such as thymic, parathyroid and salivary gland cancer (Table 11). In addition, enrolled tumor types harbor a diverse set of target mutations (28.2%) or fusions (72.6%) affecting FGFR1 (8.9%), FGFR2 (47.6%) or FGFR3 (44.4%) (Table 11).

TABLE 11
Summary of Tumor Histologies by FGFR Gene and Alteration Type; Efficacy

| | Treated | | FGFR Gene | | | | | | FGFR Alteration Type | | | |
|---|---|---|---|---|---|---|---|---|---|---|---|---|
| | Subjects (%) | | FGFR1 | | FGFR2 | | FGFR3 | | Fusion | | Mutation | |
| Total | 124 | (100.0%) | 11 | (8.9%) | 59 | (47.6%) | 55 | (44.4%) | 90 | (72.6%) | 35 | (28.2%) |
| By Tumor Histology | | | | | | | | | | | | |
| Cholangiocarcinoma (CCA) | 31 | (25.0%) | 0 | | 28 | (90.3%) | 3 | (9.7%) | 28 | (90.3%) | 3 | (9.7%) |
| High-grade Glioma (HGG) | 24 | (19.4%) | 1 | (4.2%) | 0 | | 23 | (95.8%) | 24 | (100.0%) | 0 | |
| Pancreatic Cancer (PANCR) | 9 | (7.3%) | 2 | (22.2%) | 7 | (77.8%) | 0 | | 9 | (100.0%) | 0 | |
| Breast Cancer (BRST) | 7 | (5.6%) | 2 | (28.6%) | 5 | (71.4%) | 0 | | 5 | (71.4%) | 2 | (28.6%) |
| Squamous NSCLC (sqNSCLC) | 7 | (5.6%) | 0 | | 0 | | 7 | (100.0%) | 4 | (57.1%) | 3 | (42.9%) |
| Colorectal Cancer (CRC) | 5 | (4.0%) | 0 | | 1 | (20.0%) | 4 | (80.0%) | 2 | (40.0%) | 3 | (60.0%) |
| Endometrial Cancer (EDMTL) | 4 | (3.2%) | 0 | | 4 | (100.0%) | 0 | | 0 | | 4 | (100.0%) |
| Gastric Cancer (GSTRC) | 4 | (3.2%) | 0 | | 1 | (25.0%) | 3 | (75.0%) | 1 | (25.0%) | 3 | (75.0%) |
| Ovarian Cancer (OVAR) | 4 | (3.2%) | 1 | (25.0%) | 2 | (50.0%) | 1 | (25.0%) | 3 | (75.0%) | 1 | (25.0%) |
| Squamous Cell Head and Neck | 4 | (3.2%) | 0 | | 0 | | 4 | (100.0%) | 2 | (50.0%) | 2 | (50.0%) |
| Cancers (HNSCC) | | | | | | | | | | | | |
| Cervical Cancer (CRVX) | 3 | (2.4%) | 0 | | 0 | | 3 | (100.0%) | 0 | | 3 | (100.0%) |
| Low-grade Glioma (LGG) | 3 | (2.4%) | 2 | (66.7%) | 1 | (33.3%) | 0 | | 1 | (33.3%) | 2 | (66.7%) |
| Non-squamous NSCLC | 3 | (2.4%) | 0 | | 2 | (66.7%) | 1 | (33.3%) | 3 | (100.0%) | 0 | |
| (nonsqNSCLC) | | | | | | | | | | | | |
| Esophageal Cancer (ESOPH) | 2 | (1.6%) | 0 | | 0 | | 2 | (100.0%) | 1 | (50.0%) | 1 | (50.0%) |
| Other | 14 | (11.3%) | 3 | (21.4%) | 8 | (57.1%) | 4 | (28.6%) | 7 | (50.0%) | 8 | (57.1%) |
| Carcinoma of Unknown Primary | 6 | (4.8%) | 0 | | 5 | (83.3%) | 1 | (16.7%) | 3 | (50.0%) | 3 | (50.0%) |
| (CUP) | | | | | | | | | | | | |
| Prostate Cancer (PCA) | 2 | (1.6%) | 0 | | 0 | | 2 | (100.0%) | 1 | (50.0%) | 1 | (50.0%) |
| Salivary Gland Cancer (SALIV) | 2 | (1.6%) | 1 | (50.0%) | 2 | (100.0%) | 0 | | 1 | (50.0%) | 2 | (100.0%) |
| Basocellular Carcinoma (BCC) | 1 | (0.8%) | 0 | | 1 | (100.0%) | 0 | | 0 | | 1 | (100.0%) |
| Gastrointestinal Stromal Tumor | 1 | (0.8%) | 0 | | 0 | | 1 | (100.0%) | 0 | | 1 | (100.0%) |
| (GIST) | | | | | | | | | | | | |

| Parathyroid Carcinoma (PTHCA) | 1 | (0.8%) | 1 | (100.0%) | 0 | 0 | 1 | (100.0%) | 0 |
|---|---|---|---|---|---|---|---|---|---|
| Thymic Cancer (THYM) | 1 | (0.8%) | 1 | (100.0%) | 0 | | 1 | (100.0%) | 0 |

Treated subjects are a heavily pre-treated patient population with significant tumor burden. Among subjects with metastatic disease, 84% had visceral metastasis. All 124 treated subjects (100%) received a minimum of one systemic therapy in the advanced setting as required per protocol. The median number of lines of prior systematic therapy was 2 (Range 1-9). Specifically, 35/124 (28.2%) subjects received 2 prior lines of systemic therapies and 55/124 (44.4%) subjects received 3 or more prior treatments. In addition, 83/124 (66.9%) subjects received prior cancer-related surgery and 52.4% (65/124) received prior radiotherapy. For example, among subjects with HGG, which include subjects with GBM, 20/24 (83.3%) received prior surgery, 24/24 (100%) received prior radiotherapy, 24/24 (100%) received at least one prior line of systemic therapy and 18/24 (75%) received 2 or more lines of systemic therapy. Among subjects with CCA, (31/31) 100^{%} received at least one prior line of systemic therapy, including 30/31 (96.8%) who received prior platinum-based chemotherapy and 19/31 (61.3%) who received 2 or more lines of therapy. Similarly, subjects with pancreatic cancer for which there are no further standard therapeutic options in the metastatic setting to which they were eligible, with a median of 3 prior lines of anti-cancer therapies (Range 1-9). These therapies include established regimens in first and subsequent lines such as FOLFIRINOX, gemcitabine monotherapy/doublets and irinotecan-containing doublets, as appropriate for the individual subject. Subjects with breast cancer, where a wide range of systemic therapies can be used depending on molecular and other criteria, were also heavily pretreated with a median of 5 prior lines of therapy prior to enrollment (Range 2-9). Notably, across the entire efficacy analysis set, only 9/124 (7.3%) subjects responded to the last line of prior therapy per investigator assessment. All treated subjects were confirmed by the investigator to have fulfilled protocol inclusion criteria for which there are no further standard of care options with known established clinical benefit for the underlying tumor type, or the subject's inability to tolerate those therapies. Table 12 provides a summary of disease characteristics for the efficacy analysis set.

TABLE 12
Summary of Disease Characteristics
at Baseline; Efficacy Analysis Set

| | Total |
|---|---|
| Analysis Set: Treated Subjects | 124 |
| Baseline ECOG | |
| N | 122 |
| 0 | 37 (30.3%) |
| 1 | 85 (69.7%) |
| Visceral metastasis | |
| N | 100 |
| Yes | 84 (84.0%) |
| Prior Radiotherapy | |
| N | 124 |
| Yes | 65 (52.4%) |
| Prior Cancer-Related Surgery/Procedure | |
| N | 124 |
| Yes | 83 (66.9%) |
| Prior Systemic Therapy | |
| N | 124 |
| Chemotherapy | 122 (98.4%) |
| Immunotherapy | 26 (21.0%) |
| Other systemic therapy | 55 (44.4%) |
| Number of prior lines of anti-cancer therapies | |
| N | 124 |
| 1 | 34 (27.4%) |
| 2 | 35 (28.2%) |
| ≥3 | 55 (44.4%) |
| Median | 2.00 |
| Range | (1.0; 9.0) |

Visceral metastasis is only analyzed for subjects with confirmed metastatic disease (N = 100); ECOG is only reported for subjects ≥18 years of age (N = 122); 2 adolescent subjects with a Lansky Score of ≥80 are included in the analysis.

### Summary of Response and Durability

The median follow-up for the 124 subjects in the efficacy analysis set is 11.07 months (95% CI; 9.76, 11.27). Thirty-six out of 124 treated subjects had a confirmed response per investigator assessment, leading to an overall response rate (ORR: CR+PR) of 29.0% (95% CI; 21.2%, 37.9%), including 3 subjects with a complete response (CR) and 33 subjects with a partial response (PR). All responses were confirmed with subsequent disease evaluations in accordance with RECIST 1.1 or RANO criteria. Objective response rates to erdafitinib were observed across 12 different tumor types as demonstrated in Table 13.

TABLE 13
Summary of Efficacy Across Tumor Histologies; Efficacy Analysis Set

| | N (Treated | Objective Response Rate | |
|---|---|---|---|
| Tumor Type | Subjects) | n(%) (95% CI) | Median DOR (95% CI) |
| Total | 124 | 36 (29.0%) (21.2%, 37.9%) | 6.93 (4.60, 9.63) |
| CCA | 31 | 13 (41.9%) (24.5%, 60.9%) | 6.93 (4.24, 9.63) |
| HGG | 24 | 5 (20.8%) (7.1%, 42.2%) | NE (4.24, NE) |
| PANCR | 9 | 4 (44.4%) (13.7%, 78.8%) | 8.48 (4.14, NE) |
| BRST | 7 | 3 (42.9%) (9.9%, 81.6%) | 6.08 (NE, NE) |
| sqNSCLC | 7 | 2 (28.6%) (3.7%, 71%) | 4.85 (2.76, NE) |
| CRC | 5 | 0 (NE, NE) | |
| EDMTL | 4 | 2 (50.0%) (6.8%, 93.2%) | NE (4.17, NE) |
| GSTRC | 4 | 0 (NE, NE) | |
| OVAR | 4 | 1 (25.0%) (0.6%, 80.6%) | 7.10 (NE, NE) |
| HNSCC | 4 | 0 (NE, NE) | |
| CRVX | 3 | 0 (NE, NE) | |
| LGG | 3 | 1 (33.3%) (0.8%, 90.6%) | NE (NE, NE) |
| nonsqNSCLC | 3 | 1 (33.3%) (0.8%, 90.6%) | NE (NE, NE) |
| ESOPH | 2 | 1 (50.0%) (1.3%, 98.7%) | 2.79 (NE, NE) |
| Other | 14 | 3 (21.4%) (4.7%, 50.8%) | NE (4.01, NE) |
| CUP | 6 | 2 (33.3%) (4.3%, 77.7%) | 4.01 (NE, NE) |
| PCA | 2 | 0 (NE, NE) | |
| SALIV | 2 | 1 (50.0%) (1.3%, 98.7%) | NE (NE, NE) |
| BCC | 1 | 0 (NE, NE) | |
| GIST | 1 | 0 (NE, NE) | |
| PTHCA | 1 | 0 (NE, NE) | |
| THYM | 1 | 0 (NE, NE) | |

Note:
Objective response is defined as the percentage of subjects achieving CR or PR; Disease control rate is defined as the percentage of subjects achieving CR, PR, or SD; Clinical benefit rate is defined as the percentage of subjects achieving CR, PR, or durable disease (duration of at least 4 months).

Responses were observed across target FGFR mutations (1 CR and 8 PRs) and fusions (2 CRs and 25 PRs), with a comparable ORR of 25.7% (95% CI; 12.5%, 43.3%) in target mutations (9/35), and 30.0% (95% CI; 20.8%, 40.6%) in fusions (27/90). Responses were observed in tumors bearing fusions or target mutations in FGFR1 (1 CR, and 2 PRs out of 11), FGFR2 (2 CRs, and 22 PRs out of 59) and FGFR3 genes (9 PRs out of 55). Responses were not dominated by one mutation or fusion, highlighting a diverse response profile across not only alterations types (mutations/fusions) and FGFR genes (FGFR1-3), but across multiple variants within those categories. Additional information on FGFR alterations identified in each treated subject, including details on underlying tumor type and response are provided in Table 14. Furthermore, responses were observed in subjects who were exposed to a varying number of prior lines of systemic therapy (including 1, 2, or multiple lines of therapy), and all of whom have no other treatment options with established clinical benefit.

TABLE 14
Listing of FGFR alteration, Best Response, Duration of Response and PFS for Each Subject

| | | | Best | | Total | |
|---|---|---|---|---|---|---|
| | | | Overall | | Treatment | |
| | Subject | | Response | DOR | Duration | PFS |
| Tumor Histology | Age | FGFR Alteration | (INV) (months) | (months) | (months) | (months) |
| CCA | 61 | FGFR2-TBC1D4 Fusion | PR | 2.8 | 4.3 | 5.5 |
| | 53 | FGFR2-LGSN Fusion | SD | | 2.2 | 2.5 |
| | 60 | FGFR2-TRA2B Fusion | PR | 9.6 | 11.1 | 11.0 |
| | 28 | FGFR2-BICC1 Fusion | PR | 5.5 | 8.3 | 6.9 |
| | 53 | FGFR2-BICC1 Fusion | PR | 9.7 | 11.0 | 10.8 |
| | 24 | FGFR2-BICC1 Fusion | SD | | 10.4 | 10.3 |
| | 69 | FGFR2-BICC1 Fusion | SD | | 6.9 | 6.6 |
| | 59 | FGFR2-NOL4 Fusion | SD | | 5.0 | 5.3 |
| | 63 | FGFR2-C382R Mutation | SD | | 8.3 | 6.8 |
| | 60 | FGFR2-BICC1 Fusion | PR | 4.2 | 7.6 | 5.6 |
| | 30 | FGFR2-PAWR Fusion | CR | 6.9 | 15.6 | 8.2 |
| | 55 | FGFR2-PDE3A Fusion | PD | | 3.1 | 1.3 |
| | 71 | FGFR2-AHCYL1 Fusion | SD | | 3.7 | 4.4 |
| | 40 | FGFR2-SYNPO2 Fusion | PR | 4.2 | 11.2 | 11.1 |
| | 71 | FGFR2-V395D Mutation | PD | | 1.1 | 1.4 |
| | 53 | FGFR2-BICC1 Fusion | SD | | 11.9 | 11.1+ |
| | 71 | FGFR2-PAWR Fusion | PR | 9.3 | 13.6 | 10.7 |
| | 47 | FGFR2-ENOX1 Fusion | PR | 4.6 | 10.7 | 5.7 |
| | 56 | FGFR3-TACC3 Fusion | SD | | 11.1 | 11.1 |
| | 52 | FGFR2-POC1B Fusion | SD | | 4.6 | 4.2 |
| | 40 | FGFR2-WAC Fusion | PR | 6.7+ | 10.6 | 12.4+ |
| | 61 | FGFR3-TACC3 Fusion | SD | | 9.0 | 9.4 |
| | 48 | FGFR2-TACC2 Fusion | PR | 5.0 | 7.6 | 7.6 |
| | 31 | FGFR2-KIAA1598 Fusion | CR | 9.7+ | 10.3 | 11.1+ |
| | 56 | FGFR2-CD2AP Fusion | SD | | 1.6 | 1.9 |
| | 77 | FGFR2-TACC2 Fusion | PR | 6.9+ | 6.3 | 8.1+ |
| | 59 | FGFR2-AMOT Fusion | SD | | 13.1 | 12.5+ |
| | 38 | FGFR2-BICC1 Fusion | SD | | 10.6 | 10.4 |
| | 59 | FGFR2-C382R Mutation | SD | | 11.8 | 10.9+ |
| | 52 | FGFR2-CFAP57 Fusion | PD | | 2.7 | 1.3 |
| | 67 | FGFR3-TACC3 Fusion | SD | | 2.7 | 2.7 |
| HGG | 39 | FGFR3-TACC3 Fusion | PR | 4.2 | 8.0 | 8.4 |
| | 64 | FGFR3-TACC3 Fusion | SD | | 3.8 | 5.2 |
| | 53 | FGFR3-ENOX1 Fusion | PD | | 1.3 | 1.1 |
| | 70 | FGFR3-TACC3 Fusion | PD | | 0.8 | 0.7 |
| | 54 | FGFR3-TACC3 Fusion | SD | | 5.5 | 5.4 |
| | 40 | FGFR3-TACC3 Fusion | PD | | 1.6 | 1.8 |
| | 55 | FGFR3-TACC3 Fusion | PR | 6.5+ | 13.1 | 10.9+ |
| | 63 | FGFR3-TACC3 Fusion | PR | 6.9+ | 12.4 | 11.3+ |
| | 38 | FGFR3-TACC3 Fusion | PD | | 1.9 | 1.1 |
| | 53 | FGFR3-TACC3 Fusion | SD | | 11.2 | 11.1+ |
| | 13 | FGFR1-TACC1 Fusion | PR | 1.9+ | 10.6 | 9.8+ |
| | 45 | FGFR3-TACC3 Fusion | PR | 1.6+ | 5.0 | 4.1+ |
| | 58 | FGFR3-TACC3 Fusion | SD | | 4.6 | 5.3 |
| | 69 | FGFR3-MYH14 Fusion | PD | | 0.7 | 0.5 |
| | 53 | FGFR3-TACC3 Fusion | SD | | 2.1 | 2.6 |
| | 56 | FGFR3-TMEM247 Fusion | PD | | 1.3 | 0.7 |
| | 61 | FGFR3-TACC3 Fusion | NE | | 0.1 | 0.0+ |
| | 64 | FGFR3-TACC3 Fusion | SD | | 6.0 | 5.8+ |
| | 45 | FGFR3-TACC3 Fusion | SD | | 5.9 | 4.1+ |
| | 56 | FGFR3-TACC3 Fusion | SD | | 1.2 | 1.7 |
| | 59 | FGFR3-TACC3 Fusion | SD | | 3.4 | 2.6 |
| | 38 | FGFR3-TACC3 Fusion | SD | | 3.3 | 3.3 |
| | 49 | FGFR3-TACC3 Fusion | PD | | 1.0 | 1.2 |
| | 68 | FGFR3-TACC3 Fusion | PD | | 2.1 | 1.4 |
| PANCR | 34 | FGFR1-MTUS1 Fusion | PR | 4.1 | 5.5 | 5.4 |
| | 53 | FGFR2-ATAD2 Fusion | PR | 10.1+ | 14.9 | 14.3+ |
| | 60 | FGFR2-NRBF2 Fusion | SD | | 9.6 | 8.2 |
| | 78 | FGFR2-ALDHIL1 Fusion | NE | | 0.5 | 0.0+ |
| | 75 | FGFR2-KIF6 Fusion | SD | | 5.8 | 5.6 |
| | 56 | FGFR2-GPHN Fusion | PR | 8.5 | 10.5 | 9.6 |
| | 44 | FGFR2-GKAP1 Fusion | PR | 5.6+ | 9.9 | 8.5+ |
| | 73 | FGFR1-MTUS1 Fusion | SD | | 4.8 | 5.3 |
| | 69 | FGFR2-CIT Fusion | SD | | 3.4 | 3.8 |
| BRST | 55 | FGFR1-WHSCIL1 Fusion | PD | | 2.8 | 1.2 |
| | 57 | FGFR2-K659M Mutation | SD | | 3.3 | 4.9 |
| | 37 | FGFR1-TACCI Fusion | SD | | 3.5 | 1.5 |
| | 48 | FGFR2-TCERGIL Fusion | PD | | 1.1 | 0.9 |
| | 53 | FGFR2-FKBP15 Fusion | PR | 1.7+ | 5.2 | 4.3+ |
| | 55 | FGFR2-C382R Mutation | PR | 3.3+ | 8.2 | 6.7+ |
| | 53 | FGFR2-TBC1D4 Fusion | PR | 6.1 | 7.4 | 7.3 |
| sqNSCLC | 61 | FGFR3-S249C Mutation | PD | | 1.8 | 1.4 |
| | 57 | FGFR3-TACC3 Fusion | SD | | 2.7 | 3.5 |
| | 77 | FGFR3-TACC3 Fusion | SD | | 1.1 | 1.5 |
| | 54 | FGFR3-S249C Mutation | PR | 6.9 | 7.4 | 8.2 |
| | 57 | FGFR3-TACC3 Fusion | PR | 2.8 | 5.0 | 4.1 |
| | 63 | FGFR3-TACC3 Fusion | SD | | 3.0 | 4.2 |
| | 50 | FGFR3-S249C Mutation | PD | | 1.6 | 1.4 |
| CRC | 60 | FGFR2-L770V Mutation | SD | | 2.8 | 2.6 |
| | 55 | FGFR3-A500T Mutation | SD | | 5.6 | 6.0 |
| | 60 | FGFR3-F384L Mutation | PD | | 0.4 | 0.4 |
| | 56 | FGFR3-TACC3 Fusion | PD | | 1.0 | 0.9 |
| | 70 | FGFR3-TACC3 Fusion | SD | | 7.8 | 6.7+ |
| EDMTL | 62 | FGFR2-C382R Mutation | PR | 10.9+ | 13.3 | 12.3+ |
| | 72 | FGFR2-L551F Mutation | SD | | 1.1 | 1.4 |
| | 78 | FGFR2-C382R Mutation | PR | 4.2 | 5.1 | 5.5 |
| | 47 | FGFR2-Y375C Mutation | SD | | 6.7 | 5.4+ |
| GSTRC | 59 | FGFR2-Y375C Mutation | SD | | 2.5 | 2.6 |
| | 48 | FGFR3-S249C Mutation | SD | | 3.2 | 3.8 |
| | 72 | FGFR3-A500T Mutation | SD | | 2.1 | 2.3 |
| | 68 | FGFR3-TACC3 Fusion | SD | | 2.8 | 2.9 |
| OVAR | 59 | FGFR1-RHPN2 Fusion | NE | | 0.7 | 0.0+ |
| | 51 | FGFR3-S249C Mutation | PR | 7.1 | 11.8 | 8.2 |
| | 73 | FGFR2-AGAP1 Fusion | PD | | 1.4 | 1.3 |
| | 62 | FGFR2-CLOCK Fusion | NE | | 0.7 | 0.0+ |
| HNSCC | 76 | FGFR3-TACC3 Fusion | SD | | 9.7 | 7.5 |
| | 27 | FGFR3-TACC3 Fusion | SD | | 4.6 | 4.0 |
| | 64 | FGFR3-S249C Mutation | SD | | 4.3 | 4.0 |
| | 69 | FGFR3-S371G Mutation | PD | | 0.4 | 0.4 |
| CRVX | 59 | FGFR3-S249C Mutation | SD | | 2.3 | 2.8 |
| | 38 | FGFR3-S249C Mutation | SD | | 4.1 | 4.2 |
| | 37 | FGFR3-S249C Mutation | SD | | 3.0 | 2.9 |
| LGG | 21 | FGFR1-K656E Mutation | PD | | 2.8 | 0.9 |
| | 26 | FGFR1-K656E Mutation | CR | 8.7+ | 10.4 | 10.0+ |
| | 12 | FGFR2-VPS35* Fusion | SD | | 4.5 | 2.6+ |
| nonsqNSCLC | 79 | FGFR2-BICC1 Fusion | PD | | 1.0 | 1.4 |
| | 72 | FGFR3-TACC3 Fusion | PR | 5.8+ | 5.5 | 6.9+ |
| | 63 | FGFR2-CCDC102A Fusion | SD | | 5.5 | 4.1+ |
| ESOPH | 59 | FGFR3-R248C Mutation | PR | 2.8 | 6.0 | 5.6 |
| | 23 | FGFR3-TACC3 Fusion | SD | | 1.3 | 1.3 |
| Other-CUP | 60 | FGFR2-TBC1D5 Fusion | SD | | 10.6 | 8.8 |
| | 55 | FGFR2-BICC1 Fusion | PR | 4.0 | 5.9 | 5.4 |
| | 65 | FGFR3-S249C Mutation | PD | | 2.6 | 2.0 |
| | 54 | FGFR2-Y375C Mutation | SD | | 5.9 | 5.5+ |
| | 62 | FGFR2-S267P Mutation | PR | 1.4+ | 5.7 | 4.1+ |
| | 47 | FGFR2-BICC1 Fusion | SD | | 5.0 | 4.2+ |
| Other-PCA | 74 | FGFR3-R248C Mutation | PD | | 1.1 | 1.2 |
| | 79 | FGFR3-WHSC1 Fusion | PD | | 1.3 | 1.3 |
| Other-SALIV | 41 | FGFR2-C382R Mutation/ | SD | | 4.8 | 7.1+ |
| | | FGFR1-PLAG1 Fusion | | | | |
| | 44 | FGFR2-F276C Mutation | PR | 5.5+ | 7.6 | 6.9+ |
| Other-BCC | 62 | FGFR2-S252L Mutation | SD | | 14.7 | 15.0 |
| Other-GIST | 23 | FGFR3-S249F Mutation | SD | | 5.5 | 4.2+ |
| Other-PTHCA | 62 | FGFR1-BAG4 Fusion | SD | | 5.7 | 4.8+ |
| Other-THYM | 44 | IGSF3-FGFR1 Fusion | SD | | 15.2 | 15.0 |

*Vacuolar Protein Sorting-Associated Protein 35 (VPS35) has UniProt accession number Q96QK1.

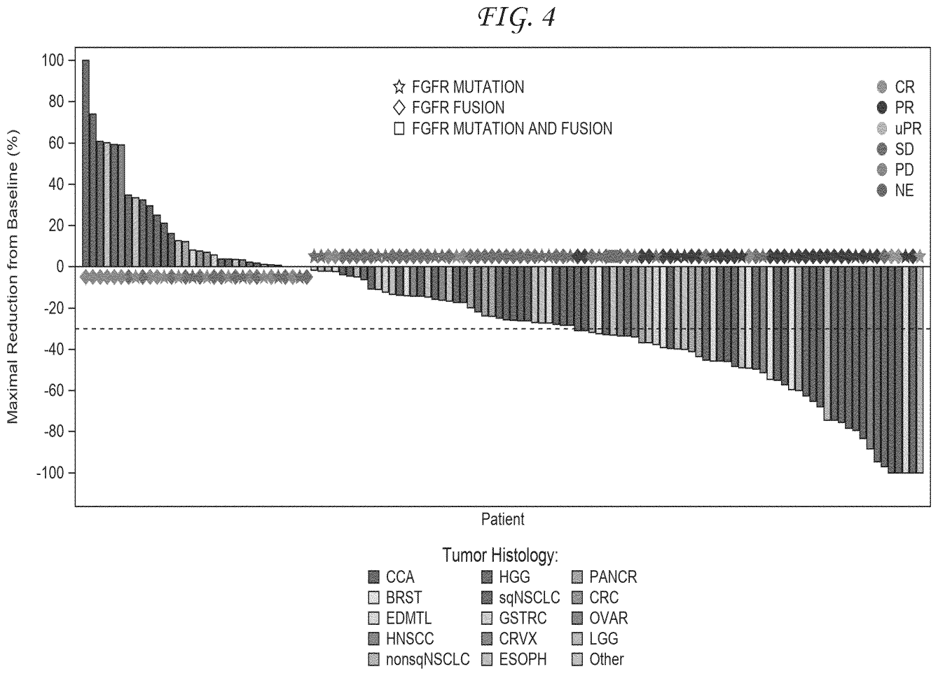

Additionally, 60/124 (48.4%) subjects had stable disease (SD), contributing to a disease control rate (DCR: CR+PR+SD) of 96/124 (77.4%) (95% CI; 69%, 84.4%). Among subjects with SD, the majority demonstrated tumor shrinkage between 10-29% (FIG. 4). Notably, 11/24 (45.8%) subjects with HGG have durable SD or PR with tumor shrinkage and clinical improvement per investigator feedback. In addition, one subject with basal cell carcinoma and an FGFR2 S252L mutation had a substantial clinical improvement of the skin lesion on the face with disease stability for approximately 15.01 months. Another subject with thymic cancer and an IGSF3-FGFR1 fusion also had durable stable disease for approximately 15.05 months. Further, 33 subjects with SD had at least 4 months of disease stability contributing to a clinical benefit rate (CBR: CR+PR+SD>4 months) of 69/124 (55.6%) (95% CI; 46.5%, 64.6%), demonstrating the important clinical benefit among this patient population with significant tumor burden and no standard therapies available.

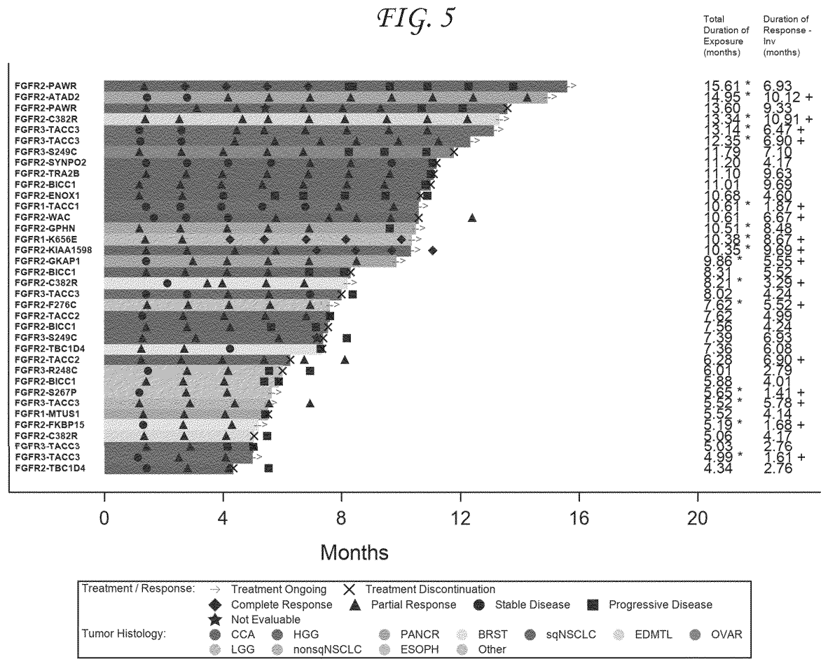

The median time to response was 1.41 months (Range 1.2-7.9). Responses were durable with a median duration of response (DOR) of 6.93 months (95% CI, 4.60, 9.63). The median duration of treatment for responders was 8.26 months. Out of 36 responders (CR/PR), 15 subjects have on-going responses. Furthermore, 16 subjects have a DOR of at least 6 months (7 on-going), 12 responders have a DOR of at least 4 to 6 months (3 on-going), and 8 subjects have a DOR<4 months (5 on-going) (FIG. 5). Notably, durable responses of at least 4 months were observed across tumor types, including CCA, pancreatic, HGG, LGG, breast, endometrial, ovarian cancer, non-squamous NSCLC, and squamous NSCLC. For example, the median DOR for subjects with pancreatic cancer is 8.48 months (95% CI, 4.14, NE). The subject with the longest ongoing PR had endometrial cancer and a DOR of 10.9 months (FIG. 5). The two CCA subjects with CR had a DOR of 6.93 months and 9.69+ months respectively, and the LGG subject with CR had a DOR of 8.67+ months. In addition, 13 subjects were treated for at least 4 weeks beyond radiographic PD, per investigator request, as the subjects continued to show clinical benefit.

### Safety Summary

The safety and tolerability profile observed to date in this study is consistent with the known toxicity profile for erdafitinib. Of the 144 treated BPC subjects included in this analysis, hyperphosphatemia (65.3%), diarrhea (54.2%), stomatitis (49.3%) and dry mouth (47.9%) were the most frequent treatment-emergent adverse events (TEAEs) observed and most were Grade 1-2. Drug-related serious TEAEs occurred in 6.3% of subjects, and 3.5% of subjects had drug-related TEAEs leading to discontinuation. There were no drug related TEAEs leading to death.

### Interim Analysis 3 (IA3) Results

### Overview

The patient population from the IA3 represents approximately 74% (178/˜240) of total study planned patients at the primary analysis. Efficacy as assessed by the IRC demonstrates an ORR of 29.2% (CI 95%; 22.7%, 36.5%) and DOR of 6.90 months (CI 95%; 4.37, 7.95) in a heavily pretreated patient population who have exhausted standard therapies. The efficacy was observed across a wide spectrum of FGFR1-3 mutations and fusions and in multiple tumor types and histologies with confirmed responses in 15 distinct tumor types. The sample size (n=178) at the IA3 provides confidence in the point estimates for ORR and DOR which are clinically meaningful even at the lower bound of the confidence intervals at 22.7% and 4.37 months, respectively. The similar ORR and DOR observed in the IA3 population per IRC assessment (29.2%; 6.90 months) and per investigator assessment (26.4%; 7.10 months), respectively, adds to the confidence in the robustness of the efficacy observed. Clinical benefit is further supported by a clinically meaningful DCR of 72.5% and CBR of 46.1% per TRC, with safety data consistent with the known safety profile of erdafitinib and with an overall favorable risk-benefit ratio.

### Study Population and Disease Characteristics

The analysis set of 178 patients (176 adult and 2 pediatric patients, 12 and 13 years of age) represents 32 distinct tumor types highlighting the wide and diverse range of tumor types and patient populations with target FGFR mutations or fusions. Enrollment was not dominated by any single tumor type and includes patients with CNS, gynecological, thoracic, and gastrointestinal malignancies as well as rare tumors such as thymic, parathyroid and salivary gland cancer. In addition, enrolled tumor types contain a diverse set of target mutations (31.5%) or fusions (68.5%) affecting FGFR1 (9.0%), FGFR2 (48.9%) or FGFR3 (42.1%), including several novel FGFR fusions. No patients with FGFR4 alterations were enrolled for the reported analysis set due to the rarity of these alterations, particularly in adult cancers. Table 15 provides an overview of tumor types, FGFR genes and alteration types enrolled in the BPC.

TABLE 15
Summary of Tumor Histologies by FGFR Gene and Alteration Type

| | Treated | | Altered FGFR Gene | | | | | | FGFR Alteration Type | | | |
|---|---|---|---|---|---|---|---|---|---|---|---|---|
| | Subjects (%) | | FGFR1 | | FGFR2 | | FGFR3 | | Fusion | | Mutation | |
| Total | 178 | (100.0%) | 16 | (9.0%) | 87 | (48.9%) | 75 | (42.1%) | 122 | (68.5%) | 56 | (31.5%) |
| By Tumor Histology [a] | | | | | | | | | | | | |
| Cholangiocarcinoma (CCA) | 31 | (17.4%) | 0 | | 28 | (90.3%) | 3 | (9.7%) | 28 | (90.3%) | 3 | (9.7%) |
| High-grade Glioma (HGG) | 29 | (16.3%) | 1 | (3.4%) | 1 | (3.4%) | 27 | (93.1%) | 29 | (100.0%) | 0 | |
| Breast Cancer (BRST) | 14 | (7.9%) | 2 | (14.3%) | 11 | (78.6%) | 1 | (7.1%) | 10 | (71.4%) | 4 | (28.6%) |
| Pancreatic Cancer (PANCR) | 13 | (7.3%) | 2 | (15.4%) | 11 | (84.6%) | 0 | | 13 | (100.0%) | 0 | |
| Squamous NSCLC (sqNSCLC) | 11 | (6.2%) | 0 | | 2 | (18.2%) | 8 | (72.7%) | 5 | (45.5%) | 5 | (45.5%) |
| Non-squamous NSCLC | 7 | (3.9%) | 0 | | 4 | (57.1%) | 3 | (42.9%) | 4 | (57.1%) | 3 | (42.9%) |
| (nonsqNSCLC) | | | | | | | | | | | | |
| Colorectal Cancer (CRC) | 6 | (3.4%) | 0 | | 2 | (33.3%) | 4 | (66.7%) | 3 | (50.0%) | 3 | (50.0%) |
| Endometrial Cancer (EDMTL) | 6 | (3.4%) | 1 | (16.7%) | 5 | (83.3%) | 0 | | 0 | | 6 | (100.0%) |
| Esophageal Cancer (ESOPH) | 6 | (3.4%) | 0 | | 0 | | 6 | (100.0%) | 4 | (66.7%) | 2 | (33.3%) |
| Low-grade Glioma (LGG) | 6 | (3.4%) | 4 | (66.7%) | 1 | (16.7%) | 1 | (16.7%) | 3 | (50.0%) | 3 | (50.0%) |
| Gastric Cancer (GSTRC) | 5 | (2.8%) | 0 | | 2 | (40.0%) | 3 | (60.0%) | 2 | (40.0%) | 3 | (60.0%) |
| Squamous Cell Head and Neck | 5 | (2.8%) | | | 0 | | | | 3 | (60.0%) | | |
| Cancers (HNSCC) | | | 0 | | | | 5 | (100.0%) | | | 2 | (40.0%) |
| Cervical Cancer (CRVX) | 4 | (2.2%) | 0 | | 0 | | 4 | (100.0%) | 1 | (25.0%) | 3 | (75.0%) |
| Ovarian Cancer (OVAR) | 4 | (2.2%) | 1 | (25.0%) | 2 | (50.0%) | 1 | (25.0%) | 3 | (75.0%) | 1 | (25.0%) |
| Other | 31 | (17.4%) | 5 | (16.1%) | 18 | (58.1%) | 9 | (29.0%) | 14 | (45.2%) | 18 | (58.1%) |
| Carcinoma Of Unknown Primary | 8 | (4.5%) | 0 | | 7 | (87.5%) | 1 | (12.5%) | 5 | (62.5%) | 3 | (37.5%) |
| (CUP) | | | | | | | | | | | | |
| Salivary Gland Cancer (SALIV) | 5 | (2.8%) | 1 | (20.0%) | 5 | (100.0%) | 0 | | 1 | (20.0%) | 5 | (100.0%) |
| Prostate Cancer (PCA) | 2 | (1.1%) | 0 | | 0 | | 2 | (100.0%) | 1 | (50.0%) | 1 | (50.0%) |
| Soft Tissue Sarcoma (STS) | 2 | (1.1%) | 2 | (100.0%) | 0 | | 0 | | 1 | (50.0%) | 1 | (50.0%) |
| Adenoid Cystic Carcinoma (ACC) | 1 | (0.6%) | 0 | | 1 | (100.0%) | 0 | | 0 | | 1 | (100.0%) |
| Anal Adenocarcinoma (ANALCA) | 1 | (0.6%) | 0 | | 0 | | 1 | (100.0%) | 0 | | 1 | (100.0%) |
| Basal Cell Carcinoma (BCC) | 1 | (0.6%) | 0 | | 1 | (100.0%) | 0 | | 0 | | 1 | (100.0%) |
| Conjunctival Epidermoid | 1 | (0.6%) | 0 | | 0 | | 1 | (100.0%) | 0 | | 1 | (100.0%) |
| Carcinoma (CSCC) | | | | | | | | | | | | |
| Duodenal Cancer (DCA) | 1 | (0.6%) | 0 | | 1 | (100.0%) | 0 | | 1 | (100.0%) | 0 | |
| Gallbladder Carcinoma (GBCA) | 1 | (0.6%) | 0 | | 1 | (100.0%) | 0 | | 0 | | 1 | (100.0%) |
| Gastrointestinal Stromal Tumor | 1 | (0.6%) | 0 | | 0 | | 1 | (100.0%) | 0 | | 1 | (100.0%) |
| (GIST) | | | | | | | | | | | | |
| Germ Cell Tumor (GCT) | 1 | (0.6%) | 0 | | 0 | | 1 | (100.0%) | 0 | | 1 | (100.0%) |
| Malignant Small Round Cell Tumor | 1 | (0.6%) | 0 | | 0 | | 1 | (100.0%) | 0 | | 1 | (100.0%) |
| (MSRCT) | | | | | | | | | | | | |

| | | | | | | | | | |
|---|---|---|---|---|---|---|---|---|---|
| Mesothelioma (MESOTH) | 1 | (0.6%) | 0 | 1 | (100.0%) | 0 | 1 | (100.0%) | 0 |

| | | | | | | | | | |
|---|---|---|---|---|---|---|---|---|---|
| Parathyroid Carcinoma (PTHCA) | 1 | (0.6%) | 1 | (100.0%) | 0 | 0 | 1 | (100.0%) | 0 |

| | | | | | | | | | |
|---|---|---|---|---|---|---|---|---|---|
| Testicular Cancer (TESTIC) | 1 | (0.6%) | 0 | 0 | 1 | (100.0%) | 1 | (100.0%) | 0 |

| | | | | | | | | | |
|---|---|---|---|---|---|---|---|---|---|
| Thymic Cancer (THYM) | 1 | (0.6%) | 1 | (100.0%) | 0 | 0 | 1 | (100.0%) | 0 |

| | | | | | | | | | |
|---|---|---|---|---|---|---|---|---|---|
| Thyroid Carcinoma (THYRO) | 1 | (0.6%) | 0 | 1 | (100.0%) | 0 | 1 | (100.0%) | 0 |

### Objective Response and Duration of Response

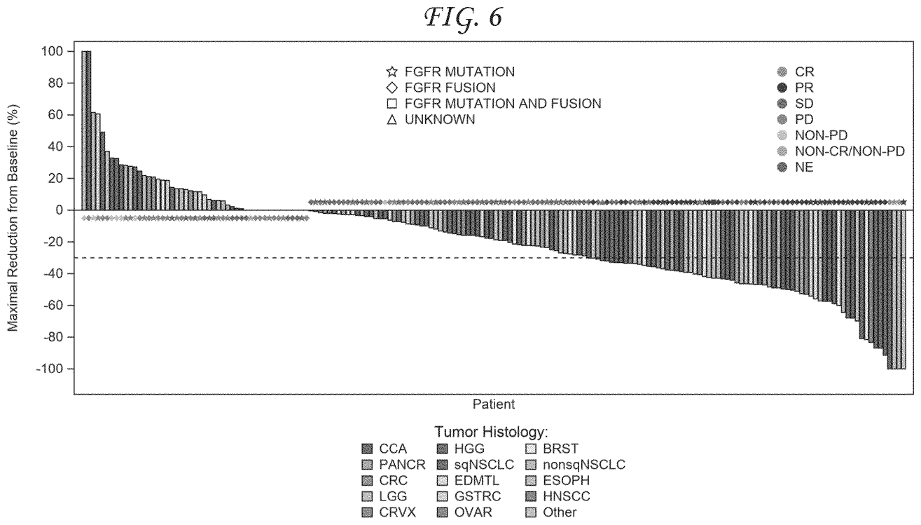

The median efficacy follow-up for the 178 patients in the efficacy analysis set is 12.3 months (95% CI; 9.86, 13.60). Of the 178 treated patients enrolled in the BPC, 52 had a confirmed response per IRC assessment, leading to an overall response rate (ORR: rate of CR+PR) of 29.2% (95% CI; 22.7%, 36.5%), including 3 patients with a CR and 49 patients with a PR. All responses were confirmed with subsequent disease evaluations in accordance with RECIST 1.1 or RANO criteria. Responses were durable, with a median DOR per IRC of 6.90 months (95% CI; 4.37, 7.95). Durable responses to erdafitinib were observed across 15 different tumor types, representing CNS, head and neck, thoracic, gastrointestinal and gynecological malignancies as well as rare tumors such as salivary gland cancer and LGG. Responses to erdafitinib were not dominated by any single tumor type as demonstrated in Table 16 and FIG. 6. Table 17 provides ORR by tumor type for FGFR genes and alteration types. Importantly, the ORR and median DOR per investigator assessment demonstrated similar results to IRC data, with an investigator-assessed ORR of 26.4% (95% CI; 20.1, 33.5) and a median DOR of 7.10 months (95% CI; 5.52, 9.33).

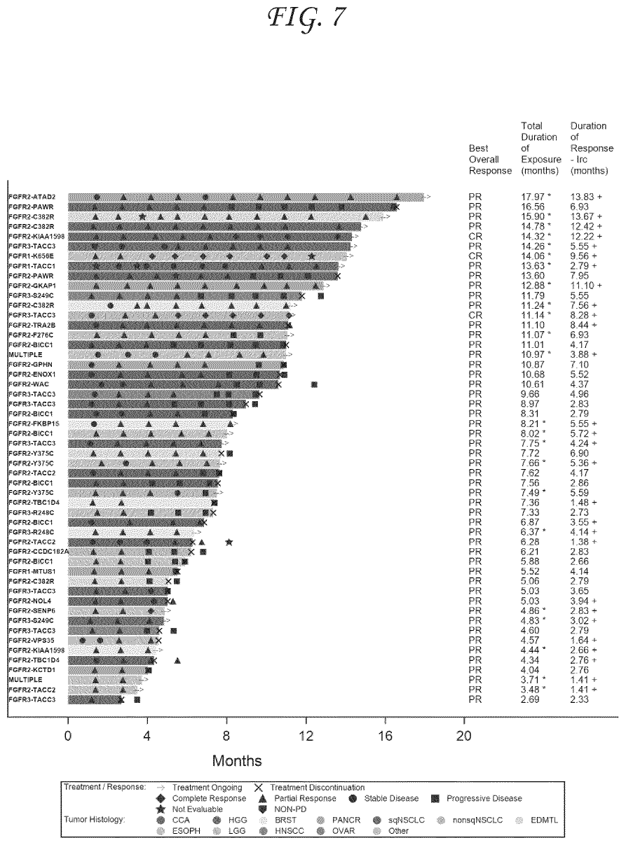

The median duration of treatment for responders was 7.74 months. The median time to response was 1.41 months (range: 1.1 to 9.8). Among 52 responders (CR/PR), 21 patients had ongoing responses at the time of the CCO. Furthermore, 14 patients had a DOR of at least 6 months (8 ongoing), 14 responders had a DOR of at least 4 to 6 months (6 ongoing), and 24 patients had a DOR<4 months (7 ongoing) (FIG. 7). Responses of at least 4 months were observed across a diverse set of tumor types, including CCA, pancreatic, HGG, LGG, breast, endometrial, ovarian, non-squamous NSCLC, squamous cell head and neck, and other carcinomas. Notably, in patients with pancreatic cancer, the median DOR was 7.1 months (95% CI; 2.76, NE). The longest ongoing PRs were in 1 patient with pancreatic cancer (DOR of 13.8+ months) and 1 patient with endometrial cancer (DOR of 13.7+ months) (FIG. 7). Three patients have ongoing confirmed CRs: one patient with CCA has a DOR of 12.2+ months, one patient with LGG has a DOR of 9.6+ months, one patient with non-squamous NSCLC has a DOR of 8.3+ months. In addition, 24 patients were treated for at least 4 weeks beyond the investigator assessment of radiographic progressive disease, per site request, as the patients continued to show clinical benefit.

TABLE 16
Comparison of Key Efficacy Endpoints Between Independent Radiographic Review and Investigator Assessment by Tumor
Histology (Including Other Histology Details) (Broad Panel Cohort); Treated Subjects (Study 42756493CAN2002)

| | | Independent Radiographic Review | | Investigator Assessment | |
|---|---|---|---|---|---|
| | N | Confirmed Objective | | Confirmed Objective Response | |
| | (Treated | Response Rate by IRC | Median DOR by IRC | Rate by INV | Median DOR by INV |
| | Subjects) | n (%) (95% CI) | (95% CI) | n (%) (95% CI) | (95% CI) |
| Total | 178 | 52 (29.2%) (22.7%, 36.5%) | 6.90 (4.37, 7.95) | 47 (26.4%) (20.1%, 33.5%) | 7.10 (5.52, 9.33) |
| CCA | 31 | 16 (51.6%) (33.1%, 69.8%) | 5.52 (2.86, NE) | 13 (41.9%) (24.5%, 60.9%) | 6.93 (4.24, 9.33) |
| HGG | 29 | 3 (10.3%) (2.2%, 27.4%) | NE (NE, NE) | 6 (20.7%) (8.0%, 39.7%) | NE (4.24, NE) |
| BRST | 14 | 5 (35.7%) (12.8%, 64.9%) | NE (NE, NE) | 6 (42.9%) (17.7%, 71.1%) | NE (6.08, NE) |
| PANCR | 13 | 5 (38.5%) (13.9%, 68.4%) | 7.10 (2.76, NE) | 4 (30.8%) (9.1%, 61.4%) | 8.48 (4.14, NE) |
| sqNSCLC | 11 | 3 (27.3%) (6.0%, 61.0%) | 3.65 (2.33, NE) | 3 (27.3%) (6.0%, 61.0%) | 6.93 (2.76, NE) |
| nonsqNSCLC | 7 | 3 (42.9%) (9.9%, 81.6%) | 5.59 (2.83, NE) | 1 (14.3%) (0.4%, 57.9%) | NE (NE, NE) |
| CRC | 6 | 0 (NE, NE) | | 0 (NE, NE) | |
| EDMTL | 6 | 3 (50.0%) (11.8%, 88.2%) | 6.90 (2.79, NE) | 2 (33.3%) (4.3%, 77.7%) | NE (4.17, NE) |
| ESOPH | 6 | 1 (16.7%) (0.4%, 64.1%) | 2.73 (NE, NE) | 1 (16.7%) (0.4%, 64.1%) | 2.79 (NE, NE) |
| LGG | 6 | 2 (33.3%) (4.3%, 77.7%) | NE (NE, NE) | 1 (16.7%) (0.4%, 64.1%) | NE (NE, NE) |
| GSTRC | 5 | 0 (NE, NE) | | 0 (NE, NE) | |
| HNSCC | 5 | 2 (40.0%) (5.3%, 85.3%) | 3.88 (2.79, NE) | 0 (NE, NE) | |
| CRVX | 4 | 0 (NE, NE) | | 0 (NE, NE) | |
| OVAR | 4 | 1 (25.0%) (0.6%, 80.6%) | 5.55 (NE, NE) | 1 (25.0%) (0.6%, 80.6%) | 7.10 (NE, NE) |
| Other | 31 | 8 (25.8%) (11.9%, 44.6%) | 6.93 (2.66, NE) | 9 (29.0%) (14.2%, 48.0%) | 5.55 (4.01, NE) |
| CUP | 8 | 2 (25.0%) (3.2%, 65.1%) | NE (2.66, NE) | 2 (25.0%) (3.2%, 65.1%) | 4.09 (4.01, NE) |
| SALIV | 5 | 4 (80.0%) (28.4%, 99.5%) | 6.93 (NE, NE) | 5 (100.0%) (47.8%, 100.0%) | 6.93 (NE, NE) |
| PCA | 2 | 0 (NE, NE) | | 0 (NE, NE) | |
| STS | 2 | 0 (NE, NE) | | 0 (NE, NE) | |
| Others^{a} | 14 | 2 (14.3%) (1.8%, 42.8%) | NE (NE, NE) | 2 (14.3%) (1.8%, 42.8%) | NE (NE, NE) |

CCA = Cholangiocarcinoma;
HGG = High-grade Glioma;
BRST = Breast Cancer;
PANCR = Pancreatic Cancer;
sqNSCLC = Squamous NSCLC;
nonsqNSCLC = Non-squamous NSCLC;
CRC = Colorectal Cancer;
EDMTL = Endometrial Cancer;
ESOPH = Esophageal Cancer;
LGG = Low-grade Glioma;
GSTRC = Gastric Cancer;
HNSCC = Squamous Cell Head and Neck Cancers;
CRVX = Cervical Cancer;
OVAR-Ovarian Cancer;
CUP = Carcinoma of Unknown Primary;
SALIV = Salivary Gland Cancer;
PCA = Prostate Cancer;
STS = Soft Tissue Sarcoma
^{a}Tumor histologies with 1 subject treated are grouped into others (Tumor types: Adenoid Cystic Carcinoma, Anal Adenocarcinoma, Basal Cell Carcinoma, Conjunctival Epidermoid Carcinoma, Duodenal Cancer, Gallbladder Carcinoma, Gastrointestinal Stromal Tumor, Germ Cell Tumor, Malignant Small Round Cell Tumor, Mesothelioma, Parathyroid Carcinoma, Testicular Cancer, Thymic Cancer, Thyroid Carcinoma). In the “Others” group, response was achieved in 1 subject with duodenal cancer and 1 subject with thyroid carcinoma.

TABLE 17

| | Any FGFR | | | FGFR1 | | | FGFR2 | | | FGFR3 | | |
|---|---|---|---|---|---|---|---|---|---|---|---|---|
| | | ORR | | | ORR | | | ORR | | | ORR | |
| Tumor Type | N | n (%) | | N | n (%) | | N | n (%) | | N | n (%) | |
| Total | 178 | 52 | (29.2) | 16 | 4 | (25.0) | 87 | 37 | (42.5) | 75 | 12 | (16.0)³ |
| CCA | 31 | 16 | (51.6) | 0 | — | | 28 | 15 | (53.6) | 3 | 1 | (33.3) |
| HGG | 29 | 3 | (10.3) | 1 | 1 | (100) | 1 | 0 | | 27 | 2 | (7.4) |
| BRST | 14 | 5 | (35.7) | 2 | 0 | | 11 | 4 | (36.4) | 1 | 1 | (100) |

| | | | | | | | | | | | |
|---|---|---|---|---|---|---|---|---|---|---|---|
| PANCR | 13 | 5 | (38.5) | 2 | 1 | (50.0) | 11 | 4 | (36.4) | — | — |

| sqNSCLC | 11 | 3 | (27.3) | — | — | 2 | 0 | | 8 | 3 | (37.5) |
|---|---|---|---|---|---|---|---|---|---|---|---|
| nonsqNSCLC | 7 | 3 | (42.9) | — | — | 4 | 2 | (50.0) | 3 | 1 | (33.3) |

| | | | | | | | | | | | |
|---|---|---|---|---|---|---|---|---|---|---|---|
| LGG | 6 | 2 | (33.3) | 4 | 1 | (25.0) | 1 | 1 | (100) | 1 | 0 |

| CRC | 6 | 0 | | — | — | 2 | 0 | 4 | 0 | |
|---|---|---|---|---|---|---|---|---|---|---|
| ESOPH | 6 | 1 | (16.7) | — | — | — | — | 6 | 1 | (16.7) |

| | | | | | | | | | | |
|---|---|---|---|---|---|---|---|---|---|---|
| EDMTL | 6 | 3 | (50) | 1 | 0 | 5 | 3 | (60.0) | — | — |

| GSTRC | 5 | 0 | | — | — | 2 | 0 | 3 | 0 | |
|---|---|---|---|---|---|---|---|---|---|---|
| HNSCC | 5 | 2 | (40) | — | — | — | — | 5 | 2 | (40.0) |
| OVAR | 4 | 1 | (25) | 1 | 0 | 2 | 0 | 1 | 1 | (100) |

| CRVX | 4 | 0 | | — | — | | — | — | | 4 | 0 |
|---|---|---|---|---|---|---|---|---|---|---|---|
| Other | 31 | 8 | (25.8) | 5 | 1 | (20.0)¹ | 18 | 8 | (44.4)² | 9 | 0 |

### Subgroup Analysis

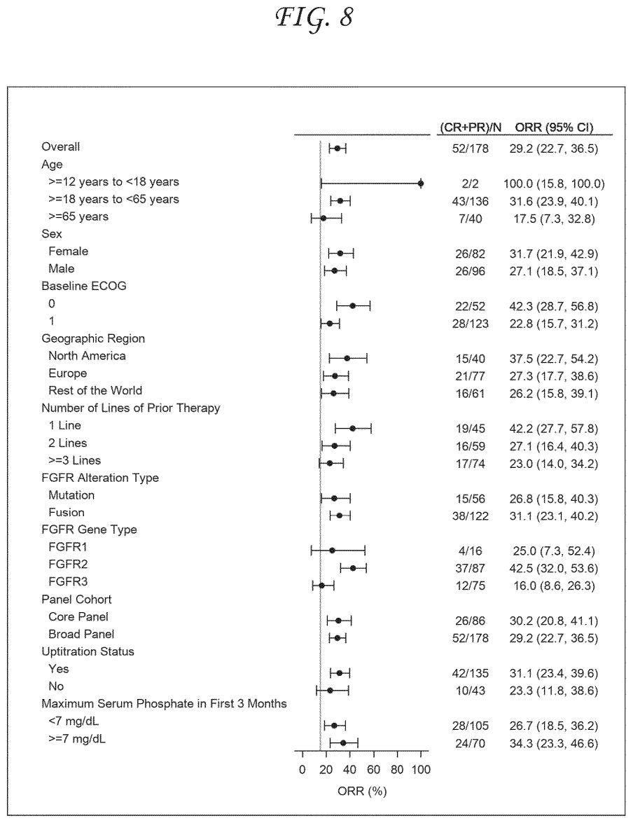

Subgroup analyses of the confirmed ORR, based on IRC, demonstrated that erdafitinib therapy delivered consistent clinical benefit across prespecified subgroups, including age, sex, baseline ECOG status, geographic region, number of lines of prior therapy, FGFR alteration type, and FGFR gene type (FIG. 8), with similar ORR and overlapping 95% CI.

Responses in the analysis set were observed for both BPC and Core Panel cohorts with a comparable ORR of 29.2% in the BPC (95% CI; 22.7%, 36.5%) and 30.2% in the Core Panel Cohort (95% CI; 20.8%, 41.1%). In addition, responses were observed across target FGFR mutations (1 CR and 14 PRs) and fusions (2 CRs and 36 PRs), with a comparable ORR of 26.8% (95% CI; 15.8%, 40.3%) in target mutations (15/56), and 31.3% o (95% CI; 23.1%, 40.2%) in fusions (38/122). Responses were observed in tumors bearing fusions or target mutations in FGFR1 (1 CR, 3 PR of 16), FGFR2 (1 CR, 36 PRs of 87) and FGFR3 genes (1 CR, 11 PRs of 75). Responses were not dominated by any single mutation or fusion, highlighting a diverse response profile across not only alteration types (mutations/fusions) and FGFR genes (FGFR1-3), but across multiple variants within those categories.

### Efficacy Across Tumor Types

As a tumor-agnostic study, the primary objective of Study CAN2002 is to evaluate the efficacy of erdafitinib in terms of ORR by IRC across solid tumors utilizing applicable response evaluation criteria (RECIST 1.1 for non-CNS tumors; RANO criteria for CNS tumors).

Table 18 provides details on secondary endpoints to further characterize clinical activity. Across all tumor types, a DCR of 72.5%, CBR of 46.1%, PFS of 4.2 months and OS of 10.9 months was observed. In addition, 72.5% of patients demonstrated shrinkage of target lesions as shown in FIG. 6. These data support the aforementioned ORR and DOR results of erdafitinib in the tumor agnostic setting. Clinical activity was evaluated for individual tumor types. For example, in HGG, with an ORR by IRC of 10.3% (3/29) (20.7% [6/29] by investigator), 15 of 29 patients (51.7%) had a decrease in sum of product of perpendicular diameters. In addition, 34.5% (10/29) of patients demonstrated clinical benefit, including 7 patients with stable disease of at least 4 months and 3 patients with partial response. Treatment is ongoing for 8 of 29 patients, including 4 patients who have been on treatment for at least 12 months. Among patients with confirmed responses, responses were deep with 2 patients having >75% decrease in sum of product of perpendicular diameters. Responses were clinically meaningful (median: not reached).

In certain tumor types, no confirmed responses have been observed for the reported analysis set. These are tumor types where only a small number of patients was enrolled, reflecting the particularly rare occurrence of FGFR alterations in these tumor entities. These include colorectal cancer (n=6); gastric cancer (n=5) and cervical cancer (n=4), as well as a number of tumor types with 1-2 patients enrolled (Table 18). Despite lack of a confirmed response, several patients with these tumor types demonstrated meaningful clinical benefit on study treatment. For example, among patients with colorectal cancer, gastric cancer, cervical cancer, thymic cancer, parathyroid cancer, GIST, adenoid cystic cancer, basal cell cancer, and soft tissue sarcoma are ones with stable disease of at least 4 months and patients with evidence of tumor shrinkage. These include 2 patients with basal cell cancer and thymic cancer with a PFS of 14.75 months and 15.05 months, respectively. Patients with parathyroid cancer, adenoid cystic cancer and soft tissue sarcoma continue to be on treatment at the timepoint of clinical cut-off date (CCO) without a PFS event.

TABLE 18
Summary of Efficacy by Tumor Histologies (Including Other Histology Details) -
Independent Radiographic Review (Broad Panel Cohort); Treated Subjects

| | N | Confirmed Objective | | Disease Control | Clinical Benefit | | |
|---|---|---|---|---|---|---|---|
| | (Treated | Response Rate n(%) | Median DOR | Rate n(%) | Rate n(%) | Median PFS | Median OS |
| | Subjects) | (95% CI) | (95% CI) | (95% CI) | (95% CI) | (95% CI) | (95% CI) |
| Total | 178 | 52 (29.2%) | 6.90 (4.37, 7.95) | 129 (72.5%) | 82 (46.1%) | 4.21 (4.04, 5.52) | 10.94 (7.89, 14.26) |
| | | (22.7%, 36.5%) | | (65.3%, 78.9%) | (38.6%, 53.7%) | | |
| CCA | 31 | 16 (51.6%) | 5.52 (2.86, NE) | 30 (96.8%) | 24 (77.4%) | 8.25 (6.41, 9.72) | 14.46 (12.09, NE) |
| | | (33.1%, 69.8%) | | (83.3%, 99.9%) | (58.9%, 90.4%) | | |
| HGG | 29 | 3 (10.3%) | NE (NE, NE) | 16 (55.2%) | 10 (34.5%) | 3.86 (2.96, 5.19) | 6.49 (3.75, 14.39) |
| | | (2.2%, 27.4%) | | (35.7%, 73.6%) | (17.9%, 54.3%) | | |
| BRST | 14 | 5 (35.7%) | NE (NE, NE) | 9 (64.3%) | 6 (42.9%) | 4.17 (1.22, NE) | 8.87 (4.86, NE) |
| | | (12.8%, 64.9%) | | (35.1%, 87.2%) | (17.7%, 71.1%) | | |
| PANCR | 13 | 5 (38.5%) | 7.10 (2.76, NE) | 12 (92.3%) | 6 (46.2%) | 4.76 (3.02, NE) | 12.12 (7.26, NE) |
| | | (13.9%, 68.4%) | | (64.0%, 99.8%) | (19.2%, 74.9%) | | |
| sqNSCLC | 11 | 3 (27.3%) | 3.65 (2.33, NE) | 10 (90.9%) | 3 (27.3%) | 5.03 (2.37, NE) | 6.83 (2.37, NE) |
| | | (6.0%, 61.0%) | | (58.7%, 99.8%) | (6.0%, 61.0%) | | |
| nonsqNSCLC | 7 | 3 (42.9%) | 5.59 (2.83, NE) | 4 (57.1%) | 3 (42.9%) | 4.07 (1.38, NE) | NE (2.40, NE) |
| | | (9.9%, 81.6%) | | (18.4%, 90.1%) | (9.9%, 81.6%) | | |
| CRC | 6 | 0 (NE, NE) | | 2 (33.3%) | 1 (16.7%) | 1.15 (0.43, NE) | 5.68 (1.22, NE) |
| | | | | (4.3%, 77.7%) | (0.4%, 64.1%) | | |
| EDMTL | 6 | 3 (50.0%) | 6.90 (2.79, NE) | 4 (66.7%) | 3 (50.0%) | 4.11 (1.31, NE) | NE (1.74, NE) |
| | | (11.8%, 88.2%) | | (22.3%, 95.7%) | (11.8%, 88.2%) | | |
| ESOPH | 6 | 1 (16.7%) | 2.73 (NE, NE) | 2 (33.3%) | 1 (16.7%) | 1.31 (0.30, NE) | 3.42 (1.28, NE) |
| | | (0.4%, 64.1%) | | (4.3%, 77.7%) | (0.4%, 64.1%) | | |
| LGG | 6 | 2 (33.3%) | NE (NE, NE) | 4 (66.7%) | 3 (50.0%) | NE (2.76, NE) | 10.94 (5.72, NE) |
| | | (4.3%, 77.7%) | | (22.3%, 95.7%) | (11.8%, 88.2%) | | |
| GSTRC | 5 | 0 (NE, NE) | | 3 (60.0%) | 0 (NE, NE) | 2.30 (1.22, NE) | 3.29 (2.89, NE) |
| | | | | (14.7%, 94.7%) | | | |
| HNSCC | 5 | 2 (40.0%) | 3.88 (2.79, NE) | 4 (80.0%) | 2 (40.0%) | 4.04 (0.39, NE) | NE (0.39, NE) |
| | | (5.3%, 85.3%) | | (28.4%, 99.5%) | (5.3%, 85.3%) | | |
| CRVX | 4 | 0 (NE, NE) | | 3 (75.0%) | 1 (25.0%) | 3.52 (1.38, NE) | 4.40 (3.48, NE) |
| | | | | (19.4%, 99.4%) | (0.6%, 80.6%) | | |
| OVAR | 4 | 1 (25.0%) | 5.55 (NE, NE) | 2 (50.0%) | 1 (25.0%) | 6.70 (NE, NE) | NE (NE, NE) |
| | | (0.6%, 80.6%) | | (6.8%, 93.2%) | (0.6%, 80.6%) | | |
| Other | 31 | 8 (25.8%) | 6.93 (2.66, NE) | 24 (77.4%) | 18 (58.1%) | 5.52 (2.76, 8.61) | NE (3.94, NE) |
| | | (11.9%, 44.6%) | | (58.9%, 90.4%) | (39.1%, 75.5%) | | |
| CUP | 8 | 2 (25.0%) | NE (2.66, NE) | 7 (87.5%) | 5 (62.5%) | 4.78 (2.04, NE) | 11.99 (2.99, NE) |
| | | (3.2%, 65.1%) | | (47.3%, 99.7%) | (24.5%, 91.5%) | | |
| SALIV | 5 | 4 (80.0%) | 6.93 (NE, NE) | 5 (100.0%) | 5 (100.0%) | NE (8.34, NE) | NE (NE, NE) |
| | | (28.4%, 99.5%) | | (47.8%, 100,0%) | (47.8%, 100.0%) | | |
| PCA | 2 | 0 (NE, NE) | | 0 (NE, NE) | 0 (NE, NE) | 1.26 (1.18, NE) | NE (3.48, NE) |
| STS | 2 | 0 (NE, NE) | | 1 (50.0%) | 1 (50.0%) | NE (3.94, NE) | NE (3.94, NE) |
| | | | | (1.3%, 98.7%) | (1.3%, 98.7%) | | |
| Others^{b} | 14 | 2 (14.3%) | NE (NE, NE) | 11 (78.6%) | 7 (50.0%) | 4.39 (1.41, NE) | NE (3.29, NE) |
| | | (1.8%, 42.8%) | | (49.2%, 95.3%) | (23.0%, 77.0%) | | |

CCA = Cholangiocarcinoma;
HGG = High-grade Glioma;
BRST = Breast Cancer;
PANCR = Pancreatic Cancer;
sqNSCLC = Squamous NSCLC;
nonsqNSCLC = Non-squamous NSCLC;
CRC = Colorectal Cancer;
EDMTL = Endometrial Cancer;
ESOPH = Esophageal Cancer;
LGG = Low-grade Glioma;
GSTRC = Gastric Cancer;
HNSCC = Squamous Cell Head and Neck Cancers;
CRVX = Cervical Cancer;
OVAR = Ovarian Cancer;
CUP = Carcinoma of Unknown Primary;
SALIV = Salivary Gland Cancer;
PCA = Prostate Cancer;
STS = Soft Tissue Sarcoma
CBR is defined as the proportion of subjects who achieve a best response of CR, PR, or durable SD (defined as a duration of at least 4 months) based on RECIST v1.1. or RANO.
DCR is defined as the proportion of subjects who achieve a best response of CR, PR, or SD based on RECIST v1.1. or RANO.
^{a} The objective response rate includes uCR and uPR as responders and a subject will be classified as uCR and uPR if the latest adequate disease evaluation is CR or PR (which has not been confirmed) and a subsequent valid disease evaluation has not been performed yet at the time of the analysis.
^{b}Tumor histologies with 1 subject treated are grouped into others (Tumor types: Adenoid Cystic Carcinoma, Anal Adenocarcinoma, Basal Cell Carcinoma, Conjunctival Epidermoid Carcinoma, Duodenal Cancer, Gallbladder Carcinoma, Gastrointestinal Stromal Tumor, Germ Cell Tumor, Malignant Small Round Cell Tumor, Mesothelioma, Parathyroid Carcinoma, Testicular Cancer, Thymic Cancer, Thyroid Carcinoma). In the “Others” group, response was achieved in 1 subject with duodenal cancer and 1 subject with thyroid carcinoma.
Note:
Time to event endpoints interpretation limited based on single arm study design

A listing of tumor assessments in the Broad Panel Cohort by individual patient and FGFR Alteration type is provided in Table 19.

TABLE 19
Listing of Tumor Assessment - Independent Radiographic Review (Broad
Panel Cohort); Treated Subjects (Study 42756493CAN2002)

| | | | | | | Total | |
|---|---|---|---|---|---|---|---|
| | | | | Best | | Treatment | |
| Tumor | Subject | | | Overall | DOR | Duration | PFS |
| Histology | ID | Age | FGFR Alteration | Response (IRC) | (months) | (months) | (months) |
| CCA | 100166 | 61 | FGFR2-TBC1D4 Fusion | PR | 2.8+ | 4.3 | 5.5+ |
| | 100183 | 53 | FGFR2-LGSN Fusion | SD | | 2.2 | 2.5 |
| | 100293 | 60 | FGFR2-TRA2B Fusion | PR | 8.4+ | 11.1 | 11.2+ |
| | 100458 | 28 | FGFR2-BICC1 Fusion | PR | 2.8 | 8.3 | 6.9 |
| | 100668 | 53 | FGFR2-BICC1 Fusion | PR | 4.2 | 11.0 | 5.3 |
| | 100673 | 24 | FGFR2-BICC1 Fusion | SD | | 10.7 | 10.3+ |
| | 100690 | 69 | FGFR2-BICC1 Fusion | PR | 3.5+ | 6.9 | 6.6+ |
| | 100884 | 59 | FGFR2-NOL4 Fusion | PR | 3.9+ | 5.0 | 5.3+ |
| | 100935 | 63 | FGFR2-C382R Mutation | SD | | 8.3 | 6.8 |
| | 100953 | 60 | FGFR2-BICC1 Fusion | PR | 2.9 | 7.6 | 4.2 |
| | 101009 | 30 | FGFR2-PAWR Fusion | PR | 6.9 | 16.6 | 8.2 |
| | 101011 | 55 | FGFR2-PDE3A Fusion | SD | | 3.1 | 2.8+ |
| | 101060 | 71 | FGFR2-AHCYL1 Fusion | SD | | 3.7 | 4.3 |
| | 101064 | 40 | FGFR2-SYNPO2 Fusion | SD | | 11.2 | 8.3 |
| | 101106 | 71 | FGFR2-V395D Mutation | SD | | 1.1 | 1.4+ |
| | 101191 | 53 | FGFR2-BICC1 Fusion | SD | | 16.8 | 13.8 |
| | 101223 | 71 | FGFR2-PAWR Fusion | PR | 8.0 | 13.6 | 9.3 |
| | 101250 | 47 | FGFR2-ENOX1 Fusion | PR | 5.5 | 10.7 | 8.1 |
| | 101281 | 56 | FGFR3-TACC3 Fusion | SD | | 14.5 | 9.7 |
| | 101285 | 52 | FGFR2-POCIB Fusion | SD | | 4.6 | 6.4 |
| | 101360 | 40 | FGFR2-WAC Fusion | PR | 4.4 | 10.6 | 8.5 |
| | 101446 | 61 | FGFR3-TACC3 Fusion | PR | 2.8 | 9.0 | 5.4 |
| | 101561 | 48 | FGFR2-TACC2 Fusion | PR | 4.2 | 7.6 | 6.8 |
| | 101564 | 31 | FGFR2-KIAA1598 Fusion | CR | 12.2+ | 14.3 | 13.6+ |
| | 101674 | 56 | FGFR2-CD2AP Fusion | SD | | 1.6 | 1.9 |
| | 101757 | 77 | FGFR2-TACC2 Fusion | PR | 1.4+ | 6.3 | 6.7+ |
| | 101783 | 59 | FGFR2-AMOT Fusion | SD | | 16.6 | 15.2 |
| | 102331 | 38 | FGFR2-BICC1 Fusion | SD | | 10.6 | 10.6 |
| | 102384 | 59 | FGFR2-C382R Mutation | PR | 12.4+ | 14.8 | 13.7+ |
| | 102447 | 52 | FGFR2-CFAP57 Fusion | PD | | 2.7 | 1.3 |
| | 102477 | 67 | FGFR3-TACC3 Fusion | SD | | 2.7 | 4.2 |
| HGG | 100664 | 39 | FGFR3-TACC3 Fusion | SD | | 8.0 | 5.6 |
| | 101499 | 64 | FGFR3-TACC3 Fusion | SD | | 3.8 | 5.2 |
| | 101678 | 53 | FGFR3-ENOX1 Fusion | PD | | 1.3 | 1.1 |
| | 101750 | 70 | FGFR3-TACC3 Fusion | PD | | 0.8 | 0.7 |
| | 101769 | 54 | FGFR3-TACC3 Fusion | NON-PD | | 5.5 | 3.2 |
| | 101925 | 40 | FGFR3-TACC3 Fusion | PD | | 1.6 | 1.8 |
| | 101939 | 55 | FGFR3-TACC3 Fusion | SD | | 16.2 | 6.8 |
| | 102109 | 63 | FGFR3-TACC3 Fusion | SD | | 13.8 | 11.3+ |
| | 102123 | 61 | FGFR3-TACC3 Fusion | SD | | 2.6 | 3.9 |
| | 102529 | 38 | FGFR3-TACC3 Fusion | NON-PD | | 1.9 | 3.7 |
| | 102955 | 53 | FGFR3-TACC3 Fusion | PR | 5.6+ | 14.3 | 11.1+ |
| | 103165 | 13 | FGFR1-TACCI Fusion | PR | 2.8+ | 13.6 | 12.5+ |
| | 103295 | 45 | FGFR3-TACC3 Fusion | PR | 4.2+ | 7.8 | 6.7+ |
| | 103481 | 58 | FGFR3-TACC3 Fusion | SD | | 4.6 | 4.2 |
| | 103598 | 69 | FGFR3-MYH14 Fusion | PD | | 0.7 | 0.5 |
| | 104034 | 53 | FGFR3-TACC3 Fusion | SD | | 2.1 | 2.4 |
| | 104441 | 56 | FGFR3-TMEM247 Fusion | PD | | 1.3 | 3.1 |
| | 104667 | 61 | FGFR3-TACC3 Fusion | NE | | 0.1 | 0.0+ |
| | 104686 | 64 | FGFR3-TACC3 Fusion | NON-PD | | 8.7 | 4.0 |
| | 104886 | 45 | FGFR3-TACC3 Fusion | SD | | 8.9 | 4.1+ |
| | 104913 | 56 | FGFR3-TACC3 Fusion | PD | | 1.2 | 1.7 |
| | 105128 | 48 | FGFR3-TACC3 Fusion | SD | | 6.6 | 6.6+ |
| | 105390 | 59 | FGFR3-TACC3 Fusion | NON-PD | | 4.0 | 4.0 |
| | 105497 | 38 | FGFR3-TACC3 Fusion | SD | | 3.2 | 3.3 |
| | 105638 | 49 | FGFR3-TACC3 Fusion | PD | | 1.0 | 1.2 |
| | 105645 | 68 | FGFR3-TACC3 Fusion | NON-PD | | 2.1 | 2.5 |
| | 106635 | 59 | FGFR2-IMPA1 Fusion | SD | | 3.8 | 3.8 |
| | 106848 | 60 | FGFR3-TACC3 Fusion | SD | | 1.5 | 3.0 |
| | 106894 | 51 | FGFR3-TACC3 Fusion | SD | | 5.9 | 5.9 |
| BRST | 103079 | 55 | WHSCIL1-FGFR1 Fusion | PD | | 2.8 | 1.2 |
| | 103266 | 57 | FGFR2-K659M Mutation | PD | | 3.3 | 1.3 |
| | 103400 | 37 | FGFR1-TACC1 Fusion | SD | | 3.5 | 3.5 |
| | 103425 | 48 | FGFR2-TCERGIL Fusion | PD | | 1.1 | 0.9+ |
| | 103760 | 53 | FGFR2-FKBP15 Fusion | PR | 5.6+ | 8.2 | 8.2+ |
| | 104021 | 55 | FGFR2-C382R Mutation | PR | 7.6+ | 11.2 | 11.0+ |
| | 104185 | 53 | FGFR2-TBC1D4 Fusion | PR | 1.5+ | 7.4 | 2.7+ |
| | 106534 | 66 | FGFR2-Y375C Mutation | SD | | 1.8 | 2.8+ |
| | 106559 | 45 | FGFR2-TACC2 Fusion | SD | | 4.1 | 4.2 |
| | 107005 | 74 | FGFR3-R248C Mutation | PR | 4.1+ | 6.4 | 5.5+ |
| | 107159 | 61 | FGFR2-FAM24B Fusion | PD | | 2.8 | 1.2 |
| | 107273 | 41 | FGFR2-BICC1 Fusion | SD | | 6.3 | 5.5+ |
| | 107887 | 62 | CD44-FGFR2 Fusion | PD | | 1.2 | 1.2 |
| | 107909 | 45 | FGFR2-KIAA1598 Fusion | PR | 2.7+ | 4.4 | 4.0+ |
| PANCR | 100038 | 34 | FGFR1-MTUS1 Fusion | PR | 4.1 | 5.5 | 5.4 |
| | 101130 | 70 | FGFR2-KIAA1598 Fusion | SD | | 6.0 | 5.6+ |
| | 101165 | 53 | FGFR2-ATAD2 Fusion | PR | 13.8+ | 18.0 | 16.6+ |
| | 102103 | 60 | FGFR2-NRBF2 Fusion | SD | | 9.6 | 4.1 |
| | 102614 | 78 | FGFR2-ALDHIL1 Fusion | PD | | 0.5 | 0.0+ |
| | 102900 | 75 | FGFR2-KIF6 Fusion | SD | | 5.8 | 3.0 |
| | 103212 | 56 | FGFR2-GPHN Fusion | PR | 7.1 | 10.9 | 9.6 |
| | 103315 | 44 | FGFR2-GKAP1 Fusion | PR | 11.1+ | 12.9 | 12.5+ |
| | 104046 | 73 | FGFR1-MTUS1 Fusion | SD | | 4.9 | 3.3 |
| | 105710 | 69 | FGFR2-CIT Fusion | SD | | 3.4 | 3.8 |
| | 106525 | 61 | FGFR2-KCTD1 Fusion | PR | 2.8 | 4.0 | 4.1 |
| | 108293 | 51 | FGFR2-PAWR Fusion | SD | | 1.2 | 1.4+ |
| | 108436 | 65 | FGFR2-KIAA1598 Fusion | SD | | 2.3 | 1.4+ |
| sqNSCLC | 100268 | 61 | FGFR3-S249C Mutation | SD | | 1.8 | 2.4 |
| | 100554 | 57 | FGFR3-TACC3 Fusion | PR | 2.3 | 2.7 | 3.5 |
| | 100787 | 77 | | SD | | 1.1 | 1.5 |
| | 102818 | 54 | FGFR3-S249C Mutation | NON-CR/NON-PD | | 7.4 | 8.2+ |
| | 104661 | 57 | FGFR3-TACC3 Fusion | PR | 3.6 | 5.0 | 5.0 |
| | 104786 | 63 | FGFR3-TACC3 Fusion | SD | | 3.0 | 2.6 |
| | 105416 | 52 | FGFR2-TACC2 Fusion | SD | | 2.7 | 2.7+ |
| | 107560 | 59 | FGFR3-S249C Mutation | PR | 3.0+ | 4.8 | 4.1+ |
| | 108499 | 64 | FGFR3-R248C Mutation | SD | | 3.6 | 2.7+ |
| | 108610 | 65 | WDR11-FGFR2 Fusion | SD | | 3.4 | 2.7+ |
| | 108672 | 54 | FGFR3-S249C Mutation | SD | | 2.1 | 2.6+ |
| nonsqNSCLC | 103062 | 79 | FGFR2-BICC1 Fusion | PD | | 1.0 | 1.4 |
| | 103974 | 72 | FGFR3-TACC3 Fusion | CR | 8.3+ | 11.1 | 11.1+ |
| | 105655 | 63 | FGFR2-CCDC102A Fusion | PR | 2.8 | 6.2 | 4.1 |
| | 105763 | 50 | FGFR3-S249C Mutation | PD | | 1.6 | 1.4 |
| | 106675 | 63 | FGFR2-Y375C Mutation | PR | 5.6 | 7.5 | 6.9 |
| | 108504 | 69 | FGFR2-TACC2 Fusion | uPR | | 3.6 | 2.6+ |
| | 108842 | 55 | FGFR3-R399C Mutation | PD | | 2.3 | 1.4 |
| CRC | 101188 | 60 | FGFR2-L770V Mutation | PD | | 2.8 | 1.2 |
| | 101827 | 55 | FGFR3-A500T Mutation | SD | | 5.6 | 6.0 |
| | 102182 | 60 | FGFR3-F384L Mutation | PD | | 0.4 | 0.4 |
| | 104629 | 56 | FGFR3-TACC3 Fusion | PD | | 1.0 | 0.9 |
| | 104733 | 70 | FGFR3-TACC3 Fusion | SD | | 8.2 | 5.5 |
| | 107373 | 42 | FGFR2-BICC1 Fusion | PD | | 1.1 | 1.1 |
| EDMTL | 101797 | 62 | FGFR2-C382R Mutation | PR | 13.7+ | 15.9 | 15.0+ |
| | 101919 | 72 | FGFR2-L551F Mutation | PD | | 1.1 | 1.3 |
| | 102432 | 78 | FGFR2-C382R Mutation | PR | 2.8 | 5.1 | 4.1 |
| | 104377 | 47 | FGFR2-Y375C Mutation | PR | 6.9 | 7.7 | 8.1 |
| | 107162 | 55 | FGFR2-D101Y Mutation | SD | | 1.4 | 1.2+ |
| | 107304 | 67 | FGFR1-S125L Mutation | PD | | 1.3 | 1.4 |
| ESOPH | 102759 | 59 | FGFR3-R248C Mutation | PR | 2.7 | 7.3 | 4.2 |
| | 103358 | 63 | FGFR3-TACC3 Fusion | PD | | 1.1 | 1.1 |
| | 104334 | 23 | FGFR3-TACC3 Fusion | PD | | 1.3 | 1.3 |
| | 105848 | 55 | FGFR3-JAKMIP1 Fusion | PD | | 1.2 | 1.3 |
| | 106739 | 68 | FGFR3-TACC3 Fusion | PD | | 2.1 | 0.3 |
| | 106743 | 54 | FGFR3-A500T Mutation | SD | | 2.9 | 2.1 |
| LGG | 101810 | 21 | FGFR1-K656E Mutation | SD | | 2.8 | 2.8 |
| | 102942 | 26 | FGFR1-K656E Mutation | CR | 9.6+ | 14.1 | 10.9+ |
| | 105800 | 12 | FGFR2-VPS35 Fusion | PR | 1.6+ | 4.6 | 4.2+ |
| | 106114 | 32 | FGFR3-TACC3 Fusion | NON-PD | | 1.4 | 1.4+ |
| | 106144 | 22 | FGFR1-TACC1 Fusion | SD | | 7.9 | 5.6+ |
| | 107033 | 21 | FGFR1-K656E Mutation | NON-PD | | 3.4 | 2.8 |
| GSTRC | 100725 | 59 | FGFR2-Y375C Mutation | SD | | 2.5 | 2.6 |
| | 102125 | 48 | FGFR3-S249C Mutation | PD | | 3.2 | 1.5 |
| | 103172 | 72 | FGFR3-A500T Mutation | SD | | 2.1 | 2.3 |
| | 105016 | 68 | FGFR3-TACC3 Fusion | SD | | 5.6 | 4.2 |
| | 106777 | 41 | FGFR2-HTRA1 Fusion | PD | | 2.5 | 1.2 |
| HNSCC | 102201 | 76 | FGFR3-TACC3 Fusion | PR | 5.0 | 9.7 | 7.5 |
| | 104613 | 61 | FGFR3-TACC3 Fusion | SD | | 4.2 | 4.2 |
| | 104701 | 27 | FGFR3-TACC3 Fusion | PR | 2.8 | 4.6 | 4.0 |
| | 104734 | 64 | FGFR3-S249C Mutation | SD | | 4.3 | 4.0 |
| | 105340 | 69 | FGFR3-S371G Mutation | PD | | 0.4 | 0.4 |
| CRVX | 101902 | 59 | FGFR3-S249C Mutation | PD | | 2.3 | 1.4 |
| | 104540 | 38 | FGFR3-S249C Mutation | SD | | 4.1 | 4.2 |
| | 105068 | 37 | FGFR3-S249C Mutation | SD | | 3.0 | 2.9 |
| | 106577 | 45 | FGFR3-TACC3 Fusion | SD | | 4.1 | 4.5 |
| OVAR | 101340 | 59 | RHPN2-FGFR1 Fusion | NE | | 0.7 | 0.0+ |
| | 101660 | 51 | FGFR3-S249C Mutation | PR | 5.6 | 11.8 | 6.7 |
| | 101946 | 73 | FGFR2-AGAP1 Fusion | SD | | 1.4 | 1.3+ |
| | 103031 | 62 | FGFR2-CLOCK Fusion | NE | | 0.7 | 0.0+ |
| Other-CUP | 102067 | 60 | FGFR2-TBC1D5 Fusion | SD | | 13.6 | 8.6 |
| | 103268 | 55 | FGFR2-BICC1 Fusion | PR | 2.7 | 5.9 | 4.0 |
| | 104156 | 65 | FGFR3-S249C Mutation | PD | | 2.6 | 2.0 |
| | 105569 | 54 | FGFR2-Y375C Mutation | SD | | 8.9 | 8.5 |
| | 105735 | 62 | FGFR2-S267P Mutation | SD | | 7.4 | 5.5 |
| | 106061 | 47 | FGFR2-BICC1 Fusion | PR | 5.7+ | 8.0 | 7.1+ |
| | 106871 | 61 | FGFR2-YPEL5 Fusion | SD | | 3.5 | 2.8 |
| | 107597 | 41 | FGFR2-CTNND2 Fusion | SD | | 1.1 | 2.8 |
| Other-SALIV | 104346 | 41 | FGFR2-C382R Mutation / | PR | 3.9+ | 11.0 | 9.9+ |
| | | | FGFR1-PLAG1 Fusion | | | | |
| | 104770 | 44 | FGFR2-F276C Mutation | PR | 6.9 | 11.1 | 8.3 |
| | 106173 | 56 | FGFR2-Y375C Mutation | PR | 5.4+ | 7.7 | 7.0+ |
| | 107414 | 78 | FGFR2-Y375C Mutation | SD | | 5.1 | 4.4+ |
| | 108271 | 53 | FGFR2-E565A Mutation / | PR | 1.4+ | 3.7 | 2.8+ |
| | | | FGFR2-W72C Mutation | | | | |
| Other-PCA | 104188 | 74 | FGFR3-R248C Mutation | PD | | 1.1 | 1.2 |
| | 105096 | 79 | WHSC1-FGFR3 Fusion | PD | | 1.3 | 1.3 |
| Other-STS | 105875 | 39 | FGFR1-K656E Mutation | PD | | 1.3 | 3.9 |
| | 108205 | 74 | FGFR1-MTUS1 Fusion | SD | | 4.3 | 4.2+ |
| Other-ACC | 107961 | 75 | FGFR2-P253L Mutation | SD | | 4.8 | 4.3+ |
| Other-ANALCA | 106957 | 57 | FGFR3-R248C Mutation | PD | | 1.4 | 1.4 |
| Other-BCC | 100252 | 62 | FGFR2-S252L Mutation | SD | | 14.7 | 14.8 |
| Other-CSCC | 108025 | 71 | FGFR3-S249C Mutation | SD | | 1.8 | 1.9 |
| Other-DCA | 108567 | 45 | FGFR2-TACC2 Fusion | PR | 1.4+ | 3.5 | 2.8+ |
| Other-GBCA | 108568 | 76 | FGFR2-Y375C Mutation | PD | | 2.1 | 1.3 |
| Other-GIST | 105589 | 23 | FGFR3-S249F Mutation | SD | | 8.5 | 6.0 |
| Other-GCT | 106589 | 42 | FGFR3-P250R Mutation | PD | | 0.6 | 0.2 |
| Other-MSRCT | 107007 | 57 | FGFR3-S249C Mutation | SD | | 1.9 | 2.8 |
| Other-MESOTH | 108573 | 51 | FGFR2-GOLGA2 Fusion | SD | | 2.7 | 2.7 |
| Other-PTHCA | 105676 | 62 | BAG4-FGFR1 Fusion | SD | | 8.8 | 8.3+ |
| Other-TESTIC | 107847 | 40 | FGFR3-TACC3 Fusion | SD | | 1.7 | 2.1 |
| Other-THYM | 100671 | 44 | IGSF3-FGFR1 Fusion | SD | | 15.2 | 15.0 |
| Other-THYRO | 107122 | 62 | FGFR2-SENP6 Fusion | PR | 2.8+ | 4.9 | 4.2+ |

### Safety of Erdafitinib

The safety analysis presented here is for the same analysis set (n=178) treated in the BPC. The safety and tolerability profile observed to date in Study CAN2002 is consistent with the known toxicity profile of erdafitinib. Review of safety data by the Data Review Committee at Interim Analysis 1, 2 and 3 did not raise new safety concerns or lead to changes in study conduct.

An overall summary of TEAEs is provided in Table 20. All subjects in the BPC (100.0%) experienced at least 1 TEAE. Over half of the patients in the BPC (69.1%) had a TEAE that was Grade 3 or higher and 44.9% had a Grade 3 or higher TEAE that was considered by the investigator to be related to erdafitinib (i.e., drug related). Serious adverse events were reported for 37.6% of patients and were considered drug related for 7.3% of patients. Over half of the patients experienced TEAEs that led to dose interruption (74.7%) or reduction (61.2%), while a smaller percentage of TEAEs led to treatment discontinuation (12.9%). TEAEs leading to death were reported for 13 patients (7.3%), including one death considered related to study treatment per investigator assessment.

The most frequently reported TEAEs for the 178 subjects in the BPC (>30%) were hyperphosphatemia (68.5%), diarrhea (57.9%), stomatitis (52.8%), dry mouth (48.3%), dry skin (33.7%), palmar-plantar erythrodysesthesia syndrome (32.0%), and constipation (30.3%) (Table 21); most TEAEs were Grade 1 or 2 in severity. TEAEs of Grade 3 or higher (≥5%) were stomatitis (9.0%), anaemia (7.9%), alanine aminotransferase increased (5.1%), palmar-plantar erythrodysesthesia syndrome (6.2%), and hyperphosphatemia (5.6%). Serious adverse events of abdominal pain, and pyrexia were reported for 6 patients (3.4% each); those of general physical health deterioration was reported for 5 (2.8%) and diarrhea and pneumonia, were reported for 4 (2.2%) patients; and the other serious adverse events occurred in ≤3 patients each.

TABLE 20
Overall Summary of Treatment-emergent Adverse Events (All
Cohorts); Treated Subjects (Study 42756493CAN2002)

| | Broad Panel Cohort |
|---|---|
| Analysis set: Treated Subjects | 178 |
| Any TEAEs | 178 (100.0%) |
| Drug-related | 171 (96.1%) |
| Grade 3 or higher TEAEs | 123 (69.1%) |
| Drug-related | 80 (44.9%) |
| Serious TEAEs | 67 (37.6%) |
| Drug-related | 13 (7.3%) |
| TEAEs leading to dose reduction | 109 (61.2%) |
| Drug-related | 104 (58.4%) |
| TEAEs leading to dose interruption | 133 (74.7%) |
| Drug-related | 113 (63.5%) |
| TEAEs leading to treatment discontinuation | 23 (12.9%) |
| Drug-related | 13 (7.3%) |
| TEAEs leading to death | 13 (7.3%) |
| Drug-related | 1 (0.6%) |

Key: TEAE = Treatment-emergent adverse event
AEs leading to death are based on AE outcome of Fatal.
Subjects with >1 records are counted only once at corresponding row levels.

TABLE 21
Treatment Emergent Adverse Events (TEAEs) with Frequency
>=10% by Preferred Term (All Cohorts); Treated
Subjects (Study 42756493CAN2002) Broad Panel Cohort

| | Broad Panel Cohort |
|---|---|
| Analysis set: Treated Subjects | 178 |
| Subjects with 1 or more TEAEs | 178 (100.0%) |
| Preferred term | |
| Hyperphosphataemia | 122 (68.5%) |
| Diarrhoea | 103 (57.9%) |
| Stomatitis | 94 (52.8%) |
| Dry mouth | 86 (48.3%) |
| Dry skin | 60 (33.7%) |
| Palmar-plantar erythrodysaesthesia syndrome | 57 (32.0%) |
| Constipation | 54 (30.3%) |
| Alanine aminotransferase increased | 50 (28.1%) |
| Fatigue | 50 (28.1%) |
| Decreased appetite | 45 (25.3%) |
| Aspartate aminotransferase increased | 45 (25.3%) |
| Anaemia | 45 (25.3%) |
| Dry eye | 40 (22.5%) |
| Alopecia | 37 (20.8%) |
| Nausea | 36 (20.2%) |
| Onycholysis | 35 (19.7%) |
| Epistaxis | 31 (17.4%) |
| Nail disorder | 31 (17.4%) |
| Paronychia | 31 (17.4%) |
| Nail discolouration | 30 (16.9%) |
| Arthralgia | 29 (16.3%) |
| Abdominal pain | 28 (15.7%) |
| Vomiting | 27 (15.2%) |
| Dysgeusia | 27 (15.2%) |
| Blood alkaline phosphatase increased | 26 (14.6%) |
| Weight decreased | 24 (13.5%) |
| Vision blurred | 23 (12.9%) |
| Pain in extremity | 21 (11.8%) |
| Asthenia | 19 (10.7%) |
| Urinary tract infection | 19 (10.7%) |
| Back pain | 18 (10.1%) |
| Thrombocytopenia | 18 (10.1%) |

Key: TEAE = treatment-emergent adverse event
Note:
Subjects are counted only once for any given event, regardless of the number of times they actually experienced the event.
Adverse events are coded using MedDRA Version 24.1.

Drug-related TEAEs were reported in 96.1% of subjects in the BPC. The most frequently reported drug-related TEAEs in the BPC (>30%) were hyperphosphatemia (68.5%), stomatitis (52.2%), diarrhea (48.9%), dry mouth (46.6%), dry skin (32.6%), and palmar-plantar erythrodysesthesia syndrome (32.0%). Most drug-related TEAEs were Grade 1, 2, or 3 in severity, with the exception of 1 subject with a Grade 4 TEAEs of cutaneous calcification and calciphylaxis, and 1 subject with a Grade 5 TEAE of pulmonary embolism in the setting of disease progression.

The TEAE of special interest, i.e., central serous retinopathy (CSR), was reported for 26 (14.6%) subjects in the BPC. The most frequently reported preferred terms for CSR for subjects in the BPC were chorioretinopathy (4.5%), detachment of retinal pigment epithelium (3.9%), retinal detachment (3.4%), and subretinal fluid (1.7%); other preferred terms occurred in 1 or 2 subjects. All CSR events were Grade 1 or 2 for subjects in the BPC and none were serious TEAEs. CSR led to dose reduction in 17 (9.6%) subjects in the BPC, and lead to dose interruption in 14 (7.9%) subjects in the BPC; however, no subjects in the BPC discontinued due to CSR.

For patients in the BPC, TEAEs resulted in dose interruptions for 74.7% of patients. The most commonly (≥5%) reported TEAEs leading to dose interruptions were stomatitis (17.4%), palmar-plantar erythrodysesthesia syndrome (14.0%), and diarrhea, paronychia, and hyperphosphatemia (each reported in 6.7% of patients). Treatment-emergent adverse events resulted in discontinuation of treatment for 23 (12.9%) patients, 3 (1.7%) patients discontinued due to a TEAE of palmar-plantar erythrodysesthesia syndrome and general physical health deterioration, while the other TEAEs leading to discontinuation of treatment occurred in ≤2 patients each. Treatment-emergent adverse events resulted in dose reduction for 61.2% of patients; the most common events were stomatitis (13.5%), palmar plantar erythrodysesthesia syndrome (9.0%), onycholysis (8.4%), diarrhoea (7.3%), hyperphosphatemia (5.6%), paronychia (5.1%), chorioretinopathy, fatigue, alanine aminotransferase increased (4.5% each), and dry mouth (3.9%), while the other TEAEs that resulted in dose reduction occurred in <5 patients.

### SUMMARY

The data from the IA3 population with 178 patients demonstrate an impactful ORR by IRC of 29.2% (CI 95% CI; 22.7%, 36.5%) and median DOR of 6.90 months (CI 95% CI; 4.37, 7.95) observed across 15 tumor types with FGFR alterations in patients with advanced/metastatic solid tumors who have exhausted standard of care options. The robust sample size at IA3 provides confidence in the point estimates for ORR and DOR which are clinically meaningful even at the lower bound of the confidence intervals at 22.7% and 4.37 months, respectively. The clinical benefit in the same patient population is further supported by a clinically meaningful DCR of 72.5% and CBR of 46.1%, with safety data consistent with the known safety profile of erdafitinib and with an overall favorable risk-benefit ratio.

### OVERALL CONCLUSION

Findings from molecular screening in the RAGNAR study indicate that clinical trials are feasible in patients with rare genetic alterations by adopting a histology-histology-agnostic design and using both central testing and local testing reports for molecular eligibility screening. This approach helps investigate rare tumors with FGFR alterations, for which histology-specific trials are challenging. In the clinical setting, FGFR alterations were observed in tumor types for which FGFR alterations are not present in genomic databases. Results from the RAGNAR study will help define the benefit of erdafitinib in patients with FGFR-altered advanced solid tumors, for whom there are limited therapeutic options.

No available therapies with established clinical benefit are available for patients with advanced solid tumors with FGFR mutations and fusions that have progressed on or after at least one line of systemic therapy and for whom there are no further available therapies with established clinical benefit or who are unable to tolerate standard therapies. Therefore, supportive care is the only clinically accepted treatment option remaining, leaving these patients with significant unmet need. Further, there are no approved FGFR inhibitors in FGFR-altered solid tumors independent of underlying tumor type. In this heavily pretreated patient population with an exceptionally poor prognosis, the preliminary clinical evidence described herein shows durable responses across different tumor types and across a wide spectrum of FGFR alterations, in particular FGFR 1-3 mutations and fusions, including CCA, pancreatic, HGG, LGG, NSCLC, breast, endometrial and rare cancers in adults and adolescents. Similar frequencies of responses were observed across FGFR mutations and fusions. This preliminary objective evidence of clinical activity indicates a substantial and durable clinical improvement in a FGFR positive, tumor agnostic patient population who have progressed on or after at least one prior line of systemic therapy with no further available effective treatment options. These data support preliminary clinical evidence that erdafitinib may provide substantial improvement over available therapies for a patient population with a high disease burden and a significant unmet medical need.

The examples and embodiments described herein are for illustrative purposes only and various modifications or changes suggested to persons skilled in the art are to be included within the spirit and purview of this application and scope of the appended claims.

1. A method of treating cancer, said method comprising administering a therapeutically effective amount of erdafitinib to a patient who has been diagnosed with cancer and who harbors at least one fibroblast growth factor receptor (FGFR) fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFR1, RHPN2-FGFR1, and RRM2B-FGFR2.

2-4. (canceled)

5. The method of claim 1, wherein (1) the FGFR fusion is FGFR2 CCDC102A and the cancer is non-squamous non-small-cell lung cancer (NSCLC), (2) the FGFR fusion is FGFR2-CCDC147, FGFR2-ENOX1, FGFR2-LCN10, FGFR2-PDE3A, FGFR2-RANBP2, or RRM2B-FGFR2 and the cancer is cholangiocarcinoma, (3) the FGFR fusion is FGFR2-GPHN and the cancer is pancreatic cancer, (4) the FGFR fusion is FGFR3-ENOX1 or FGFR3-TMEM247 and the cancer is a high-grade glioma, (5) the FGFR fusion is IGSF3-FGFR1 and the cancer is a thymic cancer, or (6) the FGFR fusion is RHPN2-FGFR1 and the cancer is ovarian cancer.

6-21. (canceled)

22. A method of treating cancer, said method comprising administering a therapeutically effective amount of erdafitinib to a patient who has been diagnosed with cancer and who harbors at least one fibroblast growth factor receptor (FGFR) genetic alteration, wherein the cancer is cholangiocarcinoma, high-grade glioma, pancreatic cancer, squamous non-small-cell lung cancer (NSCLC), non-squamous NSCLC, breast cancer, colorectal cancer, endometrial cancer, gastric cancer, ovarian cancer, carcinoma of unknown primary origin, cervical cancer, squamous cell head and neck cancer, esophageal cancer, low-grade glioma, prostate cancer, salivary gland cancer, basal cell carcinoma, thymic cancer, small intestine adenocarcinoma, hepatocellular carcinoma, microcystic adnexal carcinoma, spinocellular carcinoma, gastrointestinal stromal tumor, parathyroid carcinoma, soft tissue sarcoma, adenoid cystic carcinoma, anal adenocarcinoma, conjunctival epidermoid carcinoma, duodenal cancer, gallbladder carcinoma, germ cell tumor, malignant small round cell tumor, mesothelioma, testicular cancer, or thyroid carcinoma.

23. (canceled)

24. The method of claim 22, wherein the cancer is cholangiocarcinoma, high-grade glioma, pancreatic cancer, squamous non-small-cell lung cancer (NSCLC), non-squamous NSCLC, breast cancer, endometrial cancer, ovarian cancer, carcinoma of unknown primary origin, squamous cell head and neck cancers, esophageal cancer, low-grade glioma, salivary gland cancer, duodenal cancer, or thyroid carcinoma

25. The method of claim 22, wherein the at least one FGFR genetic alteration is an FGFR mutation or an FGFR fusion.

26. The method of claim 22, wherein the at least one FGFR genetic alteration is FGFR1-PLAG1, FGFR2-C382R, FGFR1-BAG4, IGSF3-FGFR1, FGFR1-K656E, FGFR1-MTUS1, FGFR1-RHPN2, FGFR1-TACC1, FGFR1-WHSC1L1, FGFR2-AGAP1, FGFR2-AHCYL1, FGFR2-ALDH1L1, FGFR2-AMOT, FGFR2-ATAD2, FGFR2-BICC1, FGFR2-CCDC102A, FGFR2-CD2AP, FGFR2-CFAP57, FGFR2-CIT, FGFR2-CLOCK, FGFR2-D101Y, FGFR2-ENOX1, FGFR2-F276C, FGFR2-FKBP15, FGFR2-GKAP1, FGFR2-GPHN, FGFR2-K659M, FGFR2-KCTD1, FGFR2-KIAA1598, FGFR2-KIF6, FGFR2-L551F, FGFR2-L770V, FGFR2-LGSN, FGFR2-NOL4, FGFR2-NRBF2, FGFR2-PAWR, FGFR2-PDE3A, FGFR2-POC1B, FGFR2-S252L, FGFR2-S267P, FGFR2-SYNPO2, FGFR2-TACC2, FGFR2-TBC1D4, FGFR2-TBC1D5, FGFR2-TCERG1L, FGFR2-TRA2B, FGFR2-V395D, FGFR2-VPS35, FGFR2-WAC, FGFR2-Y375C, FGFR3-A500T, FGFR3-ENOX1, FGFR3-F384L, FGFR3-MYH14, FGFR3-R248C, FGFR3-S249C, FGFR3-S249F, FGFR3-S371G, FGFR3-TACC3, FGFR3-TMEM247, FGFR3-WHSC1, BAG4-FGFR1, RHPN2-FGFR1, WHSC1L1-FGFR1, WHSC1-FGFR3, CD44-FGFR2, FGFR2-CTNND2, FGFR2-FAM24B, FGFR2-GOLGA2, FGFR2-HTRA1, FGFR2-IMPA1, FGFR2-SENP6, FGFR2-YPEL5, FGFR3-JAKMIP1, WDR11-FGFR2, FGFR1-S125L, FGFR2-E565A, FGFR2-P253L, FGFR2-W72C, FGFR3-P250R, or FGFR3-R399C.

27-28. (canceled)

29. The method of claim 22, wherein the subject received at least one line of systemic therapy prior to said administration of erdafitinib.

30. The method of claim 22, further comprising evaluating a biological sample from the patient for the presence of the at least one FGFR fusion or the at least one FGFR genetic alteration prior to said administration of erdafitinib.

31. The method of claim 30, wherein the biological sample is blood, lymph fluid, bone marrow, a solid tumor sample, or any combination thereof.

32-33. (canceled)

34. The method of claim 22, wherein erdafitinib is administered orally on a continuous daily dosing schedule.

35. The method of claim 22, wherein the patient is 15 years of age or older at the date of first administration of the FGFR inhibitor.

36. The method of claim 35, wherein erdafitinib is administered at a dose of about 8 mg once daily or wherein erdafitinib is administered at a dose of about 9 mg once daily, in particular wherein erdafitinib is administered at a dose of about 8 mg once daily.

37. The method of claim 22, wherein the patient is between 12 years of age and <15 years of age at the date of first administration of said FGFR inhibitor.

38. The method of claim 37, wherein erdafitinib is administered at a dose of about 5 mg once daily or wherein erdafitinib is administered at a dose of about 6 mg once daily or wherein erdafitinib is administered at a dose of about 8 mg once daily, in particular wherein erdafitinib is administered at a dose of about 5 mg once daily.

39. The method of claim 22, wherein the patient is between 6 years of age and <12 years of age at the date of first administration of said FGFR inhibitor.

40. The method of claim 39, wherein erdafitinib is administered at a dose of about 3 mg once daily or wherein erdafitinib is administered at a dose of about 4 mg once daily or wherein erdafitinib is administered at a dose of about 5 mg once daily, in particular wherein erdafitinib is administered at a dose of about 3 mg once daily.

41. The method of claim 1, wherein erdafitinib is administered in a solid dosage form.

42. The method of claim 41, wherein the solid dosage form is a tablet.

43. A method of treating cancer comprising:
evaluating a biological sample for the presence of at least one fibroblast growth factor receptor (FGFR) fusion selected from FGFR2-CCDC102A, FGFR2-CCDC147, FGFR2-ENOX1, FGFR2-GPHN, FGFR2-LCN10, FGFR2-PDE3A, FGFR2-RANBP2, FGFR3-ENOX1, FGFR3-TMEM247, IGSF3-FGFRT, RHPN2-FGFR1, and RRM2B-FGFR2 from a patient who has been diagnosed with cancer; and
administering a therapeutically effective dose of an FGFR inhibitor to the patient if at least one FGFR fusion is present in the sample.

44. The method of claim 43, wherein the FGFR fusion is selected from FGFR2-CCDC102A, FGFR2-ENOX1, FGFR2-GPHN, FGFR2-PDE3A, FGFR3-ENOX1, FGFR3-TMEM247, IGSF3-FGFR1, and RHPN2-FGFR1.

45-48. (canceled)

49. The method of claim 43, wherein the FGFR inhibitor is erdafitinib.

50-61. (canceled)


SEQUENCE LISTING (5 sequences)

SEQ ID NO: 1 - protein, 822 aa, Homo sapiens [sequence omitted]

SEQ ID NO: 2 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 3 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 4 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 5 - DNA, 24 bp, Artificial Sequence
  Description of Artificial Sequence Synthetic oligonucleotide
tttttttttttttttttttttttt